(19) United States
(12) Patent Application Publication
(10) Pub. No.: US 2019/0338015 A1
(43) Pub. Date: Nov. 7, 2019

(54) CELL DEATH INDUCING CHIMERIC ANTIGEN RECEPTORS

(71) Applicant: CELLECTIS, Paris (FR)

(72) Inventors: Alexandre JUILLERAT, New York, NY (US); Philippe DUCHATEAU, Draveil (FR); Laurent POIROT, Paris (FR); Murielle DERRIEN, Paris (FR); Donna Marie STONE, Brisbane, CA (US); Javier Fernando CHAPARRO RIGGERS, San Mateo, CA (US)

(21) Appl. No.: 16/342,139

(22) PCT Filed: Oct. 19, 2017
(86) PCT No.: PCT/EP2017/076801

§ 371 Date: Apr. 15, 2019

Related U.S. Application Data
(60) Provisional application No. 62/436,749, filed on Dec. 20, 2016.
(60) Provisional application No. 62/410,187, filed on Oct. 19, 2016.

(30) Foreign Application Priority Data
Oct. 27, 2016 (DK) PA 2016 70840
Jan. 20, 2017 (DK) PA 2017 70037

(51) Int. Cl. C07K 14/705; C12N 5/0783; A61K 35/17; C07K 14/725; C07K 16/28

(52) U.S. Cl. CPC C07K 14/70578; C12N 5/0636; A61K 35/17; C07K 14/7051; C07K 14/70517; C07K 16/2803; A61K 38/00; C07K 2319/03; C07K 2319/02; C07K 2319/60; C07K 2319/30; C07K 2317/622; C07K 2317/73; C07K 2319/33

(57) ABSTRACT

The invention relates to cell death inducing chimeric antigen receptors (D-CAR). In particular, the present invention relates to cell death inducing chimeric antigen receptors which comprise at least one death domain in their endodomain, including cell death inducing chimeric antigen receptors comprising within their death domains modifications which attenuate the self-association and/or binding to pro-apoptotic or pro-necrotic adaptor proteins, such as FADD or TRADD. Moreover, the present invention relates to an engineered immune cell expressing at its surface a cell death inducing CAR of the present invention and, optionally, an activating chimeric antigen receptor, wherein the extracellular ligand-binding domains of the cell death inducing CAR and the activating CAR bind to different antigens. The engineered immune cell may furthermore comprise at least one edited (e.g., inactivated) gene selected from TCR genes, immune check point genes, genes involved in drug resistance, and combinations thereof.

### CROSS-REFERENCE TO RELATED APPLICATIONS

This application is a national phase application under 35 U.S.C. § 371 of PCT International Application No. PCT/EP2017/076801, filed Oct. 19, 2017, which claims priority to Danish Application No. PA 2017 70037, filed Jan. 20, 2017, U.S. Provisional Application No. 62/436,749, filed Dec. 20, 2016, Danish Application No. PA 2016 70840, filed Oct. 27, 2016, and U.S. Provisional Application No. 62/410,178, filed Oct. 19, 2016, each of which are hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 177,531 Byte ASCII (Text) file named “37739-US-3-PCT_ST25.TXT,” created on Jul. 10, 2019.

### FIELD OF THE INVENTION

The invention relates to cell death inducing chimeric antigen receptors (D-CAR). In particular, the present invention relates to cell death inducing chimeric antigen receptors which comprise at least one death domain in their endodomain, including cell death inducing chimeric antigen receptors comprising within their death domains modifications which attenuate the self-association and/or binding to pro-apoptotic or pro-necrotic adaptor proteins, such as FADD or TRADD. Moreover, the present invention relates to an engineered immune cell expressing at its surface a cell death inducing CAR of the present invention and, optionally, an activating chimeric antigen receptor, wherein the extracellular ligand-binding domains of the cell death inducing CAR and the activating CAR bind to different antigens. The engineered immune cell may furthermore comprise at least one edited (e.g., inactivated) gene selected from TCR genes, immune check point genes, genes involved in drug resistance, and combinations thereof.

### INTRODUCTION

Adoptive immunotherapy, which involves the transfer of autologous or allogenic antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011, Trends Biotechnol 29(11):550-7). Transfer of viral antigen specific T cells is a well-established procedure used for the treatment of transplant associated viral infections and rare viral-related malignancies. Similarly, isolation, engineering and transfer of tumor specific T cells have been shown to be successful in treating melanoma.

Novel specificities in T cells have been successfully generated through the genetic transfer of transgenic T cell receptors or chimeric antigen receptors (CARs) (Jena, Dotti et al. 2010, Blood 116(7):1035-44). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010, Blood 116(7):1035-44). Current immunotherapies are designed to target single antigens on cancer cells.

However, cancer and healthy cells can express the same antigen, even if it is at different levels. Means to control the cytolytic activity of engineered cells towards healthy cells have thus been proposed. Indeed, having the possibility to combine at least 2 different antigens recognized by an engineered immune cell to spare healthy tissue but not cancer cells presents extremely valuable advantage over actual technology for therapeutic purposes.

With this aim, integration of a “modular NOT gate” within a CAR design may provide a strategy to insure safety and expand the number of surface antigens available for therapeutic purposes. Logic gates are the basic building blocks in electronic circuits that perform logical operations. These have input and output signals in the form of 0's and 1's; ‘0’ signifies the absence of signal while ‘1’ signifies its presence. Similar to the electronic logic gates, cellular components can serve as logic gates. Synthetic biology applies many of the principles of engineering to the field of biology in order to create biological devices which can ultimately be integrated into increasingly complex systems. These principles include standardization of parts, modularity, abstraction, reliability, predictability, and uniformity (Adrianantoandro, Basu et al., 2006, Mol Syst Biol 2: 2006.0028). The application of engineering principles to biology is complicated by the inability to predict the functions of even simple devices and modules within the cellular environment. Some of the confounding factors are gene expression noise, mutation, cell death, undefined and changing extracellular environments, and interactions with the cellular context (Adrianantoandro, Basu et al., 2006, Mol Syst Biol 2: 2006.0028). Thus, while synthetic biology offers much promise in developing systems to address challenges faced in the fields of manufacturing, environment and sustainability, and health and medicine, the realization of this potential is currently limited by the diversity of available parts and effective design frameworks (Wang, Wei, et al. 2013).

Different synthetic biology approaches to control the activity of engineered CAR T cells in the environment of the healthy tissue have already been described, including PD1-based NOT gates (also called iCAR). However, as acknowledged by the authors, Fedorov et al. (Sci Transl Med 5(215)), such strategies are extremely difficult to setup and will have severe limitations for clinical applications.

WO2016/100236 discloses modified immune cells comprising a chimeric antigen receptor comprising a membrane-associated polypeptide-region and a first multimerizing region, and a chimeric caspase-based polypeptide comprising a pro-apoptotic polypeptide region and a second multimerizing region, wherein the first and second multimerizing regions bind to a first multimeric ligand. Because this complex architecture requires the intervention of a compound multimerizing the chimeric antigen receptor and the caspase-based pro-apoptotic polypeptide to trigger apoptosis, there is a great risk of escape which cannot be neglected.

WO2016/097231 discloses inhibitory chimeric antigen receptors comprising a polypeptide sequence involved in inducing an inhibitory transduction signal derived from a tumor-necrosis-factor related apoptosis inducing ligand (TRAIL) receptor.

However, there remains a need for further means to control the cytolytic activity of engineered cells towards healthy cells and improve the safety of the CAR technology.

### SUMMARY

Generally, the present invention applies biology principles such as logic “NOT gate” to immune cell technology to improve the safety. To this end, the present inventors have engineered chimeric antigen receptors that will, upon engagement (only) with their target, induce cell death of the immune cells expressing same at their surface. Such engineered chimeric antigen receptors are called cell death inducing chimeric antigen receptors or D-CARs. The cell death may be mediated through intracellular apoptotic or necrotic pathways.

The present invention thus provides in a general aspect a cell death inducing chimeric antigen receptor which comprises at least one death domain (such as at least two death domains) in its endodomain domain.

The present invention further provides in another general aspect engineered immune cells expressing at their surface a cell death inducing CAR of the present invention and, optionally, an cell activating, target specific receptor, such as a target specific chimeric antigen receptor, which enables the engineered immune cells to trigger the destruction of desired pathological target cells (such as tumor cells), upon recognition of a target antigen (such as a tumor antigen). The inventors of the present invention have found that cell death inducing CAR (D-CAR) positive cells optionally expressing an activating CAR (A-CAR) have unexpected and specific properties that are disclosed herein.

The present invention may be summarized by the following items:
1. A cell death inducing chimeric antigen receptor (CAR) which comprises:
   a) at least one ectodomain which comprises an extracellular ligand-binding domain and a hinge;
   b) at least one transmembrane domain; and
   c) at least one endodomain which comprises at least one death domain.
2. The cell death inducing CAR according to item 1, wherein the death domain is derived from a death receptor.
3. The cell death inducing CAR according to item 1 or 2, wherein the death domain is derived from a death domain of a transmembrane receptor of the tumor necrosis factor (TNF) superfamily.
4. The cell death inducing CAR according to any one of items 1 to 3, wherein the death domain is derived from a death domain of a member of the TNFR superfamily selected from the group consisting of: Fas (CD95), DR4, DR5, TNFR1 and DR3.
5. The cell death inducing CAR according to any one of items 1 to 4, wherein the death domain is derived from the death domain of Fas (CD95).
6. The cell death inducing CAR according to any one of items 1 to 4, wherein the death domain is derived from the death domain of DR4.
7. The cell death inducing CAR according to any one of items 1 to 4, wherein the death domain is derived from the death domain of DR5.
8. The cell death inducing CAR according to any one of items 1 to 4, wherein the death domain is derived from the death domain of TNFR1.
9. The cell death inducing CAR according to any one of items 1 to 4, wherein the death domain is derived from the death domain of DR3.
10. The cell death inducing CAR according to any one of items 1 to 4, wherein the death domain comprises, or consists of, an amino acid sequence having at least 80% sequence identity with any one of amino acid sequences set forth in SEQ ID NO: 6 (corresponding to the death domain of human Fas), SEQ ID NO: 7 (corresponding to the death domain of human DR4), SEQ ID NO: 8 (corresponding to the death domain of human DR5), SEQ ID NO: 9 (corresponding to the death domain of human TNFR1) or SEQ ID NO: 10 (corresponding to the death domain of human DR3).
11. The cell death inducing CAR according to any one of items 1 to 4, wherein the death domain comprises, or consists of, an amino acid sequence having at least 80% sequence identity with SEQ ID NO: 6.
12. The cell death inducing CAR according to any one of items 1 to 4, wherein the death domain comprises, or consists of, an amino acid sequence having at least 80% sequence identity with SEQ ID NO: 7.
13. The cell death inducing CAR according to any one of items 1 to 4, wherein the death domain comprises, or consists of, an amino acid sequence having at least 80% sequence identity with SEQ ID NO: 8.
14. The cell death inducing CAR according to any one of items 1 to 4, wherein the death domain comprises, or consists of, an amino acid sequence having at least 80% sequence identity with SEQ ID NO: 9.
15. The cell death inducing CAR according to any one of items 1 to 4, wherein the death domain comprises, or consists of, an amino acid sequence having at least 80% sequence identity with SEQ ID NO: 10.
16. The cell death inducing CAR according to any one of items 1 to 15, wherein the death domain comprises one or more, such as 1 to 15, mutations in its amino acid sequence compared to the amino acid sequence of the wild type death domain from which it is derived, which mutation(s) attenuate(s) the self-association and/or binding to a pro-apoptotic or pro-necrotic adaptor protein.
17. The cell death inducing CAR according to any one of items 1 to 16, wherein the death domain comprises one or more, such as 1 to 15, amino acid substitutions, e.g., non-conservative substitutions, in its amino acid sequence compared to the amino acid sequence of the wild type death domain from which it is derived, which amino acid substitution(s) attenuate(s) the self-association and/or binding to a pro-apoptotic or pro-necrotic adaptor protein.
18. The cell death inducing CAR according to item 17, wherein the one or more amino acid substitutions are at positions corresponding to positions in the full length amino acid sequence of Fas (SEQ ID NO: 1) selected from the group consisting of V245, R250, K251, V254, E256, K258, I259, D260, E261, K263, E272, W281, Y291, K296 and L298.
19. The cell death inducing CAR according to item 17, wherein the one or more amino acid substitutions are at positions corresponding to positions in the full length amino acid sequence of Fas (SEQ ID NO: 1) selected from the group consisting of R250, V254, E256, D260, E261, K263, Y291 and K296.
20. The cell death inducing CAR according to item 17, wherein the one or more amino acid substitutions are at positions corresponding to positions in the full length amino acid sequence of DR4 (SEQ ID NO: 2) selected from the group consisting of W380, R385, Q286, L289, K391, E293, I294, D295, V296, R298, D406, W415, 1425, D430, and L432.
21. The cell death inducing CAR according to item 17, wherein the one or more amino acid substitutions are at positions corresponding to positions in the full length amino acid sequence of DR4 (SEQ ID NO: 2) selected from the group consisting of W380, Q286, K391, E293, D295, R298, D406 and L432.
22. The cell death inducing CAR according to item 17, wherein the one or more amino acid substitutions are at positions corresponding to positions in the full length amino acid sequence of DR5 (SEQ ID NO: 3) selected from the group consisting of W354, R359, K360, L363, D365, E367, I368, K369, V370, K372, D380, W389, V399, D404 and L406.
23. The cell death inducing CAR according to item 17, wherein the one or more amino acid substitutions are at positions corresponding to positions in the full length amino acid sequence of DR5 (SEQ ID NO: 3) selected from the group consisting of W354, K360, D365, E367, K369, K372, D380 and L406.
24. The cell death inducing CAR according to item 17, wherein the one or more amino acid substitutions are at positions corresponding to positions in the full length amino acid sequence of TNFR1 (SEQ ID NO: 4) selected from the group consisting of W371, R376, R377, L380, D382, E384, I385, D386, R387, E389, E398, W407, L418, R423 and L425.
25. The cell death inducing CAR according to item 17, wherein the one or more amino acid substitutions are at positions corresponding to positions in the full length amino acid sequence of DR3 (SEQ ID NO: 5) selected from the group consisting of W347, R352, T353, L356, E358, E360, I361, E362, E365, D373, W382, L390 and L397.
26. The cell death inducing CAR according to any one of items 1 to 4, wherein the death domain comprises, or consists of, an amino acid sequence having at least 80% sequence identity with SEQ ID NO: 6, the amino acid sequence comprising at least one amino acid substitution at a position which corresponds to a position in SEQ ID NO: 6 selected from the group consisting of V16, R21, K22, V25, E27, K29, I30, D31, E32, K34, E43, W52, Y62, K67 and L69.
27. The cell death inducing CAR according to any one of items 1 to 4, wherein the death domain comprises, or consists of, an amino acid sequence which differs from the amino acid sequence of SEQ ID NO: 6 in that one or more amino acid residues at positions selected from the group consisting of V16, R21, K22, V25, E27, K29, I30, D31, E32, K34, E43, W52, Y62, K67 and L69 are substituted.
28. The cell death inducing CAR according to any one of items 1 to 4, wherein the death domain comprises, or consists of, an amino acid sequence having at least 80% sequence identity with SEQ ID NO: 6, the amino acid sequence comprising at least one amino acid substitution at a position which corresponds to a position in SEQ ID NO: 6 selected from the group consisting of R21, V25, E27, D31, E32, K34, Y62 and K67.
29. The cell death inducing CAR according to item 28, wherein the at least one amino acid substitution is selected from the group consisting of R21A, V25N, E27A, D31A, E32A, K34A, Y62D and K67A.
30. The cell death inducing CAR according to item 28 or 29, wherein the at least one amino acid substitution includes K67A.
31. The cell death inducing CAR according to any one of items 1 to 4, wherein the death domain comprises, or consists of, an amino acid sequence which differs from the amino acid sequence of SEQ ID NO: 6 in that one or more amino acid residues at positions selected from the group consisting of R21, V25, E27, D31, E32, K34, Y62 and K67 are substituted. 32. The cell death inducing CAR according to item 31, wherein the one or more substitutions are selected from the group consisting of R21A, V25N, E27A, D31A, E32A, K34A, Y62D and K67A.
33. The cell death inducing CAR according to item 31 or 32, wherein the amino acid sequence includes the substitution K67A.
34. The cell death inducing CAR according to any one of items 1 to 4, wherein the death domain comprises, or consists of, an amino acid sequence having at least 80% sequence identity with SEQ ID NO: 7, the amino acid sequence comprising at least one amino acid substitution at a position which corresponds to a position in SEQ ID NO: 7 selected from the group consisting of: W16, R21, Q22, L25, K27, E29, I30, D31, V32, R34, D42, W51, I61, D66 and L68.
35. The cell death inducing CAR according to any one of items 1 to 4, wherein the death domain comprises, or consists of, an amino acid sequence which differs from the amino acid sequence of SEQ ID NO: 7 in that one or more amino acid residues at positions selected from the group consisting of W16, R21, Q22, L25, K27, E29, I30, D31, V32, R34, D42, W51, I61, D66 and L68 are substituted.
36. The cell death inducing CAR according to any one of items 1 to 4, wherein the death domain comprises, or consists of, an amino acid sequence having at least 80% sequence identity with SEQ ID NO: 7, the amino acid sequence comprising at least one amino acid substitution at a position which corresponds to a position in SEQ ID NO: 7 selected from the group consisting of: W16, Q22, K27, E29, D31, R34, D42 and L68.
37. The cell death inducing CAR according to item 36, wherein the at least one amino acid substitution is selected from the group consisting of: W16A, Q22A, K27A, E29A, D31A, R34A, D42A and L68A.
38. The cell death inducing CAR according to any one of items 1 to 4, wherein the death domain comprises, or consists of, an amino acid sequence which differs from the amino acid sequence of SEQ ID NO: 7 in that one or more amino acid residues at positions selected from the group consisting of W16, Q22, K27, E29, D31, R34, D42 and L68 are substituted.
39. The cell death inducing CAR according to item 38, wherein the substitutions are selected from the group consisting of W16A, Q22A, K27A, E29A, D31A, R34A, D42A and L68A.
40. The cell death inducing CAR according to any one of items 1 to 4, wherein the death domain comprises, or consists of, an amino acid sequence having at least 80% sequence identity with SEQ ID NO: 8, the amino acid sequence comprising at least one amino acid substitution at a position which corresponds to a position in SEQ ID NO: 8 selected from the group consisting of: W16, R21, K22, L25, D27, E29, I30, K31, V32, K34, D42, W51, V61, D66 and L68.
41. The cell death inducing CAR according to any one of items 1 to 4, wherein the death domain comprises, or consists of, an amino acid sequence which differs from the amino acid sequence of SEQ ID NO: 8 in that one or more amino acid residues at positions selected from the group consisting of W16, R21, K22, L25, D27, E29, I30, K31, V32, K34, D42, W51, V61, D66 and L68 are substituted.
42. The cell death inducing CAR according to any one of items 1 to 4, wherein the death domain comprises, or consists of, an amino acid sequence having at least 80% sequence identity with SEQ ID NO: 8, the amino acid sequence comprising at least one amino acid substitution at a position which corresponds to a position in SEQ ID NO: 8 selected from the group consisting of: W16, K22, D27, E29, K31, K34, D42 and L68.
43. The cell death inducing CAR according to item 42, wherein the at least one amino acid substitution is selected from the group consisting of: W16A, K22A, D27A, E29A, K31A, K34A, D42A and L68A.
44. The cell death inducing CAR according to any one of items 1 to 4, wherein the death domain comprises, or consists of, an amino acid sequence which differs from the amino acid sequence of SEQ ID NO: 8 in that one or more amino acid residues at positions selected from the group consisting of W16, K22, D27, E29, K31, K34, D42 and L68 are substituted.
45. The cell death inducing CAR according to item 44, wherein the one or more substitutions are selected from the group consisting of W16A, K22A, D27A, E29A, K31A, K34A, D42A and L68A.
46. The cell death inducing CAR according to any one of items 1 to 4, wherein the death domain comprises, or consists of, an amino acid sequence having at least 80% sequence identity with SEQ ID NO: 9, the amino acid sequence comprising at least one amino acid substitution at a position which corresponds to a position in SEQ ID NO: 9 selected from the group consisting of W16, R21, R22, L25, D27, E29, I30, D31, R32, E34, E43, W52, L63, R68 and L70.
47. The cell death inducing CAR according to any one of items 1 to 4, wherein the death domain comprises, or consists of, an amino acid sequence which differs from the amino acid sequence of SEQ ID NO: 9 in that one or more amino acid residues at positions selected from the group consisting of W16, R21, R22, L25, D27, E29, I30, D31, R32, E34, E43, W52, L63, R68 and L70 are substituted.
48. The cell death inducing CAR according to any one of items 1 to 4, wherein the death domain comprises, or consists of, an amino acid sequence having at least 80% sequence identity with SEQ ID NO: 9, the amino acid sequence comprising at least one amino acid substitution at a position which corresponds to a position in SEQ ID NO: 9 selected from the group consisting of W16, R22, D27, E29, D31, E34, E43 and L70.
49. The cell death inducing CAR according to item 48, wherein the at least one amino acid substitution is selected from the group consisting of W16A, R22A, D27A, E29A, D31A, E34A, E43A and L70A.
50. The cell death inducing CAR according to any one of items 1 to 4, wherein the death domain comprises, or consists of, an amino acid sequence which differs from the amino acid sequence of SEQ ID NO: 9 in that one or more amino acid residues at positions selected from the group consisting of W16, R22, D27, E29, D31, E34, E43 and L70 are substituted.
51. The cell death inducing CAR according to item 50, wherein the one or more substitutions are selected from the group consisting of W16A, R22A, D27A, E29A, D31A, E34A, E43A and L70A.
52. The cell death inducing CAR according to any one of items 1 to 4, wherein the death domain comprises, or consists of, an amino acid sequence having at least 80% sequence identity with SEQ ID NO: 10, the amino acid sequence comprising at least one amino acid substitution at a position which corresponds to a position in SEQ ID NO: 10 selected from the group consisting of W16, R21, T22, L25, E27, E29, I30, E31, E34, D42, W51, L59 and L66.
53. The cell death inducing CAR according to any one of items 1 to 4, wherein the death domain comprises, or consists of, an amino acid sequence which differs from the amino acid sequence of SEQ ID NO: 10 in that one or more amino acid residues at positions selected from the group consisting of W16, R21, T22, L25, E27, E29, I30, E31, E34, D42, W51, L59 and L66 are substituted.
54. The cell death inducing CAR according to any one of items 1 to 4, wherein the death domain comprises, or consists of, an amino acid sequence having at least 80% sequence identity with SEQ ID NO: 10, the amino acid sequence comprising at least one amino acid substitution at a position which corresponds to a position in SEQ ID NO: 10 selected from the group consisting of W16, T22, E27, E29, E31, E34, D42 and L66.
55. The cell death inducing CAR according to item 54, wherein the at least one amino acid substitution is selected from the group consisting of W16A, T22A, E27A, E29A, E31A, E34A, D42A and L66A.
56. The cell death inducing CAR according to any one of items 1 to 4, wherein the death domain comprises, or consists of, an amino acid sequence which differs from the amino acid sequence of SEQ ID NO: 10 in that one or more amino acid residues at positions selected from the group consisting of W16, T22, E27, E29, E31, E34, D42 and L66 are substituted.
57. The cell death inducing CAR according to item 56, wherein the one or more substitutions are selected from the group consisting of W16A, T22A, E27A, E29A, E31A, E34A, D42A and L66A.
58. The cell death inducing CAR according to item 1, wherein the endodomain including the at least one death domain is derived from the intracellular domain of Fas (CD95).
59. The cell death inducing CAR according to item 1 or 58, wherein the endodomain comprises, or consists of, an amino acid sequence having at least 80% sequence identity with SEQ ID NO: 11, the amino acid sequence comprising at least one amino acid substitution at a position which corresponds to a position in SEQ ID NO: 11 selected from the group consisting of V55, R60, K61, V64, E66, K68, I69, D70, E71, K73, E82, W91, Y101, K106 and L108.
60. The cell death inducing CAR according to item 1 or 58, wherein the endodomain comprises, or consists of, an amino acid sequence which differs from the amino acid sequence of SEQ ID NO: 11 in that one or more amino acid residues at positions selected from the group consisting of V55, R60, K61, V64, E66, K68, I69, D70, E71, K73, E82, W91, Y101, K106 and L108 are substituted.
61. The cell death inducing CAR according to item 1 or 58, wherein the endodomain comprises, or consists of, an amino acid sequence having at least 80% sequence identity with SEQ ID NO: 11, the amino acid sequence comprising at least one amino acid substitution at a position which corresponds to a position in SEQ ID NO: 11 selected from the group consisting of R60, V64, E66, D70, E71, K73, Y101 and K106.
62. The cell death inducing CAR according to item 61, wherein the at least one amino acid substitution is selected from the group consisting of R60A, V64N, E66A, D70A, E71A, K73A, Y101D and K106A.
63. The cell death inducing CAR according to item 61 or 62, wherein the at least one amino acid substitution includes K106A.
64. The cell death inducing CAR according to item 1 or 58, wherein the endodomain comprises, or consists of, an amino acid sequence which differs from the amino acid sequence of SEQ ID NO: 11 in that one or more amino acid residues at positions selected from the group consisting of R60, V64, E66, D70, E71, K73, Y101 and K106 are substituted.
65. The cell death inducing CAR according to item 64, wherein the one or more substitutions are selected from the group consisting of R60A, V64N, E66A, D70A, E71A, K73A, Y101D and K106A.
66. The cell death inducing CAR according to item 64 or 65, wherein the amino acid sequence includes the substitution K67A.
67. The cell death inducing CAR according to item 1, wherein the endodomain including the at least one death domain is derived from the intracellular domain of DR4.
68. The cell death inducing CAR according to item 1 or 67, wherein the endodomain comprises, or consists of, an amino acid sequence having at least 80% sequence identity with SEQ ID NO: 12, the amino acid sequence comprising at least one amino acid substitution at a position which corresponds to a position in SEQ ID NO: 12 selected from the group consisting of: W118, R123, Q124, L127, K129, E131, I132, D133, V134, R136, D144, W153, I163, D168 and L170.
69. The cell death inducing CAR according to item 1 or 67, wherein the endodomain comprises, or consists of, an amino acid sequence which differs from the amino acid sequence of SEQ ID NO: 12 in that one or more amino acid residues at positions selected from the group consisting of W118, R123, Q124, L127, K129, E131, I132, D133, V134, R136, D144, W153, I163, D168 and L170 are substituted.
70. The cell death inducing CAR according to item 1 or 67, wherein the endodomain comprises, or consists of, an amino acid sequence having at least 80% sequence identity with SEQ ID NO: 12, the amino acid sequence comprising at least one amino acid substitution at a position which corresponds to a position in SEQ ID NO: 12 selected from the group consisting of: W118, Q124, K129, E131, D133, R136, D144 and L170.
71. The cell death inducing CAR according to item 70, wherein the at least one amino acid substitution is selected from the group consisting of: W118A, Q124A, K129A, E131A, D133A, R136A, D144A and L170A.
72. The cell death inducing CAR according to item 1 or 67, wherein the endodomain comprises, or consists of, an amino acid sequence which differs from the amino acid sequence of SEQ ID NO: 12 in that one or more amino acid residues at positions selected from the group consisting of W118, Q124, K129, E131, D133, R136, D144 and L170 are substituted.
73. The cell death inducing CAR according to item 72, wherein the one or more substitutions are selected from the group consisting of W118A, Q124A, K129A, E131A, D133A, R136A, D144A and L170A.
74. The cell death inducing CAR according to item 1, wherein the endodomain including the at least one death domain is derived from the intracellular domain of DR5.
75. The cell death inducing CAR according to item 1 or 74, wherein the endodomain comprises, or consists of, an amino acid sequence having at least 80% sequence identity with SEQ ID NO: 13, the amino acid sequence comprising at least one amino acid substitution at a position which corresponds to a position in SEQ ID NO: 13 selected from the group consisting of: W123, R128, K129, L132, D134, E136, I137, K138, V139, K141, D149, W158, V168, D173 and L175.
76. The cell death inducing CAR according to item 1 or 74, wherein the endodomain comprises, or consists of, an amino acid sequence which differs from the amino acid sequence of SEQ ID NO: 13 in that one or more amino acid residues at positions selected from the group consisting of W123, R128, K129, L132, D134, E136, I137, K138, V139, K141, D149, W158, V168, D173 and L175 are substituted.
77. The cell death inducing CAR according to item 1 or 74, wherein the endodomain comprises, or consists of, an amino acid sequence having at least 80% sequence identity with SEQ ID NO: 13, the amino acid sequence comprising at least one amino acid substitution at a position which corresponds to a position in SEQ ID NO: 11 selected from the group consisting of: W123, K129, D134, E136, K138, K141, D149 and L175.
78. The cell death inducing CAR according to item 77, wherein the at least one amino acid substitution is selected from the group consisting of: W123A, K129A, D134A, E136A, K138A, K141A, D149A and L175A.
79. The cell death inducing CAR according to item 1 or 74, wherein the endodomain comprises, or consists of, an amino acid sequence which differs from the amino acid sequence of SEQ ID NO: 13 in that one or more amino acid residues at positions selected from the group consisting of W123, K129, D134, E136, K138, K141, D149 and L175 are substituted.
80. The cell death inducing CAR according to item 1 or 74, wherein the one or more substitutions are selected from the group consisting of W123A, K129A, D134A, E136A, K138A, K141A, D149A and L175A.
81. The cell death inducing CAR according to item 1, wherein the endodomain including the at least one death domain is derived from the intracellular domain of TNFR1.
82. The cell death inducing CAR according to item 1 or 81, wherein the endodomain comprises, or consists of, an amino acid sequence having at least 80% sequence identity with SEQ ID NO: 14, the amino acid sequence comprising at least one amino acid substitution at a position which corresponds to a position in SEQ ID NO: 14 selected from the group consisting of W137, R142, R143, L146, D148, E150, I151, D152, R153, E155, E43, W173, L184, R189 and L191.
83. The cell death inducing CAR according to item 1 or 81, wherein the endodomain comprises, or consists of, an amino acid sequence which differs from the amino acid sequence of SEQ ID NO: 14 in that one or more amino acid residues at positions selected from the group consisting of W137, R142, R143, L146, D148, E150, I151, D152, R153, E155, E164, W173, L184, R189 and L191 are substituted.
84. The cell death inducing CAR according to item 1 or 81, wherein the endodomain comprises, or consists of, an amino acid sequence having at least 80% sequence identity with SEQ ID NO: 14, the amino acid sequence comprising at least one amino acid substitution at a position which corresponds to a position in SEQ ID NO: 14 selected from the group consisting of W137, R143, D148, E150, D152, E155, E164 and L191.
85. The cell death inducing CAR according to item 84, wherein the at least one amino acid substitution is selected from the group consisting of W137A, R143A, D148A, E150A, D152A, E155A, E164A and L191A.
86. The cell death inducing CAR according to item 1 or 81, wherein the endodomain comprises, or consists of, an amino acid sequence which differs from the amino acid sequence of SEQ ID NO: 14 in that one or more amino acid residues at positions selected from the group consisting of W137, R143, D148, E150, D152, E155, E164 and L191 are substituted.
87. The cell death inducing CAR according to item 86, wherein the one or more substitutions are selected from the group consisting of W137A, R143A, D148A, E150A, D152A, E155A, E164A and L191A.
88. The cell death inducing CAR according to item 1, wherein the endodomain including the at least one death domain is derived from the intracellular domain of DR3.
89. The cell death inducing CAR according to item 1 or 88, wherein the endodomain comprises, or consists of, an amino acid sequence having at least 80% sequence identity with SEQ ID NO: 15, the amino acid sequence comprising at least one amino acid substitution at a position which corresponds to a position in SEQ ID NO: 15 selected from the group consisting of W127, R132, T133, L136, E138, E140, I141, E142, E145, D153, W162, L170 and L177.
90. The cell death inducing CAR according to item 1 or 88, wherein the endodomain comprises, or consists of, an amino acid sequence which differs from the amino acid sequence of SEQ ID NO: 15 in that one or more amino acid residues at positions selected from the group consisting of W127, R132, T133, L136, E138, E140, I141, E142, E145, D153, W162, L170 and L177 are substituted.
91. The cell death inducing CAR according to item 1 or 88, wherein the endodomain comprises, or consists of, an amino acid sequence having at least 80% sequence identity with SEQ ID NO: 15, the amino acid sequence comprising at least one amino acid substitution at a position which corresponds to a position in SEQ ID NO: 15 selected from the group consisting of W127, T133, E138, E140, E142, E145, D153 and L177.
92. The cell death inducing CAR according to item 91, wherein the at least one amino acid substitution is selected from the group consisting of W127A, T133A, E138A, E140A, E142A, E145A, D153A and L177A.
93. The cell death inducing CAR according to item 1 or 88, wherein the endodomain comprises, or consists of, an amino acid sequence which differs from the amino acid sequence of SEQ ID NO: 15 in that one or more amino acid residues at positions selected from the group consisting of W127, T133, E138, E140, E142, E145, D153 and L177 are substituted.
94. The cell death inducing CAR according to item 93, wherein the one or more substitutions are selected from the group consisting of W127A, T133A, E138A, E140A, E142A, E145A, D153A and L177A.
95. The cell death inducing CAR according to any one of items 1 to 94, wherein the hinge is derived from the extracellular domain of a transmembrane receptor of the tumor necrosis factor (TNF) superfamily death receptor.
96. The cell death inducing CAR according to any one of items 1 to 95 wherein the hinge is derived from the extracellular domain of CD95 (Fas).
97. The cell death inducing CAR according to any one of items 1 to 96, wherein the hinge comprises, or consists of, an amino acid sequence having at least 80% sequence identity with any one of SEQ ID NOs: 16 to 18.
98. The cell death inducing CAR according to any one of items 1 to 94, wherein the hinge is selected from group consisting of IgG1 hinge, CD8a hinge and FcγRIIIα hinge.
99. The cell death inducing CAR according to any one of items 1 to 98, wherein the at least one transmembrane domain is selected from the group consisting of CD95 (Fas) transmembrane domain, DR4 transmembrane domain, DR5 transmembrane domain, TNFR1 transmembrane domain, DR3 transmembrane domain, CD8 alpha transmembrane domain, 4-1BB transmembrane domain, DAP10 transmembrane domain and CD28 transmembrane domain.
100. The cell death inducing CAR according to any one of items 1 to 98, wherein the at least one transmembrane domain is derived from CD95 (Fas), such as human CD95.
101. The cell death inducing CAR according to any one of items 1 to 98, wherein the transmembrane domain is selected from the group consisting of the transmembrane domains of the FcεRI α, β and γ chains.
102. The cell death inducing CAR according to any one of items 1 to 101, wherein the transmembrane domain comprises one or more, such as 1 to 8, mutations in its amino acid sequence compared to the amino acid sequence of the wild type transmembrane domain from which it is derived, which mutation(s) attenuate(s) the self-association of the cell death inducing chimeric antigen receptor.
103. The cell death inducing CAR according to any one of items 1 to 102, wherein the transmembrane domain comprises one or more, such as 1 to 8, amino acid substitutions in its amino acid sequence compared to the amino acid sequence of the wild type transmembrane domain from which it is derived, which amino acid substitution(s) attenuate(s) the self-association of the cell death inducing chimeric antigen receptor.
104. The cell death inducing CAR according to any one of items 1 to 98, wherein the transmembrane domain comprises, or consists of, an amino acid sequence having at least 80% sequence identity with SEQ ID NO: 25, the amino acid sequence comprising at least one amino acid substitution at a position which corresponds to a position in SEQ ID NO: 25 selected from the group consisting of C5, L7, L7, P10, I11, P12, L13 and I14.
105. The cell death inducing CAR according to item 104, wherein the at least one amino acid substitution is selected from the group consisting of C5R, C5A, L7F, L7A, P10L, P10A, I11A, P12A, L13A and I14A.
106. The cell death inducing CAR according to any one of items 1 to 98, wherein the transmembrane domain comprises, or consists of, an amino acid sequence which differs from the amino acid sequence of SEQ ID NO: 25 in that one or more amino acid residues at positions selected from the group consisting of C5, L7, L7, P10, I11, P12, L13 and I14 are substituted.
107. The cell death inducing CAR according to item 106, wherein the at least one amino acid substitution is selected from the group consisting of C5R, C5A, L7F, L7A, P10L, P10A, I11A, P12A, L13A and I14A.
108. The cell death inducing CAR according to any one of items 1 to 107, wherein the extracellular ligand-binding domain is an extracellular antigen-binding domain.
109. The cell death inducing CAR according to any one of items 1 to 108, wherein the extracellular ligand-binding domain is specific for a cell surface antigen N, N being expressed on a non-pathological or healthy cell, but not being expressed on a targeted pathological (e.g., cancerous) cell.
110. The cell death inducing CAR according to any one of items 1 to 108, wherein the extracellular-ligand binding domain of the cell death inducing CAR is specific for a target antigen (e.g., cell surface antigen) selected from the group consisting of: CD123; CD19; CD22; CD30; CD70; CD171; CS-1 (also referred to as CD2 subset 1, CRACC, SLAMF7, CD319, and 19A24); DLL3; TSPAN10; PRAME; C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-I IRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1 (MUC1); Mucin 16 (MUC16); Mucin 17 (MUC17); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAFX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(I-4)bDGlcp(I-I)Cer); transglutaminase 5 (TGS5); high molecular weight-melanoma-associated antigen (HMWMAA); o-acetyl-GD2 (OAcGD2); Folate receptor beta; tumor endothelial marker 1 (TEM1/CD248); tumor endothelial marker 7-related (TEM7R); claudin 6 (CLDN6); claudin 18 (CLDN18), including splice variant 2 (claudin18.2); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Lymphocyte antigen 6 complex locus protein G6d (LY6G6D); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70 (HSP70); heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), CD56, CD205, CD83, CD206, CD200, CD36, RARRES1, Troponin C, Beta-1 integrin, CCKBR, GALR1, CD4, CD20, CD22, CD25, CD34, MUC1.
111. The cell death inducing CAR according to any one of items 1 to 108, wherein the extracellular ligand-binding domain is specific for an antigen selected from the group consisting of CD56, CD205, CD83, CD206, CD200, CD36, RARRES1, Troponin C, Beta-1 integrin, CCKBR, GALR1, CD4, CD20, CD22, CD25, CD34, MUC1 and EGFRVIII.
112. The cell death inducing CAR according to any one of items 1 to 108, wherein the extracellular ligand-binding domain is specific for CD20.
113. The cell death inducing CAR according to any one of items 1 to 112, which is a single chain CAR.
114. The cell death inducing CAR according to any one of items 1 to 112, which is a multi-chain CAR.
115. The cell death inducing CAR according to item 114, wherein the at least one ectodomain and the at least one endodomain of said CAR are not born on the same polypeptide chain, but on at least two different polypeptide chains each containing a transmembrane domain, said at least two different polypeptide chains interact to form a dimeric or a multimeric CAR.
116. The cell death inducing CAR according to item 115, wherein said at least two different polypeptide chains comprise a portion of a FcεRI alpha chain, FcεRI beta chain and/or FcεRI gamma chain.
117. The cell death inducing CAR according to item 115 or 116, wherein the polypeptide chain comprising the ectodomain comprises the transmembrane domain from the alpha chain of FcεRI.
118. The cell death inducing CAR according to any one of items 115 to 117, wherein the polypeptide chain comprising the endodomain comprising the at least one death domain comprises the transmembrane domain from the gamma or beta chain of FcεRI.
119. A polynucleotide comprising a nucleic acid sequence encoding a cell death inducing CAR according to any one of items 1 to 118.
120. A polynucleotide comprising nucleic acid sequences encoding two or more polypeptide chains composing the multi-chain CAR according to any one of items 115 to 117.
121. The polynucleotide according to items 119 or 120, wherein the cell death inducing CAR encoding nucleic acid sequence is operably linked to a promoter.
122. The polynucleotide according to any one of items 119 to 121, wherein the cell death inducing CAR encoding nucleic acid sequence is operably linked to a promoter selected from the group consisting of pUBC, pLCK, pEF1a short, pEF1a long, pGK1, pSFFV, pTCF7L1, pTCF7L2, pTCF7, and derivatives of any of the aforesaid.
123. The polynucleotide according to any one of items 119 to 121, wherein the cell death inducing CAR encoding nucleic acid sequence is operably linked to a promoter selected from the group consisting of pUBC, pLCK, pEF1a short, pGK1, pTCF7L1, pTCF7L2, pTCF7 and derivatives of any of the aforesaid.
124. The polynucleotide according to any one of items 119 to 121, wherein the cell death inducing CAR encoding nucleic acid sequence is operably linked to the promoter pGK1 or a derivative thereof, such as a derivative selected from the group consisting of pGK100, pGK200, pGK300 and pGK400.
125. The polynucleotide according to any one of items 119 to 121, wherein the cell death inducing CAR encoding nucleic acid sequence is operably linked to a pEF1a short promoter.
126. A vector, such as an expression vector, comprising the polynucleotide according to any one of items 119 to 125.
127. A stem cell or cell derived therefrom comprising (such as expressing at its surface) at least one cell death inducing CAR according to any one of items 1 to 118.
128. A stem cell or cell derived therefrom comprising the polynucleotide according to any one of items 119 to 125 or the vector according to item 126.
129. The stem cell or cell derived therefrom according to item 127 or 128, further comprising (such as expressing at its surface) an activating CAR.
130. The stem cell or cell derived therefrom according to any one of items 127 to 129, further comprising a polynucleotide comprising a nucleic acid sequence encoding an activating CAR operably linked to a promoter.
131. The stem cell or cell derived therefrom according to item 129 or 130, wherein the activating CAR comprises
   a) at least one ectodomain which comprises an extracellular ligand-binding domain;
   b) at least one transmembrane domain; and
   c) at least one endodomain which comprises a signal transducing domain and optionally a co-stimulatory domain.
132. The stem cell or cell derived therefrom according to item 131, wherein the extracellular ligand-binding domain of the activating CAR is specific for a cell surface antigen P, P being expressed or over expressed on a targeted pathological (e.g., cancerous) cell.
133. The stem cell or cell derived therefrom according to item 131 or 132, wherein the extracellular-ligand binding domain of the activating CAR is specific for a target antigen (e.g., cell surface antigen) selected from the group consisting of: CD123; CD19; CD22; CD30; CD70; CD171; CS-1 (also referred to as CD2 subset 1, CRACC, SLAMF7, CD319, and 19A24); DLL3; TSPAN10; PRAME; C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-I IRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1 (MUC1); Mucin 16 (MUC16); Mucin 17 (MUC17); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAFX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(I-4)bDGlcp(I-I)Cer); transglutaminase 5 (TGS5); high molecular weight-melanoma-associated antigen (HMWMAA); o-acetyl-GD2 (OAcGD2); Folate receptor beta; tumor endothelial marker 1 (TEM1/CD248); tumor endothelial marker 7-related (TEM7R); claudin 6 (CLDN6); claudin 18 (CLDN18), including splice variant 2 (claudin18.2); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Lymphocyte antigen 6 complex locus protein G6d (LY6G6D); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70 (HSP70); heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).
134. The stem cell or cell derived therefrom according to any one of items 131 to 133, wherein the extracellular ligand-binding domain of the activating CAR is specific for a target antigen selected from the group consisting of CD123, ROR1, BCMA, PSMA, CD33, CD38, CD22, CS1, CLL-1, HSP70, EGFRVIII, FLT3, WT1, CD30, CD70, MUC1, MUC16, MUC17, PRAME, TSPAN10, Claudin18.2, DLL3, LY6G6D and GD2 (including O-acetyl-GD2).
135. The stem cell or cell derived therefrom according to any one of items 127 to 134, wherein the cell is a hematopoietic stem cell.
136. The stem cell or cell derived therefrom according to any one of items 127 to 135, wherein the cell is a lymphoid cell.
137. The stem cell or cell derived therefrom according to any one of items 127 to 136, which is a human cell.
138. The stem cell or cell derived therefrom according to any one of items 127 to 137, wherein the gene(s) encoding beta 2-microglobulin (B2M) and/or class II major histocompatibility complex transactivator (CIITA) has (have) been inactivated.
139. The stem cell or cell derived therefrom according to any one of items 127 to 138, wherein least one gene encoding a component of the T-cell receptor (TCR) has been inactivated.
140. The stem cell or cell derived therefrom according to any one of items 127 to 139, wherein said cell has been modified to confer resistant to at least one immune suppressive drug, chemotherapy drug, or anti-cancer drug.
141. An immune cell comprising (such as expressing at its surface) at least one cell death inducing CAR according to any one of items 1 to 118.
142. An immune cell comprising the polynucleotide according to any one of items 119 to 125 or the vector according to item 126.
143. The immune cell according to item 142, wherein the immune cell expresses at its surface the cell death inducing receptor encoded by the polynucleotide.
144. The immune cell according to any one of items 141 to 143, further comprising (such as expressing at its surface) an activating CAR.
145. The immune cell according to any one of items 141 to 144, further comprising a polynucleotide comprising a nucleic acid sequence encoding an activating CAR operably linked to a promoter, such as a native promoter.
146. The immune cell according to item 144 or 145, wherein the activating CAR comprises
   a) at least one ectodomain which comprises an extracellular ligand-binding domain;
   b) at least one transmembrane domain; and
   c) at least one endodomain which comprises a signal transducing domain and optionally a co-stimulatory domain.
147. The immune cell according to item 146, wherein the extracellular ligand-binding domain of the activating CAR is specific for a cell surface antigen P, P being expressed or over expressed on a targeted pathological (e.g., cancerous) cell.
148. The immune cell according to item 146 or 147, wherein the extracellular-ligand binding domain of the activating CAR is specific for a target antigen (e.g., cell surface antigen) selected from the group consisting of: CD123; CD19; CD22; CD30; CD70; CD171; CS-1 (also referred to as CD2 subset 1, CRACC, SLAMF7, CD319, and 19A24); DLL3; TSPAN10; PRAME; C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-I IRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1 (MUC1); Mucin 16 (MUC16); Mucin 17 (MUC17); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAFX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(I-4)bDGlcp(I-I)Cer); transglutaminase 5 (TGS5); high molecular weight-melanoma-associated antigen (HMWMAA); o-acetyl-GD2 (OAcGD2); Folate receptor beta; tumor endothelial marker 1 (TEM1/CD248); tumor endothelial marker 7-related (TEM7R); claudin 6 (CLDN6); claudin 18 (CLDN18), including splice variant 2 (claudin18.2); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Lymphocyte antigen 6 complex locus protein G6d (LY6G6D); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70 (HSP70); heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).
149. The immune cell according to any one of items 146 to 148, wherein the extracellular ligand-binding domain of the activating CAR is specific for a target antigen selected from the group consisting of CD123, ROR1, BCMA, PSMA, CD33, CD38, CD22, CS1, CLL-1, HSP70, EGFRVIII, FLT3, WT1, CD30, CD70, MUC1, MUC16, MUC17, PRAME, TSPAN10, Claudin18.2, DLL3, LY6G6D and GD2 (including O-acetyl-GD2).
150. The immune cell according to any one of items 141 to 149, wherein said immune cell is a T cell.
151. The immune cell according to any one of items 141 to 150, wherein said immune cell is a primary T cell.
152. The immune cell according to any one of items 141 to 151, wherein said immune cell is a virus-specific T cell (VST), preferably isolated from a donor.
153. The immune cell according to any one of items 141 to 152, wherein said immune cell is derived from an inflammatory T-lymphocyte, cytotoxic T-lymphocyte, regulatory T-lymphocyte, tumor infiltrating lymphocyte or helper T-lymphocyte.
154. The immune cell according to any one of items 141 to 152, which is derived from a cytotoxic T-lymphocyte.
155. The immune cell according to any one of items 141 to 154, which is a human cell.
156. The immune cell according to any one of items 141 to 155, wherein the gene(s) encoding beta 2-microglobulin (B2M) and/or class II major histocompatibility complex transactivator (CIITA) has (have) been inactivated.
157. The immune cell according to any one of items 141 to 156, wherein at least one gene encoding a component of the T-cell receptor (TCR) has been inactivated.
158. The immune cell according to any one of items 141 to 157, wherein said cell has been modified to confer resistant to at least one immune suppressive drug, chemotherapy drug, or anti-cancer drug.
159. A population of cells according to any one of items 127 to 140.
160. A population of immune cells according any one of items 141 to 158.
161. The stem cell or cell derived therefrom according to any one of items 127 to 140 or the population according to item 159 for use as a medicament.
162. The immune cell according to any one of items 141 to 158 or the population according to item 160 for use as a medicament.
163. The stem cell or cell derived therefrom according to any one of items 127 to 140 or the population according to item 159 for use in the treatment of a cancer or viral infection.
164. The immune cell according to any one of items 141 to 158 or the population according to item 160 for use in the treatment of a cancer or viral infection.
165. The stem cell or cell derived therefrom according to any one of items 127 to 140 or the population according to item 159 for use in the treatment of a solid tumor.
166. The immune cell according to any one of items 141 to 158 or the population according to item 160 for use in the treatment of a solid tumor.
167. The stem cell or cell derived therefrom according to any one of items 127 to 140 or the population according to item 159 for use in the treatment of a B cell malignancy.
168. The immune cell according to any one of items 141 to 158 or the population according to item 160 for use in the treatment of a B cell malignancy.
169. A method for treating a patient in need thereof comprising:
   a) Providing cells according to any one of items 127 to 140;
   b) Administrating said cells to said patient.
170. A method for treating a patient in need thereof comprising:
   a) Providing immune cells according to any one of items 141 to 158;
   b) Administrating said immune cells to said patient.
171. The method according to item 169 or 170, wherein said cells under a) are recovered from donors (allogeneic mode).
172. The method according to item 169 or 170, wherein said cells under a) are recovered from the patient in need thereof (autologous mode).
173. A method for engineering a stem cell or cell derived therefrom, said method comprises:
   (a) Providing a stem cell or cell derived therefrom;
   (b) Introducing into said cell at least one polynucleotide according to any one of items 119 to 125, or a vector according to item 126; and
   (c) Expressing said cell death inducing chimeric antigen receptor in said cell. 174. A method for engineering an immune cell, said method comprises:
   (a) Providing an immune cell;
   (b) Introducing into said immune cell at least one polynucleotide according to any one of items 119 to 125, or a vector according to item 126; and
   (c) Expressing said cell death inducing chimeric antigen receptor in said cell.
175. The method according to item 173 or 174, further comprising (d) Introducing into said immune cell at least one polynucleotide or vector encoding an activating chimeric antigen receptor; and (e) Expressing said activating chimeric antigen receptor in said cell.
176. The method according to item 175, wherein the activating CAR is as defined in any one of items 131 to 134 and 146 to 149, respectively.
177. The method according to item 174 or 175, wherein step (d) is performed after step (a) and before step (c).
178. The method according to any one items 173 to 177, further comprising at least one step of editing (e.g., inactivating) at least one gene selected from TCR encoding genes, immune check point genes, genes involved in drug resistance, and combinations thereof.
179. The method according to any one items 173 to 178, further comprising at least one step of inactivating at least one gene selected from the group consisting of B2M gene, CIITA gene, CD52 gene, GR gene, TCR alpha gene, TCR beta gene, HLA gene, immune check point genes such as PD1 gene and CTLA-4 gene, drug sensitizing genes, such as the dCK gene and HPRT gene, and drug resistance genes.
180. The method according to any one items 173 to 178, further comprising at least one step of inactivating a TCR alpha gene, TCR beta gene, CD52 gene and/or dCK gene.

### BRIEF DESCRIPTION OF DRAWINGS

FIG. 1: Non-limiting schematic representation of the design of cell death inducing Chimeric Antigen Receptors: the native death receptor is engineered by replacing the native extracellular topological domain by an extracellular ligand binding domain able to bind specifically to an antigen or cell surface marker of, e.g., an “off-target” healthy cell.

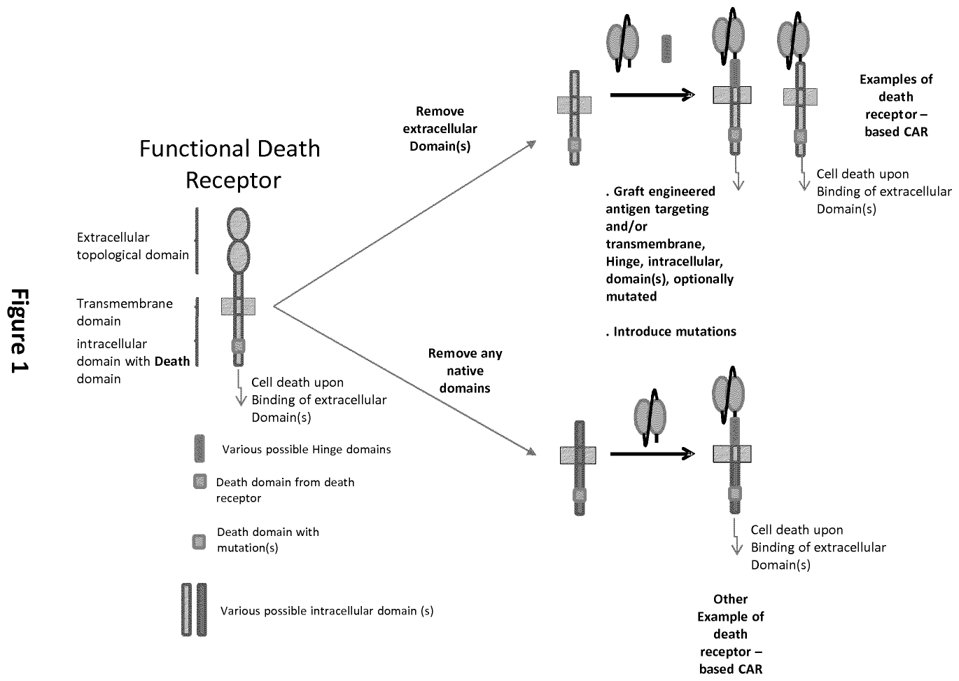

FIG. 2: Percentage of Jurkat cells positive for the activation of the caspase 3/7. Jurkat cells transduced with lentiviral particles encoding the different cell death inducing CARs, are co-incubated with target cell lines expressing either the cell death inducing CAR target antigen (CD19 expressing HEK293, black bars) or a non-relevant antigen (PSMA expressing HEK293, white bars). The activation of the caspase 3/7 is monitored using the CellEvent Caspase-3/7 Green Flow Cytometry Assay Kit.

FIG. 3: Percentage of BFP (reporter of cell death inducing CAR expression) positive T-cells. Primary T-cells transduced with lentiviral particles encoding the different FAS-based cell death inducing CARs, are co-incubated with target cell lines expressing either the cell death inducing CAR target antigen (CD19 expressing HEK293, black bars) or a non-relevant antigen (PSMA expressing HEK293, white bars). The percentage of BFP positive cells is measured by flow cytometry. The percentage of BFP positive cell is normalized to the one measured in absence of target cells.

FIG. 4: Percentage of BFP (reporter of cell death inducing CAR expression) positive T-cells. Primary T-cells transduced with lentiviral particles encoding the different FAS-based cell death inducing CARs, are cultures for up to 17 days post activation/transduction with a reactivation with beads at day 14. The percentage of BFP positive cells is measured by flow cytometry and normalized to the one measured at day 5.

FIG. 5: Percentage of EGFP (reporter of cell death inducing CAR expression) positive T-cells. Primary T-cells transduced with lentiviral particles encoding the DR4 and DR5-based cell death inducing CARs, are co-incubated with target cell lines expressing either the cell death inducing CAR target antigen (PSMA expressing HEK293, black bars) or a non-relevant antigen (CD19 expressing HEK293, white bars). The percentage of EGFP positive cells is measured by flow cytometry. The percentage of EGFP positive cell is normalized to the one measured in absence of target cells.

### DETAILED DESCRIPTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Cell Death Inducing CAR According to the Invention

The present invention thus provides a cell death inducing chimeric antigen receptor which comprises at least one death domain (such as at least two death domains) in its endodomain. More particularly, the present invention provides a cell death inducing chimeric antigen receptor (CAR) which comprises:
a) at least one ectodomain which comprises an extracellular ligand-binding domain and, optionally, a hinge;
b) at least one transmembrane domain; and
c) at least one endodomain which comprises at least one death domain.

The death domain may be any protein interaction domain which is capable of transmitting a death signal from the cell surface to the intracellular signalling pathway leading to the death of a cell. The death domain may be a protein interaction domain that has (or has retained) the capacity to cause DISC assembly upon ligand binding, leading to a subsequent caspase-8 activation. Preferably, the death domain interacts with one or more pro-apoptotic adaptor proteins involved in the extrinsic apoptosis pathway, such as FADD and TRADD.

In the present invention, a cell death inducing CAR (D-CAR) is preferably an apoptosis inducing CAR (apoCAR).

According to certain embodiments, the death domain is derived from a death receptor, such as a human death receptor.

According to certain embodiments, the death domain is derived from a death receptor selected from the group consisting of Fas (CD95); DR4; DR5; TNFR1; DR3; Leucine-rich repeat and death domain-containing protein 1, Ankyrin repeat and death domain-containing protein 1B, Interleukin-1 receptor-associated kinase-like 2, Tumor necrosis factor receptor superfamily member 21, Netrin receptor UNCSA, Netrin receptor UNCSB, Netrin receptor UNCSC, Netrin receptor UNCSD, UNCSC-like protein, Tumor necrosis factor receptor superfamily member 16, Ankyrin-1, Ankyrin-2, Ankyrin-3, Nuclear factor NF-kappa-B p105 subunit, Tumor necrosis factor receptor superfamily member 6, Interleukin-1 receptor-associated kinase 1, Death-associated protein kinase 1, Death domain-containing protein CRADD, Nuclear factor NF-kappa-B p100 subunit, FAS-associated death domain protein, Receptor-interacting serine/threonine-protein kinase 1, Ankyrin repeat and death domain-containing protein 1A, Death domain-containing protein 1, Ectodysplasin-A receptor-associated adapter protein, Tumor necrosis factor receptor superfamily member 25, THO complex subunit 1, Myeloid differentiation primary response protein MyD88, p53-induced death domain-containing protein 1, and Interleukin-1 receptor-associated kinase 3.

According to certain embodiments, the death domain is derived from a death domain of a transmembrane receptor of the tumor necrosis factor (TNF) superfamily, such as a human transmembrane receptor of the tumor necrosis factor (TNF) superfamily.

According to certain embodiment, the death domain is derived from a death domain of a member of the TNFR superfamily selected from the group consisting of: Fas (CD95), DR4, DR5, TNFR1 and DR3.

According to certain embodiments, the death domain is derived from the death domain of Fas (CD95), such as human Fas (CD95) (SEQ ID NO: 1).

According to certain embodiments, the death domain is derived from the death domain of DR4, such as human DR4 (SEQ ID NO: 2).

According to certain embodiments, the death domain is derived from the death domain of DR5, such as human DR5 (SEQ ID NO: 3).

According to certain embodiments, the death domain is derived from the death domain of TNFR1, such as human TNFR1 (SEQ ID NO: 4).

According to certain embodiments, the death domain is derived from the death domain of DR3, such as human DR3 (SEQ ID NO: 5).

Exemplary amino acid sequences of the death domains derived from Fas (CD95), DR4, DR5, TNFR1 and DR3 are set forth in SEQ ID NOs: 6, 7, 8, 9 and 10, respectively. The amino acid sequence of the death domain of Fas (CD95) as set forth in SEQ ID NO: 6 corresponds to amino acids 230 to 314 of the full length amino acid sequence of Fas as set forth in SEQ ID NO: 1. The amino acid sequence of the death domain of DR4 as set forth in SEQ ID NO: 7 corresponds to amino acids 365 to 448 of the full length amino acid sequence of DR4 as set forth in SEQ ID NO: 2. The amino acid sequence of the death domain of DR5 as set forth in SEQ ID NO: 8 corresponds to amino acids 339 to 422 of the full length amino acid sequence of DR5 as set forth in SEQ ID NO: 3. The amino acid sequence of the death domain of TNFR1 as set forth in SEQ ID NO: 9 corresponds to amino acids 356 to 441 of the full length amino acid sequence of TNFR1 as set forth in SEQ ID NO: 4. The amino acid sequence of the death domain of DR3 as set forth in SEQ ID NO: 10 corresponds to amino acids 332 to 413 of the full length amino acid sequence of DR3 as set forth in SEQ ID NO: 5.

According to certain embodiments, the death domain comprises, or consists of, an amino acid sequence having at least 80%, such as at least 85%, sequence identity with any one of the amino acid sequences set forth in SEQ ID NO: 6, 7, 8, 9 or 10.

According to certain embodiments, the death domain comprises, or consists of, an amino acid sequence having at least 85%, such as at least 90%, sequence identity with any one of the amino acid sequences set forth in SEQ ID NO: 6, 7, 8, 9 or 10.

According to certain embodiments, the death domain comprises, or consists of, an amino acid sequence having at least 90%, such as at least 95%, sequence identity with any one of the amino acid sequences set forth in SEQ ID NO: 6, 7, 8, 9 or 10.

According to certain embodiments, the death domain comprises, or consists of, an amino acid sequence having at least 80%, such as at least 85%, sequence identity with SEQ ID NO: 6.

According to certain embodiments, the death domain comprises, or consists of, an amino acid sequence having at least 85%, such as at least 90%, sequence identity with SEQ ID NO: 6.

According to certain embodiments, the death domain comprises, or consists of, an amino acid sequence having at least 90%, such as at least 95%, sequence identity with SEQ ID NO: 6.

According to certain embodiments, the death domain comprises, or consists of, an amino acid sequence having at least 80%, such as at least 85%, sequence identity with SEQ ID NO: 7.

According to certain embodiments, the death domain comprises, or consists of, an amino acid sequence having at least 85%, such as at least 90%, sequence identity with SEQ ID NO: 7.

According to certain embodiments, the death domain comprises, or consists of, an amino acid sequence having at least 90%, such as at least 95%, sequence identity with SEQ ID NO: 7.

According to certain embodiments, the death domain comprises, or consists of, an amino acid sequence having at least 80%, such as at least 85%, sequence identity with SEQ ID NO: 8.

According to certain embodiments, the death domain comprises, or consists of, an amino acid sequence having at least 85%, such as at least 90%, sequence identity with SEQ ID NO: 8.

According to certain embodiments, the death domain comprises, or consists of, an amino acid sequence having at least 90%, such as at least 95%, sequence identity with SEQ ID NO: 8.

According to certain embodiments, the death domain comprises, or consists of, an amino acid sequence having at least 80%, such as at least 85%, sequence identity with SEQ ID NO: 9.

According to certain embodiments, the death domain comprises, or consists of, an amino acid sequence having at least 85%, such as at least 90%, sequence identity with SEQ ID NO: 9.

According to certain embodiments, the death domain comprises, or consists of, an amino acid sequence having at least 90%, such as at least 95%, sequence identity with SEQ ID NO: 9.

According to certain embodiments, the death domain comprises, or consists of, an amino acid sequence having at least 80%, such as at least 85%, sequence identity with SEQ ID NO: 10.

According to certain embodiments, the death domain comprises, or consists of, an amino acid sequence having at least 85%, such as at least 90%, sequence identity with SEQ ID NO: 10.

According to certain embodiments, the death domain comprises, or consists of, an amino acid sequence having at least 90%, such as at least 95%, sequence identity with SEQ ID NO: 10.

According to certain embodiments, the endodomain including the at least one death domain is derived from a death receptor, such as a human death receptor.

According to certain embodiments, the endodomain including the at least one death domain is derived from the intracellular domain of a death receptor, such as a human death receptor.

According to certain embodiments, the endodomain including the at least one death domain is derived from a transmembrane receptor of the tumor necrosis factor (TNF) superfamily, such as a human transmembrane receptor of the tumor necrosis factor (TNF) superfamily.

According to certain embodiments, the endodomain including the at least one death domain is derived from the intracellular domain of a transmembrane receptor of the tumor necrosis factor (TNF) superfamily, such as a human transmembrane receptor of the tumor necrosis factor (TNF) superfamily.

According to certain embodiments, the endodomain including the at least one death domain is derived from a member of the TNFR superfamily selected from the group consisting of: Fas (CD95), DR4, DR5, TNFR1 and DR3.

According to certain embodiments, the endodomain including the at least one death domain is derived from the intracellular domain of a member of the TNFR superfamily selected from the group consisting of: Fas (CD95), DR4, DR5, TNFR1 and DR3.

According to certain embodiments, the endodomain including the at least one death domain is derived from the intracellular domain of Fas (CD95), such as human Fas (CD95) (SEQ ID NO: 11).

According to certain embodiments, the endodomain including the at least one death domain is derived from the intracellular domain of DR4, such as human DR4 (SEQ ID NO: 12).

According to certain embodiments, the endodomain including the at least one death domain is derived from the intracellular domain of DR5, such as human DR5 (SEQ ID NO: 13).

According to certain embodiments, the endodomain including the at least one death domain is derived from the intracellular domain of TNFR1, such as human TNFR1 (SEQ ID NO: 14).

According to certain embodiments, the endodomain including the at least one death domain is derived from the intracellular domain of DR3, such as human DR3 (SEQ ID NO: 15).

According to certain embodiments, the endodomain comprises, or consists of, an amino acid sequence having at least 80%, such as at least 85%, sequence identity with any one of the amino acid sequences set forth in SEQ ID NO: 11, 12, 13, 14 or 15.

According to certain embodiments, the endodomain comprises, or consists of, an amino acid sequence having at least 90%, such as at least 95%, sequence identity with any one of the amino acid sequences set forth in SEQ ID NO: 11, 12, 13, 14 or 15.

According to certain embodiments, the endodomain comprises, or consists of, an amino acid sequence having at least 80%, such as at least 85%, sequence identity with SEQ ID NO: 11.

According to certain embodiments, the endodomain comprises, or consists of, an amino acid sequence having at least 90%, such as at least 95%, sequence identity with SEQ ID NO: 11.

According to certain embodiments, the endodomain comprises, or consists of, an amino acid sequence having at least 80%, such as at least 85%, sequence identity with SEQ ID NO: 12.

According to certain embodiments, the endodomain comprises, or consists of, an amino acid sequence having at least 90%, such as at least 95%, sequence identity with SEQ ID NO: 12.

According to certain embodiments, the endodomain comprises, or consists of, an amino acid sequence having at least 80%, such as at least 85%, sequence identity with SEQ ID NO: 13.

According to certain embodiments, the endodomain comprises, or consists of, an amino acid sequence having at least 80%, such as at least 85%, sequence identity with SEQ ID NO: 14.

According to certain embodiments, the endodomain comprises, or consists of, an amino acid sequence having at least 90%, such as at least 95%, sequence identity with SEQ ID NO: 14.

According to certain embodiments, the endodomain comprises, or consists of, an amino acid sequence having at least 80%, such as at least 85%, sequence identity with SEQ ID NO: 15.

According to certain embodiments, the endodomain comprises, or consists of, an amino acid sequence having at least 90%, such as at least 95%, sequence identity with SEQ ID NO: 15.

According to certain embodiments, the cell death inducing CAR of the invention is a death receptor, such as Fas, wherein the native extracellular ligand-binding domain of said death receptor has been replaced by an extracellular ligand binding domain which is different from said naturally occurring extracellular ligand-binding domain.

According to certain embodiments, the cell death inducing CAR of the invention is a death receptor, such as Fas, wherein the native extracellular topological domain of said death receptor has been replaced by an extracellular ligand binding domain able to bind specifically to an antigen or cell surface marker of, e.g., an “off-target” healthy cell.

According to certain embodiments, the cell death inducing CAR of the invention comprises an intracellular domain, a transmembrane domain, and optionally a part of the extracellular domain all derived from a death receptor, such as Fas, fused to at least one extracellular ligand binding domain specific for an antigen.

Because of the apoptotic potential conferred by a wild type death domain, unspecific basal cell death might still be seen even in absence of a target for which the cell death inducing CAR shows specificity, which will have an effect on the viability of the immune cells endowed with such cell death inducing CAR. Particularly, the over-expression of a cell death inducing receptor may lead to non-ligand induced multimerization of the receptor and subsequent activation of the cell death signalling pathway. Alternatively, this over-expression may also lead to non-ligand induced interactions with downstream effectors of the cell death signalling pathway, both ultimately leading to the cell death. In the present case this is reflected by the percentage of immune cells that are positive for the expression of cell death inducing CAR. The viability of immune cells may however be maintained or even improved (less basal cell death) by attenuating the self-association of the receptor via the death domain and/or transmembrane domain. Similarly, the apoptotic potential may be reduced by attenuating the binding of the death domain to the pro-apoptotic adaptor protein with which it interacts within the apoptotic signalling pathway, such as FADD or TRADD. In other words, a cell death inducing CAR can be rendered less toxic to the immune cells in the absence of the ligand-induced cell death signal.

Residues in death domains of receptors of the tumor necrosis factor (TNF) superfamily which might play a critical for apoptosis signaling have already been described (Scott et al., 2009, Nature, 457(7232): 1019-1022; McDonald et al., 2001, J Biol Chem, 276(18): 14939-14945).

The inventors of the present invention have now identified and tested beneficial mutations, and more specifically amino acid substitutions, within a number of naturally occurring (wild-type) death domains which attenuate self-association of the receptor and/or binding to a pro-apoptotic or pro-necrotic adaptor protein, such as FADD or TRADD. The mutations counter-act the effect of non-ligand induced interactions on the cell viability, overall leading to a higher population of cell death inducing CAR positive cells.

Generally, a mutation, such as an amino acid substitution, in the at least one death domain is considered as being beneficial if it permits a target specific elimination in range with its wild type (unmutated) counterpart and if it improves the viability (less basal cell death) of an cell death inducing CAR positive cell population overtime in the absence of cell death inducing CAR target cells when compared to its wild type (unmutated) counterpart. In this respect, immune cells, such as primary T-cells, expressing the cell death inducing CAR are tested for their ability to be eliminated by target cells presenting the cell death inducing CAR target antigen. In addition, the viability (cell count) of the cell death inducing CAR positive population is followed over time (up to 20 days).

Therefore, according to certain embodiments, a cell death inducing chimeric antigen receptor is provided wherein the death domain comprises one or more (such as two or more), such as 1 to 15 (e.g., 1, 2, 3, 4, 5, 6, 7, 8, 9, 10, 11, 12, 13, 14 or 15), mutations in its amino acid sequence compared to the amino acid sequence of the wild type death domain from which it is derived, which mutation(s) attenuate(s) the self-association and/or binding to a pro-apoptotic adaptor protein.

According to certain embodiments, the death domain comprises one or more (such as two or more), such as 1 to 15 (e.g., 1, 2, 3, 4, 5, 6, 7, 8, 9, 10, 11, 12, 13, 14 or 15), amino acid substitutions, preferably non-conservative amino acid substitutions, in its amino acid sequence compared to the amino acid sequence of the wild type death domain from which it is derived, which amino acid substitution(s) attenuate(s) the self-association and/or binding to a pro-apoptotic adaptor protein.

Attenuate self-association and/or binding to a pro-apoptotic adaptor protein as referred to herein can be determined by assaying immune cells, such as primary T-cells, transduced with a cell death inducing CAR or a cell death inducing CAR containing a given mutation or a set of mutations, such as a given substitution or set of substitutions, for target antigen independent activation of the cell death. A given mutation, such as a given substitution, results in attenuated self-association and/or binding to pro-apoptotic adaptor proteins (e.g., FADD or TRADD) if the detection of a marker of cell death activation is reduced when compared to the wild type domain from which it is derived.

Commercially available kits have been developed to detect apoptotic, necrotic, and dead cells. Such kit rely, on uptake of viability dyes and surface labeling of specific markers via, inter alia, flow cytometry (non-limiting examples of commercially available kits: Miltenyi #130-092-052, ebioscience #88-8006-72, abcam #ab14085, BD biosciences #556547). Annexin V, a member of calcium-dependent phospholipid-binding proteins, binds to phosphatidylserine (PS). In healthy cells, PS are mainly located on the cytosolic side of the plasma membrane. Upon initiation of apoptosis, PS are translocated to the extracellular membrane, where they become accessible to Annexins. During late-stage apoptosis (and not the early stages of apoptosis), loss of cell membrane allows uptake of various viability dyes such as propidium iodide (PI), 7-AAD, eFluor660 or eFluor780 while allowing Annexin V binding to cytosolic PS. Annexin V staining will therefore pairs with viability dye staining. Therefore, Annexin V binding and uptake of viability dyes can be combined to monitoring the progression of apoptosis using flow cytometry.

A suitable method for detect apoptotic, necrotic, and dead cells using Annexin V and PI is as follows (96 well plate format): 1) 100 μl of the appropriate Annexin V 1× Binding Buffer (e.g., Annexin V Binding Buffer Miltenyi #130-092-820 for the Annexin V-FITC Kit Miltenyi #130-092-052) is added to the wells (cell concentration(s) are adjusted at the start of the experiment, e.g. co-culture with targets cells). 2) Wells are centrifuge at 300×g for 10 minutes. 3) Supernatant is aspirated. 4) Cell pellets are resuspended in 100 μl of the appropriate Annexin V 1× Binding Buffer (e.g., Annexin V Binding Buffer Miltenyi #130-092-820 for the Annexin V-FITC Kit Miltenyi #130-092-052). 5) 10 μl of an appropriate dilution (according to the manufacturer recommendations) of the labeled Annexin V (e.g., Annexin V-FITC Kit Miltenyi #130-092-052) is added. 6) Wells are incubated for 15-20 minutes in the dark at 4° C. or room temperature (according to the manufacturer recommendations). 7) Cells are washed with 100 μl of 1× annexin buffer (e.g., Annexin V Binding Buffer Miltenyi #130-092-820 for the Annexin V-FITC Kit Miltenyi #130-092-052). 8) Wells are centrifuge at 300×g for 5 minutes. 8) Supernatant is aspirated. 9) 4) Cell pellets are resuspended in 100 μl of the appropriate buffer (e.g., Annexin V Binding Buffer Miltenyi #130-092-820) and 1 μL of PI solution (e.g., Annexin V-FITC Kit Miltenyi #130-092-052) is added immediately prior to analysis by flow cytometry.

A suitable method for detecting apoptotic, necrotic, and dead cells using eFluor780 viability dye is as follows: 1) 100 μl of PBS is added to the wells (cell concentration(s) are adjusted at the start of the experiment, e.g. co-culture with targets cells). 2) Wells are centrifuge at 300×g for 5 minutes. 3) Supernatant is aspirated. 4) Cell pellets are resuspended in an appropriate volume (e.g. 50 μl) of an adequate dilution (according to the manufacturer recommendations) of eFlour780 in PBS. 5) Wells are incubated for 15-20 minutes in the dark at 4° C. or room temperature (according to the manufacturer recommendations). 6) 100 μl of PBS-2% FBS is added to the wells. 7) Wells are centrifuge at 300×g for 5 minutes. 8) Supernatant is aspirated. 9) Cell pellets is resuspended in an appropriate buffer according to subsequent labeling or analysis by flow cytometry.

Alternatively, commercially available kits have been developed to detect apoptotic cells through detection of caspase activation (e.g. caspase 3, caspase 7 and caspase 8). Such kits rely on the cleavage of a labeled caspase substrate and subsequent detection of the cleaved product in a colorimetric assay (e.g., abcam #ab39401 or ab39700), a luminescent assay (e.g., Promega #G8090) or in a flow cytometry assay (e.g., Thermo Fisher #C10427 or # C10747) as non-limiting examples. Caspase 3/7 activation and uptake of viability dyes can also be combined to monitoring the progression of apoptosis using flow cytometry. Below a detailed description of two protocols is provided allowing the detection of apoptotic cells.

A suitable method for detect apoptotic cells through detection of caspase activation (e.g. caspase 3, caspase 7 and caspase 8) is as follows (fluorescent labelling using caspase 3/7 reagents): 1) Wells are centrifuge at 300×g for 5 minutes (cell concentration(s) are adjusted at the start of the experiment, e.g. co-culture with targets cells). 2) Supernatant is aspirated. 3) Cell pellet is resuspended in 100 μl of appropriate growth medium (e.g., X-vivo, Lonza) or buffer (e.g., 1×PBS). 4) 1 μl of an adequate dilution (according to the manufacturer recommendations) of caspase-3/7 detection reagent (e.g., Thermo Fisher #C10427) is added. 5) Wells are incubated for 30 minutes in the dark at 37° C. or room temperature (according to the manufacturer recommendations). 6) Cells are analyzed by flow cytometry.

According to certain embodiments, the one or more amino acid substitutions are at positions corresponding to positions in the full length amino acid sequence of Fas (SEQ ID NO: 1) selected from the group consisting of V245, R250, K251, V254, E256, K258, I259, D260, E261, K263, E272, W281, Y291, K296 and L298. Preferably, the at least one amino acid substitution is a non-conservative substitution. More preferably, the at least one amino acid substitution is a non-conservative substitution affecting at least position K296.

According to particular embodiments, the one or more amino acid substitutions are at positions corresponding to positions in the full length amino acid sequence of Fas (SEQ ID NO: 1) selected from the group consisting of R250, V254, E256, D260, E261, K263, Y291 and K296. Preferably, the at least one amino acid substitution is a non-conservative substitution.

According to certain embodiments, the one or more amino acid substitutions are at positions corresponding to positions in the full length amino acid sequence of DR4 (SEQ ID NO: 2) selected from the group consisting of W380, R385, Q286, L289, K391, E293, I294, D295, V296, R298, D406, W415, 1425, D430, and L432. Preferably, the at least one amino acid substitution is a non-conservative substitution.

According to particular embodiments, the one or more amino acid substitutions are at positions corresponding to positions in the full length amino acid sequence of DR4 (SEQ ID NO: 2) selected from the group consisting of W380, Q286, K391, E293, D295, R298, D406 and L432. Preferably, the at least one amino acid substitution is a non-conservative substitution.

According to certain embodiments, the one or more amino acid substitutions are at positions corresponding to positions in the full length amino acid sequence of DR5 (SEQ ID NO: 3) selected from the group consisting of W354, R359, K360, L363, D365, E367, I368, K369, V370, K372, D380, W389, V399, D404 and L406. Preferably, the at least one amino acid substitution is a non-conservative substitution.

According to particular embodiments, the one or more amino acid substitutions are at positions corresponding to positions in the full length amino acid sequence of DR5 (SEQ ID NO: 3) selected from the group consisting of W354, K360, D365, E367, K369, K372, D380 and L406. Preferably, the at least one amino acid substitution is a non-conservative substitution.

According to certain embodiments, the one or more substitutions are at positions corresponding to positions in the full length amino acid sequence of TNFR1 (SEQ ID NO: 4) selected from the group consisting of W371, R376, R377, L380, D382, E384, I385, D386, R387, E389, E398, W407, L418, R423 and L425. Preferably, the at least one amino acid substitution is a non-conservative substitution.

According to certain embodiments, the one or more amino acid substitutions are at positions corresponding to positions in the full length amino acid sequence of DR3 (SEQ ID NO: 5) selected from the group consisting of W347, R352, T353, L356, E358, E360, I361, E362, E365, D373, W382, L390 and L397. Preferably, the at least one amino acid substitution is a non-conservative substitution.

According to certain embodiments, the death domain comprises, or consists of, an amino acid sequence having at least 80%, such as at least 85%, sequence identity with SEQ ID NO: 6, the amino acid sequence comprising at least one amino acid substitution at a position which corresponds to a position in SEQ ID NO: 6 selected from the group consisting of V16, R21, K22, V25, E27, K29, I30, D31, E32, K34, E43, W52, Y62, K67 and L69. Preferably, the at least one amino acid substitution is a non-conservative substitution.

According to certain embodiments, the death domain comprises, or consists of, an amino acid sequence having at least 85%, such as at least 90%, sequence identity with SEQ ID NO: 6, the amino acid sequence comprising at least one amino acid substitution at a position which corresponds to a position in SEQ ID NO: 6 selected from the group consisting of V16, R21, K22, V25, E27, K29, I30, D31, E32, K34, E43, W52, Y62, K67 and L69. Preferably, the at least one amino acid substitution is a non-conservative substitution.

According to certain embodiments, the death domain comprises, or consists of, an amino acid sequence having at least 90%, such as at least 95%, sequence identity with SEQ ID NO: 6, the amino acid sequence comprising at least one amino acid substitution at a position which corresponds to a position in SEQ ID NO: 6 selected from the group consisting of V16, R21, K22, V25, E27, K29, I30, D31, E32, K34, E43, W52, Y62, K67 and L69. Preferably, the at least one amino acid substitution is a non-conservative substitution.

According to certain embodiments, the death domain comprises, or consists of, an amino acid sequence which differs from the amino acid sequence of SEQ ID NO: 6 in that one or more amino acid residues at positions selected from the group consisting of V16, R21, K22, V25, E27, K29, 130, D31, E32, K34, E43, W52, Y62, K67 and L69 are substituted. Preferably, the one or more amino acid substitutions are non-conservative substitutions.

According to certain embodiments, the death domain comprises, or consists of, an amino acid sequence having at least 80%, such as at least 85%, sequence identity with SEQ ID NO: 6, the amino acid sequence comprising at least one amino acid substitution at a position which corresponds to a position in SEQ ID NO: 6 selected from the group consisting of R21, V25, E27, D31, E32, K34, Y62 and K67. Preferably, the at least one amino acid substitution is a non-conservative substitution.

According to certain embodiments, the death domain comprises, or consists of, an amino acid sequence having at least 85%, such as at least 90%, sequence identity with SEQ ID NO: 6, the amino acid sequence comprising at least one amino acid substitution at a position which corresponds to a position in SEQ ID NO: 6 selected from the group consisting of R21, V25, E27, D31, E32, K34, Y62 and K67. Preferably, the at least one amino acid substitution is a non-conservative substitution. According to particular embodiments, the at least one amino acid substitution is selected from the group consisting of R21A, V25N, E27A, D31A, E32A, K34A, Y62D and K67A. According to particular embodiments, the at least one amino acid substitution includes K67A.

According to certain embodiments, the death domain comprises, or consists of, an amino acid sequence having at least 90%, such as at least 95%, sequence identity with SEQ ID NO: 6, the amino acid sequence comprising at least one amino acid substitution at a position which corresponds to a position in SEQ ID NO: 6 selected from the group consisting of R21, V25, E27, D31, E32, K34, Y62 and K67. Preferably, the at least one amino acid substitution is a non-conservative substitution. According to particular embodiments, the at least one amino acid substitution is selected from the group consisting of R21A, V25N, E27A, D31A, E32A, K34A, Y62D and K67A. According to particular embodiments, the at least one amino acid substitution includes K67A.

According to certain embodiments, the death domain comprises, or consists of, an amino acid sequence having at least 80%, such as at least 85%, sequence identity with SEQ ID NO: 6, the amino acid sequence comprising a R to A substitution at position 21.

According to certain embodiments, the death domain comprises, or consists of, an amino acid sequence having at least 90%, such as at least 95%, sequence identity with SEQ ID NO: 6, the amino acid sequence comprising a R to A substitution at position 21.

According to certain embodiments, the death domain comprises, or consists of, an amino acid sequence having at least 80%, such as at least 85%, sequence identity with SEQ ID NO: 6, the amino acid sequence comprising a V to N substitution at position 25.

According to certain embodiments, the death domain comprises, or consists of, an amino acid sequence having at least 90%, such as at least 95%, sequence identity with SEQ ID NO: 6, the amino acid sequence comprising a V to N substitution at position 25.

According to certain embodiments, the death domain comprises, or consists of, an amino acid sequence having at least 80%, such as at least 85%, sequence identity with SEQ ID NO: 6, the amino acid sequence comprising an E to A substitution at position 27.

According to certain embodiments, the death domain comprises, or consists of, an amino acid sequence having at least 90%, such as at least 95%, sequence identity with SEQ ID NO: 6, the amino acid sequence comprising an E to A substitution at position 27.

According to certain embodiments, the death domain comprises, or consists of, an amino acid sequence having at least 80%, such as at least 85%, sequence identity with SEQ ID NO: 6, the amino acid sequence comprising a D to A substitution at position 31.

According to certain embodiments, the death domain comprises, or consists of, an amino acid sequence having at least 90%, such as at least 95%, sequence identity with SEQ ID NO: 6, the amino acid sequence comprising a D to A substitution at position 31.

According to certain embodiments, the death domain comprises, or consists of, an amino acid sequence having at least 80%, such as at least 85%, sequence identity with SEQ ID NO: 6, the amino acid sequence comprising an E to A substitution at position 32.

According to certain embodiments, the death domain comprises, or consists of, an amino acid sequence having at least 90%, such as at least 95%, sequence identity with SEQ ID NO: 6, the amino acid sequence comprising an E to A substitution at position 32.

According to certain embodiments, the death domain comprises, or consists of, an amino acid sequence having at least 80%, such as at least 85%, sequence identity with SEQ ID NO: 6, the amino acid sequence comprising a K to A substitution at position 34.

According to certain embodiments, the death domain comprises, or consists of, an amino acid sequence having at least 90%, such as at least 95%, sequence identity with SEQ ID NO: 6, the amino acid sequence comprising a K to A substitution at position 34.

According to certain embodiments, the death domain comprises, or consists of, an amino acid sequence having at least 80%, such as at least 85%, at least 90% or at least 95%, sequence identity with SEQ ID NO: 6, the amino acid sequence comprising a Y to D substitution at position 62.

According to certain embodiments, the death domain comprises, or consists of, an amino acid sequence having at least 90%, such as at least 95%, sequence identity with SEQ ID NO: 6, the amino acid sequence comprising a Y to D substitution at position 62.

According to certain embodiments, the death domain comprises, or consists of, an amino acid sequence having at least 80%, such as at least 85%, sequence identity with SEQ ID NO: 6, the amino acid sequence comprising a K to A substitution at position 67.

According to certain embodiments, the death domain comprises, or consists of, an amino acid sequence having at least 90%, such as at least 95%, sequence identity with SEQ ID NO: 6, the amino acid sequence comprising a K to A substitution at position 67.

According to certain embodiments, the death domain comprises, or consists of, an amino acid sequence which differs from the amino acid sequence of SEQ ID NO: 6 in that one or more amino acid residues at positions selected from the group consisting of R21, V25, E27, D31, E32, K34, Y62 and K67 are substituted. Preferably, the one or more amino acid substitutions are non-conservative substitutions. According to particular embodiments, the one or more substitutions are selected from the group consisting of R21A, V25N, E27A, D31A, E32A, K34A, Y62D and K67A.

According to certain embodiments, the death domain comprises, or consists of, an amino acid sequence having at least 80%, such as 85%, sequence identity with SEQ ID NO: 7, the amino acid sequence comprising at least one amino acid substitution at a position which corresponds to a position in SEQ ID NO: 7 selected from the group consisting of: W16, R21, Q22, L25, K27, E29, I30, D31, V32, R34, D42, W51, I61, D66 and L68. Preferably, the at least one amino acid substitution is a non-conservative substitution.

According to certain embodiments, the death domain comprises, or consists of, an amino acid sequence having at least 90%, such as 95%, sequence identity with SEQ ID NO: 7, the amino acid sequence comprising at least one amino acid substitution at a position which corresponds to a position in SEQ ID NO: 7 selected from the group consisting of: W16, R21, Q22, L25, K27, E29, I30, D31, V32, R34, D42, W51, I61, D66 and L68. Preferably, the at least one amino acid substitution is a non-conservative substitution.

According to certain embodiments, the death domain comprises, or consists of, an amino acid sequence which differs from the amino acid sequence of SEQ ID NO: 7 in that one or more amino acid residues at positions selected from the group consisting of W16, R21, Q22, L25, K27, E29, I30, D31, V32, R34, D42, W51, I61, D66 and L68 are substituted. Preferably, the one or more amino acid substitutions are non-conservative substitutions.

According to certain embodiments, the death domain comprises, or consists of, an amino acid sequence having at least 80%, such as at least 85%, sequence identity with SEQ ID NO: 7, the amino acid sequence comprising at least one amino acid substitution at a position which corresponds to a position in SEQ ID NO: 7 selected from the group consisting of: W16, Q22, K27, E29, D31, R34, D42 and L68. Preferably, the at least one amino acid substitution is a non-conservative substitution. According to particular embodiments, the at least one amino acid substitution is selected from the group consisting of: W16A, Q22A, K27A, E29A, D31A, R34A, D42A and L68A.

According to certain embodiments, the death domain comprises, or consists of, an amino acid sequence having at least 90%, such as at least 95%, sequence identity with SEQ ID NO: 7, the amino acid sequence comprising at least one amino acid substitution at a position which corresponds to a position in SEQ ID NO: 7 selected from the group consisting of: W16, Q22, K27, E29, D31, R34, D42 and L68. Preferably, the at least one amino acid substitution is a non-conservative substitution. According to particular embodiments, the at least one amino acid substitution is selected from the group consisting of: W16A, Q22A, K27A, E29A, D31A, R34A, D42A and L68A.

According to certain embodiments, the death domain comprises, or consists of, an amino acid sequence which differs from the amino acid sequence of SEQ ID NO: 7 in that one or more amino acid residues at positions selected from the group consisting of W16, Q22, K27, E29, D31, R34, D42 and L68 are substituted. Preferably, the one or more amino acid substitutions are non-conservative substitutions. According to particular embodiments, the one or more substitutions are selected from the group consisting of W16A, Q22A, K27A, E29A, D31A, R34A, D42A and L68A.

According to certain embodiments, the death domain comprises, or consists of, an amino acid sequence having at least 80%, such as at least 85%, sequence identity with SEQ ID NO: 8, the amino acid sequence comprising at least one amino acid substitution at a position which corresponds to a position in SEQ ID NO: 8 selected from the group consisting of: W16, R21, K22, L25, D27, E29, I30, K31, V32, K34, D42, W51, V61, D66 and L68. Preferably, the at least one amino acid substitution is a non-conservative substitution.

According to certain embodiments, the death domain comprises, or consists of, an amino acid sequence having at least 90%, such as at least 95%, sequence identity with SEQ ID NO: 8, the amino acid sequence comprising at least one amino acid substitution at a position which corresponds to a position in SEQ ID NO: 8 selected from the group consisting of: W16, R21, K22, L25, D27, E29, I30, K31, V32, K34, D42, W51, V61, D66 and L68. Preferably, the at least one amino acid substitution is a non-conservative substitution.

According to certain embodiments, the death domain comprises, or consists of, an amino acid sequence which differs from the amino acid sequence of SEQ ID NO: 8 in that one or more amino acid residues at positions selected from the group consisting of W16, R21, K22, L25, D27, E29, I30, K31, V32, K34, D42, W51, V61, D66 and L68 are substituted. Preferably, the one or more amino acid substitutions are non-conservative substitutions.

According to certain embodiments, the death domain comprises, or consists of, an amino acid sequence having at least 80%, such as at least 85%, sequence identity with SEQ ID NO: 8, the amino acid sequence comprising at least one amino acid substitution at a position which corresponds to a position in SEQ ID NO: 8 selected from the group consisting of: W16, K22, D27, E29, K31, K34, D42 and L68. Preferably, the at least one amino acid substitution is a non-conservative substitution. According to particular embodiments, the at least one amino acid substitution is selected from the group consisting of: W16A, K22A, D27A, E29A, K31A, K34A, D42A and L68A.

According to certain embodiments, the death domain comprises, or consists of, an amino acid sequence having at least 90%, such as at least 95%, sequence identity with SEQ ID NO: 8, the amino acid sequence comprising at least one amino acid substitution at a position which corresponds to a position in SEQ ID NO: 8 selected from the group consisting of: W16, K22, D27, E29, K31, K34, D42 and L68. Preferably, the at least one amino acid substitution is a non-conservative substitution. According to particular embodiments, the at least one amino acid substitution is selected from the group consisting of: W16A, K22A, D27A, E29A, K31A, K34A, D42A and L68A.

According to certain embodiments, the death domain comprises, or consists of, an amino acid sequence which differs from the amino acid sequence of SEQ ID NO: 8 in that one or more amino acid residues at positions selected from the group consisting of W16, K22, D27, E29, K31, K34, D42 and L68 are substituted. Preferably, the one or more amino acid substitutions are non-conservative substitutions. According to particular embodiments, the one or more substitutions are selected from the group consisting of W16A, K22A, D27A, E29A, K31A, K34A, D42A and L68A.

According to certain embodiments, the death domain comprises, or consists of, an amino acid sequence having at least 80%, such as at least 85%, sequence identity with SEQ ID NO: 9, the amino acid sequence comprising at least one amino acid substitution at a position which corresponds to a position in SEQ ID NO: 9 selected from the group consisting of W16, R21, R22, L25, D27, E29, I30, D31, R32, E34, E43, W52, L63, R68 and L70. Preferably, the at least one amino acid substitution is a non-conservative substitution.

According to certain embodiments, the death domain comprises, or consists of, an amino acid sequence having at least 90%, such as at least 95%, sequence identity with SEQ ID NO: 9, the amino acid sequence comprising at least one amino acid substitution at a position which corresponds to a position in SEQ ID NO: 9 selected from the group consisting of W16, R21, R22, L25, D27, E29, I30, D31, R32, E34, E43, W52, L63, R68 and L70. Preferably, the at least one amino acid substitution is a non-conservative substitution.

According to certain embodiments, the death domain comprises, or consists of, an amino acid sequence which differs from the amino acid sequence of SEQ ID NO: 9 in that one or more amino acid residues at positions selected from the group consisting of W16, R21, R22, L25, D27, E29, I30, D31, R32, E34, E43, W52, L63, R68 and L70 are substituted. Preferably, the one or more amino acid substitutions are non-conservative substitutions.

According to certain embodiments, the death domain comprises, or consists of, an amino acid sequence having at least 80%, such as at least 85%, sequence identity with SEQ ID NO: 9, the amino acid sequence comprising at least one amino acid substitution at a position which corresponds to a position in SEQ ID NO: 9 selected from the group consisting of W16, R22, D27, E29, D31, E34, E43 and L70. Preferably, the at least one amino acid substitution is a non-conservative substitution. According to particular embodiments, the at least one amino acid substitution is selected from the group consisting of W16A, R22A, D27A, E29A, D31A, E34A, E43A and L70A.

According to certain embodiments, the death domain comprises, or consists of, an amino acid sequence having at least 90%, such as at least 95%, sequence identity with SEQ ID NO: 9, the amino acid sequence comprising at least one amino acid substitution at a position which corresponds to a position in SEQ ID NO: 9 selected from the group consisting of W16, R22, D27, E29, D31, E34, E43 and L70. Preferably, the at least one amino acid substitution is a non-conservative substitution. According to particular embodiments, the at least one amino acid substitution is selected from the group consisting of W16A, R22A, D27A, E29A, D31A, E34A, E43A and L70A.

According to certain embodiments, the death domain comprises, or consists of, an amino acid sequence which differs from the amino acid sequence of SEQ ID NO: 9 in that one or more amino acid residues at positions selected from the group consisting of W16, R22, D27, E29, D31, E34, E43 and L70 are substituted. Preferably, the one or more amino acid substitutions are non-conservative substitutions. According to particular embodiments, the one or more substitutions are selected from the group consisting of W16A, R22A, D27A, E29A, D31A, E34A, E43A and L70A.

According to certain embodiments, the death domain comprises, or consists of, an amino acid sequence having at least 80%, such as at least 85%, sequence identity with SEQ ID NO: 10, the amino acid sequence comprising at least one amino acid substitution at a position which corresponds to a position in SEQ ID NO: 10 selected from the group consisting of W16, R21, T22, L25, E27, E29, I30, E31, E34, D42, W51, L59 and L66. Preferably, the at least one amino acid substitution is a non-conservative substitution.

According to certain embodiments, the death domain comprises, or consists of, an amino acid sequence having at least 90%, such as at least 95%, sequence identity with SEQ ID NO: 10, the amino acid sequence comprising at least one amino acid substitution at a position which corresponds to a position in SEQ ID NO: 10 selected from the group consisting of W16, R21, T22, L25, E27, E29, I30, E31, E34, D42, W51, L59 and L66. Preferably, the at least one amino acid substitution is a non-conservative substitution.

According to certain embodiments, the death domain comprises, or consists of, an amino acid sequence which differs from the amino acid sequence of SEQ ID NO: 10 in that one or more amino acid residues at positions selected from the group consisting of W16, R21, T22, L25, E27, E29, I30, E31, E34, D42, W51, L59 and L66 are substituted. Preferably, the one amino acid substitutions are non-conservative substitution.

According to certain embodiments, the death domain comprises, or consists of, an amino acid sequence having at least 80%, such as at least 85%, sequence identity with SEQ ID NO: 10, the amino acid sequence comprising at least one amino acid substitution at a position which corresponds to a position in SEQ ID NO: 10 selected from the group consisting of W16, T22, E27, E29, E31, E34, D42 and L66. Preferably, the at least one amino acid substitution is a non-conservative substitution. According to particular embodiments, the at least one amino acid substitution is selected from the group consisting of W16A, T22A, E27A, E29A, E31A, E34A, D42A and L66A.

According to certain embodiments, the death domain comprises, or consists of, an amino acid sequence having at least 90%, such as at least 95%, sequence identity with SEQ ID NO: 10, the amino acid sequence comprising at least one amino acid substitution at a position which corresponds to a position in SEQ ID NO: 10 selected from the group consisting of W16, T22, E27, E29, E31, E34, D42 and L66. Preferably, the at least one amino acid substitution is a non-conservative substitution. According to particular embodiments, the at least one amino acid substitution is selected from the group consisting of W16A, T22A, E27A, E29A, E31A, E34A, D42A and L66A.

According to certain embodiments, the death domain comprises, or consists of, an amino acid sequence which differs from the amino acid sequence of SEQ ID NO: 10 in that one or more amino acid residues at positions selected from the group consisting of W16, T22, E27, E29, E31, E34, D42 and L66 are substituted. Preferably, the one or more amino acid substitutions are non-conservative substitutions. According to particular embodiments, the one or more substitutions are selected from the group consisting of W16A, T22A, E27A, E29A, E31A, E34A, D42A and L66A.

According to certain embodiments, the endodomain comprises, or consists of, an amino acid sequence having at least 80%, such as at least 85%, sequence identity with SEQ ID NO: 11, the amino acid sequence comprising at least one amino acid substitution at a position which corresponds to a position in SEQ ID NO: 11 selected from the group consisting of V55, R60, K61, V64, E66, K68, I69, D70, E71, K73, E82, W91, Y101, K106 and L108. Preferably, the at least one amino acid substitution is a non-conservative substitution.

According to certain embodiments, the endodomain comprises, or consists of, an amino acid sequence having at least 85%, such as at least 90%, sequence identity with SEQ ID NO: 11, the amino acid sequence comprising at least one amino acid substitution at a position which corresponds to a position in SEQ ID NO: 11 selected from the group consisting of V55, R60, K61, V64, E66, K68, I69, D70, E71, K73, E82, W91, Y101, K106 and L108. Preferably, the at least one amino acid substitution is a non-conservative substitution.

According to certain embodiments, the endodomain comprises, or consists of, an amino acid sequence which differs from the amino acid sequence of SEQ ID NO: 11 in that one or more amino acid residues at positions selected from the group consisting of V55, R60, K61, V64, E66, K68, I69, D70, E71, K73, E82, W91, Y101, K106 and L108 are substituted. Preferably, the one or more amino acid substitutions are non-conservative substitutions.

According to certain embodiments, the endodomain comprises, or consists of, an amino acid sequence having at least 80%, such as at least 85%, sequence identity with SEQ ID NO: 11, the amino acid sequence comprising at least one amino acid substitution at a position which corresponds to a position in SEQ ID NO: 11 selected from the group consisting of R60, V64, E66, D70, E71, K73, Y101 and K106. Preferably, the at least one amino acid substitution is a non-conservative substitution. According to particular embodiments, the at least one amino acid substitution is selected from the group consisting of R60A, V64N, E66A, D70A, E71A, K73A, Y101D and K106A. According to particular embodiments, the at least one amino acid substitution includes K106A.

According to certain embodiments, the endodomain comprises, or consists of, an amino acid sequence having at least 90%, such as at least 95%, sequence identity with SEQ ID NO: 11, the amino acid sequence comprising at least one amino acid substitution at a position which corresponds to a position in SEQ ID NO: 11 selected from the group consisting of R60, V64, E66, D70, E71, K73, Y101 and K106. Preferably, the at least one amino acid substitution is a non-conservative substitution. According to particular embodiments, the at least one amino acid substitution is selected from the group consisting of R60A, V64N, E66A, D70A, E71A, K73A, Y101D and K106A. According to particular embodiments, the at least one amino acid substitution includes K106A.

According to certain embodiments, the endodomain comprises, or consists of, an amino acid sequence having at least 80%, such as at least 85%, sequence identity with SEQ ID NO: 11, the amino acid sequence comprising a R to A substitution at position 60.

According to certain embodiments, the endodomain comprises, or consists of, an amino acid sequence having at least 90%, such as at least 95%, sequence identity with SEQ ID NO: 11, the amino acid sequence comprising a R to A substitution at position 60.

According to certain embodiments, the endodomain comprises, or consists of, an amino acid sequence having at least 80%, such as at least 85%, sequence identity with SEQ ID NO: 11, the amino acid sequence comprising a V to N substitution at position 64.

According to certain embodiments, the endodomain comprises, or consists of, an amino acid sequence having at least 90%, such as at least 95%, sequence identity with SEQ ID NO: 11, the amino acid sequence comprising a V to N substitution at position 64.

According to certain embodiments, the endodomain comprises, or consists of, an amino acid sequence having at least 80%, such as at least 85%, sequence identity with SEQ ID NO: 11, the amino acid sequence comprising an E to A substitution at position 66.

According to certain embodiments, the endodomain comprises, or consists of, an amino acid sequence having at least 90%, such as at least 95%, sequence identity with SEQ ID NO: 11, the amino acid sequence comprising an E to A substitution at position 66.

According to certain embodiments, the endodomain comprises, or consists of, an amino acid sequence having at least 80%, such as at least 85%, sequence identity with SEQ ID NO: 11, the amino acid sequence comprising a D to A substitution at position 70.

According to certain embodiments, the endodomain comprises, or consists of, an amino acid sequence having at least 90%, such as at least 95%, sequence identity with SEQ ID NO: 11, the amino acid sequence comprising a D to A substitution at position 70.

According to certain embodiments, the endodomain comprises, or consists of, an amino acid sequence having at least 80%, such as at least 85%, sequence identity with SEQ ID NO: 11, the amino acid sequence comprising an E to A substitution at position 71.

According to certain embodiments, the endodomain comprises, or consists of, an amino acid sequence having at least 90%, such as at least 95%, sequence identity with SEQ ID NO: 11, the amino acid sequence comprising an E to A substitution at position 71.

According to certain embodiments, the endodomain comprises, or consists of, an amino acid sequence having at least 80%, such as at least 85%, sequence identity with SEQ ID NO: 11, the amino acid sequence comprising a K to A substitution at position 73.

According to certain embodiments, the endodomain comprises, or consists of, an amino acid sequence having at least 90%, such as at least 95%, sequence identity with SEQ ID NO: 11, the amino acid sequence comprising a K to A substitution at position 73.

According to certain embodiments, the endodomain comprises, or consists of, an amino acid sequence having at least 80%, such as at least 85%, sequence identity with SEQ ID NO: 11, the amino acid sequence comprising a Y to D substitution at position 101.

According to certain embodiments, the endodomain comprises, or consists of, an amino acid sequence having at least 90%, such as at least 95%, sequence identity with SEQ ID NO: 11, the amino acid sequence comprising a Y to D substitution at position 101.

According to certain embodiments, the endodomain comprises, or consists of, an amino acid sequence having at least 80%, such as at least 85%, sequence identity with SEQ ID NO: 11, the amino acid sequence comprising a K to A substitution at position 106.

According to certain embodiments, the endodomain comprises, or consists of, an amino acid sequence having at least 90%, such as at least 95%, sequence identity with SEQ ID NO: 11, the amino acid sequence comprising a K to A substitution at position 106.

According to certain embodiments, the endodomain comprises, or consists of, an amino acid sequence which differs from the amino acid sequence of SEQ ID NO: 11 in that one or more amino acid residues at positions selected from the group consisting of R60, V64, E66, D70, E71, K73, Y101 and K106 are substituted. Preferably, the one or more amino acid substitutions are non-conservative substitutions. According to particular embodiments, the one or more substitutions are selected from the group consisting of R60A, V64N, E66A, D70A, E71A, K73A, Y101D and K106A. According to particular embodiments, the amino acid sequence includes the substitution K67A.

According to certain embodiments, the endodomain comprises, or consists of, an amino acid sequence having at least 80%, such as at least 85%, sequence identity with SEQ ID NO: 12, the amino acid sequence comprising at least one amino acid substitution at a position which corresponds to a position in SEQ ID NO: 12 selected from the group consisting of: W118, R123, Q124, L127, K129, E131, I132, D133, V134, R136, D144, W153, I163, D168 and L170. Preferably, the at least one amino acid substitution is a non-conservative substitution.

According to certain embodiments, the endodomain comprises, or consists of, an amino acid sequence having at least 90%, such as at least 95%, sequence identity with SEQ ID NO: 12, the amino acid sequence comprising at least one amino acid substitution at a position which corresponds to a position in SEQ ID NO: 12 selected from the group consisting of: W118, R123, Q124, L127, K129, E131, I132, D133, V134, R136, D144, W153, I163, D168 and L170. Preferably, the at least one amino acid substitution is a non-conservative substitution.

According to certain embodiments, the endodomain comprises, or consists of, an amino acid sequence which differs from the amino acid sequence of SEQ ID NO: 12 in that one or more amino acid residues at positions selected from the group consisting of W118, R123, Q124, L127, K129, E131, I132, D133, V134, R136, D144, W153, I163, D168 and L170 are substituted. Preferably, the one or more amino acid substitutions are non-conservative substitutions.

According to certain embodiments, the endodomain comprises, or consists of, an amino acid sequence having at least 80%, such as at least 85%, sequence identity with SEQ ID NO: 12, the amino acid sequence comprising at least one amino acid substitution at a position which corresponds to a position in SEQ ID NO: 12 selected from the group consisting of: W118, Q124, K129, E131, D133, R136, D144 and L170. Preferably, the at least one amino acid substitution is a non-conservative substitution. According to particular embodiments, the at least one amino acid substitution is selected from the group consisting of: W118A, Q124A, K129A, E131A, D133A, R136A, D144A and L170A.

According to certain embodiments, the endodomain comprises, or consists of, an amino acid sequence having at least 90%, such as at least 95%, sequence identity with SEQ ID NO: 12, the amino acid sequence comprising at least one amino acid substitution at a position which corresponds to a position in SEQ ID NO: 12 selected from the group consisting of: W118, Q124, K129, E131, D133, R136, D144 and L170. Preferably, the at least one amino acid substitution is a non-conservative substitution. According to particular embodiments, the at least one amino acid substitution is selected from the group consisting of: W118A, Q124A, K129A, E131A, D133A, R136A, D144A and L170A.

According to certain embodiments, the endodomain comprises, or consists of, an amino acid sequence which differs from the amino acid sequence of SEQ ID NO: 12 in that one or more amino acid residues at positions selected from the group consisting of W118, Q124, K129, E131, D133, R136, D144 and L170 are substituted. Preferably, the one or more amino acid substitutions are non-conservative substitutions. According to particular embodiments, the one or more substitutions are selected from the group consisting of W118A, Q124A, K129A, E131A, D133A, R136A, D144A and L170A.

According to certain embodiments, the endodomain comprises, or consists of, an amino acid sequence having at least 80%, such as at least 85%, sequence identity with SEQ ID NO: 13, the amino acid sequence comprising at least one amino acid substitution at a position which corresponds to a position in SEQ ID NO: 13 selected from the group consisting of: W123, R128, K129, L132, D134, E136, I137, K138, V139, K141, D149, W158, V168, D173 and L175. Preferably, the at least one amino acid substitution is a non-conservative substitution.

According to certain embodiments, the endodomain comprises, or consists of, an amino acid sequence having at least 90%, such as at least 95%, sequence identity with SEQ ID NO: 13, the amino acid sequence comprising at least one amino acid substitution at a position which corresponds to a position in SEQ ID NO: 13 selected from the group consisting of: W123, R128, K129, L132, D134, E136, I137, K138, V139, K141, D149, W158, V168, D173 and L175. Preferably, the at least one amino acid substitution is a non-conservative substitution.

According to certain embodiments, the endodomain comprises, or consists of, an amino acid sequence which differs from the amino acid sequence of SEQ ID NO: 13 in that one or more amino acid residues at positions selected from the group consisting of W123, R128, K129, L132, D134, E136, I137, K138, V139, K141, D149, W158, V168, D173 and L175 are substituted. Preferably, the one or more amino acid substitutions are non-conservative substitution.

According to certain embodiments, the endodomain comprises, or consists of, an amino acid sequence having at least 80%, such as at least 85%, sequence identity with SEQ ID NO: 13, the amino acid sequence comprising at least one amino acid substitution at a position which corresponds to a position in SEQ ID NO: 13 selected from the group consisting of: W123, K129, D134, E136, K138, K141, D149 and L175. Preferably, the at least one amino acid substitution is a non-conservative substitution. According to particular embodiments, the at least one amino acid substitution is selected from the group consisting of: W123A, K129A, D134A, E136A, K138A, K141A, D149A and L175A.

According to certain embodiments, the endodomain comprises, or consists of, an amino acid sequence having at least 80%, such as at least 85%, sequence identity with SEQ ID NO: 13, the amino acid sequence comprising at least one amino acid substitution at a position which corresponds to a position in SEQ ID NO: 13 selected from the group consisting of: W123, K129, D134, E136, K138, K141, D149 and L175. Preferably, the at least one amino acid substitution is a non-conservative substitution. According to particular embodiments, the at least one amino acid substitution is selected from the group consisting of: W123A, K129A, D134A, E136A, K138A, K141A, D149A and L175A.

According to certain embodiments, the endodomain comprises, or consists of, an amino acid sequence which differs from the amino acid sequence of SEQ ID NO: 13 in that one or more amino acid residues at positions selected from the group consisting of W123, K129, D134, E136, K138, K141, D149 and L175 are substituted. Preferably, the one or more substitutions are non-conservative substitutions. According to particular embodiments, the one or more substitutions are selected from the group consisting of W123A, K129A, D134A, E136A, K138A, K141A, D149A and L175A.

According to certain embodiments, the endodomain comprises, or consists of, an amino acid sequence having at least 80%, such as at least 85%, sequence identity with SEQ ID NO: 14, the amino acid sequence comprising at least one amino acid substitution at a position which corresponds to a position in SEQ ID NO: 14 selected from the group consisting of W137, R142, R143, L146, D148, E150, I151, D152, R153, E155, E43, W173, L184, R189 and L191. Preferably, the at least one amino acid substitution is a non-conservative substitution.

According to certain embodiments, the endodomain comprises, or consists of, an amino acid sequence having at least 90%, such as at least 95%, sequence identity with SEQ ID NO: 14, the amino acid sequence comprising at least one amino acid substitution at a position which corresponds to a position in SEQ ID NO: 14 selected from the group consisting of W137, R142, R143, L146, D148, E150, I151, D152, R153, E155, E43, W173, L184, R189 and L191. Preferably, the at least one amino acid substitution is a non-conservative substitution.

According to certain embodiments, the endodomain comprises, or consists of, an amino acid sequence which differs from the amino acid sequence of SEQ ID NO: 14 in that one or more amino acid residues at positions selected from the group consisting of W137, R142, R143, L146, D148, E150, I151, D152, R153, E155, E164, W173, L184, R189 and L191 are substituted. Preferably, the one or more amino acid substitutions are non-conservative substitutions.

According to certain embodiments, the endodomain comprises, or consists of, an amino acid sequence having at least 80%, such as at least 85%, sequence identity with SEQ ID NO: 14, the amino acid sequence comprising at least one amino acid substitution at a position which corresponds to a position in SEQ ID NO: 14 selected from the group consisting of W137, R143, D148, E150, D152, E155, E164 and L191. Preferably, the at least one amino acid substitution is a non-conservative substitution. According to particular embodiments, the at least one amino acid substitution is selected from the group consisting of W137A, R143A, D148A, E150A, D152A, E155A, E164A and L191A.

According to certain embodiments, the endodomain comprises, or consists of, an amino acid sequence having at least 85%, such as at least 90%, sequence identity with SEQ ID NO: 14, the amino acid sequence comprising at least one amino acid substitution at a position which corresponds to a position in SEQ ID NO: 14 selected from the group consisting of W137, R143, D148, E150, D152, E155, E164 and L191. Preferably, the at least one amino acid substitution is a non-conservative substitution. According to particular embodiments, the at least one amino acid substitution is selected from the group consisting of W137A, R143A, D148A, E150A, D152A, E155A, E164A and L191A.

According to certain embodiments, the endodomain comprises, or consists of, an amino acid sequence which differs from the amino acid sequence of SEQ ID NO: 14 in that one or more amino acid residues at positions selected from the group consisting of W137, R143, D148, E150, D152, E155, E164 and L191 are substituted. Preferably, the one or more amino acid substitutions are non-conservative substitutions.

According to particular embodiments, the one or more substitutions are selected from the group consisting of W137A, R143A, D148A, E150A, D152A, E155A, E164A and L191A.

According to certain embodiments, the endodomain comprises, or consists of, an amino acid sequence having at least 80%, such as at least 85%, sequence identity with SEQ ID NO: 15, the amino acid sequence comprising at least one amino acid substitution at a position which corresponds to a position in SEQ ID NO: 15 selected from the group consisting of W127, R132, T133, L136, E138, E140, I141, E142, E145, D153, W162, L170 and L177. Preferably, the at least one amino acid substitution is a non-conservative substitution.

According to certain embodiments, the endodomain comprises, or consists of, an amino acid sequence having at least 90%, such as at least 95%, sequence identity with SEQ ID NO: 15, the amino acid sequence comprising at least one amino acid substitution at a position which corresponds to a position in SEQ ID NO: 15 selected from the group consisting of W127, R132, T133, L136, E138, E140, I141, E142, E145, D153, W162, L170 and L177. Preferably, the at least one amino acid substitution is a non-conservative substitution.

According to certain embodiments, the endodomain comprises, or consists of, an amino acid sequence which differs from the amino acid sequence of SEQ ID NO: 15 in that one or more amino acid residues at positions selected from the group consisting of W127, R132, T133, L136, E138, E140, I141, E142, E145, D153, W162, L170 and L177 are substituted. Preferably, the one or more amino acid substitutions are non-conservative substitutions.

According to certain embodiments, the endodomain comprises, or consists of, an amino acid sequence having at least 80%, such as at least 85%, sequence identity with SEQ ID NO: 15, the amino acid sequence comprising at least one amino acid substitution at a position which corresponds to a position in SEQ ID NO: 15 selected from the group consisting of W127, T133, E138, E140, E142, E145, D153 and L177. Preferably, the at least one amino acid substitution is a non-conservative substitution. According to particular embodiments, the at least one amino acid substitution is selected from the group consisting of W127A, T133A, E138A, E140A, E142A, E145A, D153A and L177A.

According to certain embodiments, the endodomain comprises, or consists of, an amino acid sequence having at least 90%, such as at least 95%, sequence identity with SEQ ID NO: 15, the amino acid sequence comprising at least one amino acid substitution at a position which corresponds to a position in SEQ ID NO: 15 selected from the group consisting of W127, T133, E138, E140, E142, E145, D153 and L177. Preferably, the at least one amino acid substitution is a non-conservative substitution. According to particular embodiments, the at least one amino acid substitution is selected from the group consisting of W127A, T133A, E138A, E140A, E142A, E145A, D153A and L177A.

According to certain embodiments, the endodomain comprises, or consists of, an amino acid sequence which differs from the amino acid sequence of SEQ ID NO: 15 in that one or more amino acid residues at positions selected from the group consisting of W127, T133, E138, E140, E142, E145, D153 and L177 are substituted. Preferably, the one or more amino acid substitutions are non-conservative substitutions. According to particular embodiments, the one or more substitutions are selected from the group consisting of W127A, T133A, E138A, E140A, E142A, E145A, D153A and L177A.

A cell death inducing chimeric antigen receptor according to the present invention preferably comprises a hinge within the at least one ectodomain. The hinge is suitably located between the extracellular ligand-binding domain and the transmembrane domain.

The term “hinge” or “hinge region” used herein generally means any oligo- or polypeptide that functions to link the transmembrane domain to the extracellular ligand-binding domain. In particular, a hinge is used to provide more flexibility and accessibility for the extracellular ligand-binding domain. A hinge region may comprise up to 300 amino acids, preferably 10 to 100 amino acids and most preferably 25 to 50 amino acids. A hinge may be derived from all or part of naturally occurring molecules, such as from all or part of the extracellular region of Fas, DR4, DR5, TNFR1, DR3, CD8, CD4 or CD28, or from all or part of an antibody constant region. Alternatively the hinge may be a synthetic sequence that corresponds to a naturally occurring hinge sequence, or may be an entirely synthetic hinge sequence. Non-limiting examples of hinges which may be used in accordance to the invention include a part of human CD8 alpha chain, FcγRIIIα receptor or IgG1.

According to certain embodiments, the hinge is derived from the extracellular domain of a death receptor.

According to certain embodiments, the hinge is derived from the extracellular domain of a transmembrane receptor of the tumor necrosis factor (TNF) superfamily death receptor.

According to certain embodiments, the hinge is derived from the extracellular domain of Fas (CD95).

According to certain embodiments, the hinge comprises, or consists of, an amino acid sequence having at least 80%, such as at least 85%, sequence identity with any one of SEQ ID NOs: 16 to 18.

According to certain embodiments, the hinge comprises, or consists of, an amino acid sequence having at least 90%, such as at least 95%, sequence identity with any one of SEQ ID NOs: 16 to 18.

According to certain embodiments, the hinge comprises, or consists of, an amino acid sequence having at least 80%, such as at least 85%, sequence identity with SEQ ID NO: 16.

According to certain embodiments, the hinge comprises, or consists of, an amino acid sequence having at least 90%, such as at least 95%, sequence identity with SEQ ID NO: 16.

According to certain embodiments, the hinge comprises, or consists of, an amino acid sequence having at least 80%, such as at least 85%, sequence identity with SEQ ID NO: 17.

According to certain embodiments, the hinge comprises, or consists of, an amino acid sequence having at least 90%, such as at least 95%, sequence identity with SEQ ID NO: 17.

According to certain embodiments, the hinge comprises, or consists of, an amino acid sequence having at least 80%, such as at least 85%, sequence identity with SEQ ID NO: 18.

According to certain embodiments, the hinge comprises, or consists of, an amino acid sequence having at least 90%, such as at least 95%, sequence identity with SEQ ID NO: 18.

According to certain embodiments, the hinge is derived from the extracellular domain of DR4. According to particular embodiments, the hinge comprises, or consists of, an amino acid sequence having at least 80%, such as at least 85%, sequence identity with SEQ ID NO: 19.

According to particular embodiments, the hinge comprises, or consists of, an amino acid sequence having at least 90%, such as at least 95%, sequence identity with SEQ ID NO: 19.

According to certain embodiments, the hinge is derived from the extracellular domain of DR5. According to particular embodiments, the hinge comprises, or consists of, an amino acid sequence having at least 80%, such as at least 85%, sequence identity with SEQ ID NO: 20.

According to particular embodiments, the hinge comprises, or consists of, an amino acid sequence having at least 90%, such as at least 95%, sequence identity with SEQ ID NO: 20.

According to certain embodiments, the hinge is derived from the extracellular domain of DR3. According to particular embodiments, the hinge comprises, or consists of, an amino acid sequence having at least 80%, such as at least 85%, sequence identity with SEQ ID NO: 21.

According to particular embodiments, the hinge comprises, or consists of, an amino acid sequence having at least 90%, such as at least 95%, sequence identity with SEQ ID NO: 21.

According to certain embodiments, the hinge is selected from the group consisting of CD8a hinge, IgG1 hinge and FcγRIIIα hinge. According to particular embodiments, the hinge is selected from the group consisting of IgG1 hinge and FcγRIIIα hinge. According to more particular embodiments, the hinge is a IgG1 hinge.

According to certain embodiments, the hinge is a CD8a hinge. According to particular embodiments, the hinge comprises, or consists of, an amino acid sequence having at least 80%, such as at least 85%, sequence identity with SEQ ID NO: 22. According to particular embodiments, the hinge comprises, or consists of, an amino acid sequence having at least 90%, such as at least 95%, sequence identity with SEQ ID NO: 22.

According to certain embodiments, the hinge comprises, or consists of, an amino acid sequence having at least 80%, such as at least 85%, sequence identity with SEQ ID NO: 23. According to particular embodiments, the hinge comprises, or consists of, an amino acid sequence having at least 90%, such as at least 95%, sequence identity with SEQ ID NO: 23.

According to certain embodiments, the hinge is a FcγRIIIα hinge. According to particular embodiments, the hinge comprises, or consists of, an amino acid sequence having at least 80%, such as at least 85%, sequence identity with SEQ ID NO: 24. According to particular embodiments, the hinge comprises, or consists of, an amino acid sequence having at least 90%, such as at least 95%, sequence identity with SEQ ID NO: 24.

According to certain embodiments, a cell death inducing chimeric antigen receptor is provided wherein the hinge comprises one or more (such as two or more), such as 1 to 8 (e.g., 1, 2, 3, 4, 5, 6, 7 or 8), mutations in its amino acid sequence compared to the amino acid sequence of the wild type hinge from which it is derived (such as the hinge derived from Fas), which mutation(s) attenuate(s) the self-association of the cell death inducing chimeric antigen receptor.

According to certain embodiments, the hinge comprises one or more (such as two or more), such as 1 to 8 (e.g., 1, 2, 3, 4, 5, 6, 7 or 8), amino acid substitutions, preferably non-conservative amino acid substitutions, in its amino acid sequence compared to the amino acid sequence of the wild type hinge from which it is derived (such as the hinge derived from Fas), which amino acid substitution(s) attenuate(s) the self-association of the cell death inducing chimeric antigen receptor.

Generally, a mutation, such as an amino acid substitution, in the hinge is considered as being beneficial if it permits a target specific elimination in range with its wild type (unmutated) counterpart and if it improves the viability (less basal cell death) of an cell death inducing CAR positive cell population overtime in the absence of cell death inducing CAR target cells when compared to its wild type (unmutated) counterpart. In this respect, immune cells, such as primary T-cells, expressing the cell death inducing CAR are tested for their ability to be eliminated by target cells presenting the cell death inducing CAR target antigen. In addition, the viability (cell count) of the cell death inducing CAR positive population is followed over time (up to 20 days). Suitable methods for detecting apoptotic, necrotic, and dead cells have been described above in detail.

A cell death inducing chimeric antigen receptor according to the invention comprises at least one transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The at least one transmembrane domain can be derived either from a natural or from a synthetic source. The at least one transmembrane domain can be derived from any membrane-bound or transmembrane protein. As non-limiting examples, the at least one transmembrane domain can be a subunit of the T cell receptor such as α, β, γ or δ, polypeptide constituting CD3 complex, IL2 receptor p55 (α chain), p75 (β chain) or γ chain, a subunit chain of Fc receptors, in particular Fcγ receptor III or CD proteins. Alternatively, the at least one transmembrane domain can be synthetic and can comprise predominantly hydrophobic residues such as leucine and valine.

According to certain embodiments, the transmembrane domain is selected from the group consisting of the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI Ia, CD18), ICOS (CD278), 4-1BB (CD137), GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, LFA-1, ITGAM, CDI Ib, ITGAX, CDI Ic, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D or NKG2C.

According to certain embodiments, the at least one transmembrane domain is selected from the group consisting of CD95 (Fas) transmembrane domain, DR4 transmembrane domain, DR5 transmembrane domain, TNFR1 transmembrane domain, DR3 transmembrane domain, CD8 alpha transmembrane domain, 4-1BB transmembrane domain, DAP10 transmembrane domain and CD28 transmembrane domain.

According to particular embodiments, the at least one transmembrane domain is selected from the group consisting of human CD95 (Fas) transmembrane domain, human DR4 transmembrane domain, human DR5 transmembrane domain, human TNFR1 transmembrane domain, human DR3 transmembrane domain, human CD8 alpha transmembrane domain, human 4-1BB transmembrane domain, human DAP10 transmembrane domain and human CD28 transmembrane domain.

According to certain embodiments, the at least one transmembrane domain and the at least one endodomain of the cell death inducing CAR are derived from the same death receptor. For example, the at least one transmembrane domain and the at least one endodomain of the cell death inducing CAR may both be derived from CD95 (Fas), such as human CD95 (Fas).

According to certain embodiments, the at least one transmembrane domain is a CD95 (Fas) transmembrane domain. According to particular embodiments, the at least one transmembrane domain comprises, or consists of, an amino acid sequence having at least 80%, such as at least 85%, sequence identity with SEQ ID NO: 25. According to particular embodiments, the hinge comprises, or consists of, an amino acid sequence having at least 90%, such as at least 95%, sequence identity with SEQ ID NO: 25.

According to certain embodiments, the at least one transmembrane domain is a DR4 transmembrane domain. According to particular embodiments, the at least one transmembrane domain comprises, or consists of, an amino acid sequence having at least 80%, such as at least 85%, sequence identity with SEQ ID NO: 26. According to particular embodiments, the hinge comprises, or consists of, an amino acid sequence having at least 90%, such as at least 95%, sequence identity with SEQ ID NO: 26.

According to certain embodiments, the at least one transmembrane domain is a DR5 transmembrane domain. According to particular embodiments, the at least one transmembrane domain comprises, or consists of, an amino acid sequence having at least 80%, such as at least 85%, sequence identity with SEQ ID NO: 27. According to particular embodiments, the hinge comprises, or consists of, an amino acid sequence having at least 90%, such as at least 95%, sequence identity with SEQ ID NO: 27.

According to certain embodiments, the at least one transmembrane domain is a TNFR1 transmembrane domain. According to particular embodiments, the at least one transmembrane domain comprises, or consists of, an amino acid sequence having at least 80%, such as at least 85%, sequence identity with SEQ ID NO: 28. According to particular embodiments, the hinge comprises, or consists of, an amino acid sequence having at least 90%, such as at least 95%, sequence identity with SEQ ID NO: 28.

According to certain embodiments, the at least one transmembrane domain is a DR3 transmembrane domain. According to particular embodiments, the at least one transmembrane domain comprises, or consists of, an amino acid sequence having at least 80%, such as at least 85%, sequence identity with SEQ ID NO: 29. According to particular embodiments, the hinge comprises, or consists of, an amino acid sequence having at least 90%, such as at least 95%, sequence identity with SEQ ID NO: 29.

According to certain embodiments, the at least one transmembrane domain is a CD8 alpha transmembrane domain. According to particular embodiments, the at least one transmembrane domain comprises, or consists of, an amino acid sequence having at least 80%, such as at least 85%, sequence identity with SEQ ID NO: 30. According to particular embodiments, the hinge comprises, or consists of, an amino acid sequence having at least 90%, such as at least 95%, sequence identity with SEQ ID NO: 30.

According to certain embodiments, the at least one transmembrane domain is a 4-1BB transmembrane domain. According to particular embodiments, the at least one transmembrane domain comprises, or consists of, an amino acid sequence having at least 80%, such as at least 85%, sequence identity with SEQ ID NO: 31. According to particular embodiments, the hinge comprises, or consists of, an amino acid sequence having at least 90%, such as at least 95%, sequence identity with SEQ ID NO: 31.

According to certain embodiments, the at least one transmembrane domain is a DAP10 transmembrane domain. According to particular embodiments, the at least one transmembrane domain comprises, or consists of, an amino acid sequence having at least 80%, such as at least 85%, sequence identity with SEQ ID NO: 32. According to particular embodiments, the hinge comprises, or consists of, an amino acid sequence having at least 90%, such as at least 95%, sequence identity with SEQ ID NO: 32.

According to certain embodiments, the at least one transmembrane domain is a CD28 transmembrane domain. According to particular embodiments, the at least one transmembrane domain comprises, or consists of, an amino acid sequence having at least 80%, such as at least 85%, sequence identity with SEQ ID NO: 33. According to particular embodiments, the hinge comprises, or consists of, an amino acid sequence having at least 90%, such as at least 95%, sequence identity with SEQ ID NO: 33.

According to certain embodiments, the at least one transmembrane domain is selected from the group consisting of the transmembrane domains of the FcεRI α, β and γ chains. According to certain embodiments, the at least one transmembrane domain is selected from the group consisting of the transmembrane domains of the human FcεRI α, β and γ chains. According to particular embodiments, the at least one transmembrane domain comprises, or consists of, an amino acid sequence having at least 80%, such as at least 85%, sequence identity with SEQ ID NO: 34. According to particular embodiments, the hinge comprises, or consists of, an amino acid sequence having at least 90%, such as at least 95%, sequence identity with SEQ ID NO: 34. According to particular embodiments, the at least one transmembrane domain comprises, or consists of, an amino acid sequence having at least 80%, such as at least 85%, sequence identity with SEQ ID NO: 35. According to particular embodiments, the hinge comprises, or consists of, an amino acid sequence having at least 90%, such as at least 95%, sequence identity with SEQ ID NO: 35. According to particular embodiments, the at least one transmembrane domain comprises, or consists of, an amino acid sequence having at least 80%, such as at least 85%, sequence identity with SEQ ID NO: 36. According to particular embodiments, the hinge comprises, or consists of, an amino acid sequence having at least 90%, such as at least 95%, sequence identity with SEQ ID NO: 36.

As indicated above, the viability of immune cells expressing a cell death inducing chimeric antigen receptor may be maintained or even improved (less basal cell death) by attenuating the self-association of the receptor via the death domain and/or transmembrane domain. Generally, a mutation, such as an amino acid substitution, in the transmembrane domain is considered as being beneficial if it permits a target specific elimination in range with its wild type (unmutated) counterpart and if it improves the viability (less basal cell death) of an cell death inducing CAR positive cell population overtime in the absence of cell death inducing CAR target cells when compared to its wild type (unmutated) counterpart. In this respect, immune cells, such as primary T-cells, expressing the cell death inducing CAR are tested for their ability to be eliminated by target cells presenting the cell death inducing CAR target antigen. In addition, the viability (cell count) of the cell death inducing CAR positive population is followed over time (up to 20 days). Suitable methods for detecting apoptotic, necrotic, and dead cells have been described above in detail.

Therefore, according to certain embodiments, a cell death inducing chimeric antigen receptor is provided wherein the transmembrane domain comprises one or more (such as two or more), such as 1 to 8 (e.g., 1, 2, 3, 4, 5, 6, 7 or 8), mutations in its amino acid sequence compared to the amino acid sequence of the wild type transmembrane domain from which it is derived (such as the transmembrane domain derived from Fas), which mutation(s) attenuate(s) the self-association of the cell death inducing chimeric antigen receptor.

According to certain embodiments, the transmembrane domain comprises one or more (such as two or more), such as 1 to 8 (e.g., 1, 2, 3, 4, 5, 6, 7 or 8), amino acid substitutions, preferably non-conservative amino acid substitutions, in its amino acid sequence compared to the amino acid sequence of the wild type transmembrane domain from which it is derived (such as the transmembrane domain derived from Fas), which amino acid substitution(s) attenuate(s) the self-association of the cell death inducing chimeric antigen receptor.

According to certain embodiments, the one or more amino acid substitutions are at positions corresponding to positions in the full length amino acid sequence of Fas (SEQ ID NO: 1) selected from the group consisting of C178, L180, L180, P183, I184, P185, L186 and I187. Preferably, the at least one amino acid substitution is a non-conservative substitution. According to particular embodiments, the at least one amino acid substitution is selected from the group consisting of C178R, C178A, L180F, L180A, P183L, P183A, I184A, P185A, L186A and I187A.

According to certain embodiments, the transmembrane domain comprises, or consists of, an amino acid sequence having at least 80%, such as at least 85%, sequence identity with SEQ ID NO: 25, the amino acid sequence comprising at least one amino acid substitution at a position which corresponds to a position in SEQ ID NO: 25 selected from the group consisting of C5, L7, L7, P10, I11, P12, L13 and I14. Preferably, the at least one amino acid substitution is a non-conservative substitution. According to particular embodiments, the at least one amino acid substitution is selected from the group consisting of C5R, C5A, L7F, L7A, P10L, P10A, I11A, P12A, L13A and I14A.

According to certain embodiments, the transmembrane domain comprises, or consists of, an amino acid sequence having at least 85%, such as at least 90%, sequence identity with SEQ ID NO: 25, the amino acid sequence comprising at least one amino acid substitution at a position which corresponds to a position in SEQ ID NO: 25 selected from the group consisting of C5, L7, L7, P10, I11, P12, L13 and I14. Preferably, the at least one amino acid substitution is a non-conservative substitution. According to particular embodiments, the at least one amino acid substitution is selected from the group consisting of C5R, C5A, L7F, L7A, P10L, P10A, I11A, P12A, L13A and I14A.

According to certain embodiments, the transmembrane domain comprises, or consists of, an amino acid sequence having at least 90%, such as at least 95%, sequence identity with SEQ ID NO: 25, the amino acid sequence comprising at least one amino acid substitution at a position which corresponds to a position in SEQ ID NO: 25 selected from the group consisting of C5, L7, L7, P10, I11, P12, L13 and I14. Preferably, the at least one amino acid substitution is a non-conservative substitution. According to particular embodiments, the at least one amino acid substitution is selected from the group consisting of C5R, C5A, L7F, L7A, P10L, P10A, I11A, P12A, L13A and I14A.

According to certain embodiments, the transmembrane domain comprises, or consists of, an amino acid sequence which differs from the amino acid sequence of SEQ ID NO: 25 in that one or more amino acid residues at positions selected from the group consisting of C5, L7, L7, P10, I11, P12, L13 and I14 are substituted. Preferably, the one or more amino acid substitutions are non-conservative substitutions. According to particular embodiments, the at least one amino acid substitution is selected from the group consisting of C5R, C5A, L7F, L7A, P10L, P10A, I11A, P12A, L13A and I14A.

According to certain embodiments, the transmembrane domain comprises, or consists of, an amino acid sequence having at least 80%, such as at least 85%, sequence identity with SEQ ID NO: 25, the amino acid sequence comprising a C to R substitution at position 5.

According to certain embodiments, the transmembrane domain comprises, or consists of, an amino acid sequence having at least 90%, such as at least 95%, sequence identity with SEQ ID NO: 25, the amino acid sequence comprising a C to R substitution at position 5.

According to certain embodiments, the transmembrane domain comprises, or consists of, an amino acid sequence having at least 80%, such as at least 85%, sequence identity with SEQ ID NO: 25, the amino acid sequence comprising a C to A substitution at position 5.

According to certain embodiments, the transmembrane domain comprises, or consists of, an amino acid sequence having at least 90%, such as at least 95%, sequence identity with SEQ ID NO: 25, the amino acid sequence comprising a C to A substitution at position 5.

According to certain embodiments, the transmembrane domain comprises, or consists of, an amino acid sequence having at least 80%, such as at least 85%, sequence identity with SEQ ID NO: 25, the amino acid sequence comprising a L to F substitution at position 7.

According to certain embodiments, the transmembrane domain comprises, or consists of, an amino acid sequence having at least 90%, such as at least 95%, sequence identity with SEQ ID NO: 25, the amino acid sequence comprising a L to F substitution at position 7.

According to certain embodiments, the transmembrane domain comprises, or consists of, an amino acid sequence having at least 80%, such as at least 85%, sequence identity with SEQ ID NO: 25, the amino acid sequence comprising a L to A substitution at position 7.

According to certain embodiments, the transmembrane domain comprises, or consists of, an amino acid sequence having at least 90%, such as at least 95%, sequence identity with SEQ ID NO: 25, the amino acid sequence comprising a L to A substitution at position 7.

According to certain embodiments, the transmembrane domain comprises, or consists of, an amino acid sequence having at least 80%, such as at least 85%, sequence identity with SEQ ID NO: 25, the amino acid sequence comprising a P to L substitution at position 10.

According to certain embodiments, the transmembrane domain comprises, or consists of, an amino acid sequence having at least 90%, such as at least 95%, sequence identity with SEQ ID NO: 25, the amino acid sequence comprising a P to L substitution at position 10.

According to certain embodiments, the transmembrane domain comprises, or consists of, an amino acid sequence having at least 80%, such as at least 85%, sequence identity with SEQ ID NO: 25, the amino acid sequence comprising a P to A substitution at position 10.

According to certain embodiments, the transmembrane domain comprises, or consists of, an amino acid sequence having at least 90%, such as at least 95%, sequence identity with SEQ ID NO: 25, the amino acid sequence comprising a P to A substitution at position 10.

According to certain embodiments, the transmembrane domain comprises, or consists of, an amino acid sequence having at least 80%, such as at least 85%, sequence identity with SEQ ID NO: 25, the amino acid sequence comprising a I to A substitution at position 11.

According to certain embodiments, the transmembrane domain comprises, or consists of, an amino acid sequence having at least 90%, such as at least 95%, sequence identity with SEQ ID NO: 25, the amino acid sequence comprising a I to A substitution at position 11.

According to certain embodiments, the transmembrane domain comprises, or consists of, an amino acid sequence having at least 80%, such as at least 85%, sequence identity with SEQ ID NO: 25, the amino acid sequence comprising a P to A substitution at position 12.

According to certain embodiments, the transmembrane domain comprises, or consists of, an amino acid sequence having at least 90%, such as at least 95%, sequence identity with SEQ ID NO: 25, the amino acid sequence comprising a P to A substitution at position 12.

According to certain embodiments, the transmembrane domain comprises, or consists of, an amino acid sequence having at least 80%, such as at least 85%, sequence identity with SEQ ID NO: 25, the amino acid sequence comprising a L to A substitution at position 13.

According to certain embodiments, the transmembrane domain comprises, or consists of, an amino acid sequence having at least 90%, such as at least 95%, sequence identity with SEQ ID NO: 25, the amino acid sequence comprising a L to A substitution at position 13.

According to certain embodiments, the transmembrane domain comprises, or consists of, an amino acid sequence having at least 80%, such as at least 85%, sequence identity with SEQ ID NO: 25, the amino acid sequence comprising a I to A substitution at position 14.

According to certain embodiments, the transmembrane domain comprises, or consists of, an amino acid sequence having at least 90%, such as at least 95%, sequence identity with SEQ ID NO: 25, the amino acid sequence comprising a I to A substitution at position 14.

The “extracellular ligand-binding domain” comprised by the ectodomain of the cell death inducing chimeric antigen receptor may be any oligo- or polypeptide that is capable of binding a ligand, more specifically an antigen. However, it should be understood that the extracellular ligand-binding domain is not the ligand-binding domain naturally occurring in the wild type death receptor from which the death domain or endodomain including the death domain is derived. By way of example, if the death domain or endodomain including the death domain is derived from Fas (CD95), the extracellular ligand-binding domain is not a FasL-binding domain, i.e. it is not capable of binding FasL. Preferably, the extracellular ligand-binding domain will be capable of interacting with a cell surface molecule. For example, the extracellular ligand-binding domain may be chosen to recognize a ligand that acts as a cell surface marker on target cells, particularly “off target” cells.

The present invention particularly aims to avoid the “off target” events, wherein engineered immune cells target not only pathological cells, in particular cancerous cells, in particularly due to lack of specificity of the antigen (the latter being present on the pathological (e.g., cancerous) cells but can also be present on healthy, non-pathological cells).

By “cancerous cells”, it is meant cells differing from normal, healthy cells in many ways that allow them to grow faster and longer than healthy cells and become invasive. Cancer cells are less specialized than normal cells and continue to divide without stopping.

Therefore, the extracellular ligand-binding domain of the cell death inducing CAR within the scope of the invention may be chosen in such a way that the cell death inducing CAR recognizes off-target cells (healthy cells), while an extracellular ligand-binding domain of an activating receptor, such as an activating chimeric antigen receptor, recognizes on-target cells (i.e. pathological, e.g., cancerous, cells). Thus, when the engineered immune cell encounters a pathological (e.g., cancerous) cell, only the activating receptor is able to bind to it and not the cell death inducing CAR, and consequently the activating receptor can activated the immune cell to kill the pathological, e.g., cancerous, cell. Conversely, when an engineered immune cell encounters a normal, non-pathological cell, the cell death inducing CAR is able to bind to it, which binding will then trigger the cell death of the engineered immune cell. In consequence, the healthy, non-pathological (e.g., cancerous) cell will be preserved.

According to certain embodiments, the extracellular binding domain of the cell death inducing CAR is specific for an off-target antigen. According to particular embodiments, the off-target antigen is not present or present at low level on a pathological (e.g., cancerous) cell.

According to certain embodiments, the extracellular binding domain of the cell death inducing CAR is specific for an off-target antigen. According to particular embodiments, the off-target antigen is not present or present at low level on a pathological (e.g., cancerous cell) targeted by an activating receptor, such as an activating CAR, as detailed herein.

According to certain embodiments, the extracellular binding domain of the cell death inducing CAR is specific for a cell surface antigen N, N being present on a non-pathological (e.g., non-cancerous) or healthy cell, but not present or present at low level on a pathological (e.g., cancerous) cell as determined by FACS or western blot analysis or by any appropriate technique allowing proteins to be quantified.

“Present at low level” on the pathological (e.g., cancerous) cells means that the expression of said off-tissue antigen is undetectable in tumor cells using any known technique of antigen detection (e.g., flow cytometry, immunohistochemistry, western blot) or represents less than 10% expression as compared to expression in a cell or a tissue used as a positive control (e.g. a non-pathological (e.g., cancerous) cell).

According to certain embodiments, the extracellular binding domain of the cell death inducing CAR is specific for a cell surface antigen N, N being expressed on a non-pathological or healthy cell, but not being expressed on a pathological (e.g., cancerous) cell.

According to certain embodiments, the extracellular binding domain of the cell death inducing CAR is specific for a cell surface antigen N, N being expressed on a non-pathological or healthy cell, but not being expressed on a pathological (e.g., cancerous) cell targeted by an activating receptor, such as an activating CAR, as detailed herein.

The below table provides examples of combinations of antigens recognized by activating CARs and cell death inducing CARs of the invention, respectively.

| Antigen | Antigen |
|---|---|
| specificity of | specificity of |
| activating | cell death |
| receptor | inducing CAR |
| CD38 | CD56 antigen: expression on the surface of neurons, |
| | glia, skeletal muscle and natural killer cells |
| | CD205 antigen: expression on cortical thymic epithelial |
| | cells and by dendritic cell (DC) subsets |
| | CD83 antigen: expression on activated lymphocytes, |
| | Langerhans cells and interdigitating reticulum cells |
| | CD206 antigen: expression on the surface of macrophages |
| | and dendritic cells, on the surface of skin cells such as |
| | human dermal fibroblasts and keratinocytes |
| | CD200 antigen: expression on cells originating from |
| | the hematopoietic cells, activated T cells, endothelial |
| | neuronal cells and cells of the reproductive organs |
| | (ovaries and placental trophoblasts) |
| | CD36 antigen: expression in adipocytes endothelial |
| | cells and monocytes |
| | RARRES1 antigen: expression of this gene upregulated |
| | by tazarotene as well as by retinoic acid receptors |
| CS1 | Troponin C antigen: expression in heart |
| | Beta-1 integrin antigen: expression in endothelial cells |
| | and fibroblasts (at protein level). Expression in intestine, |
| | colon, testis, ovary, thymus, spleen and prostate |
| | CCKBR antigen: expression in stomach, pancreas, |
| | brain and gallbladder |
| | GALR1 antigen: expression in adrenal gland |
| | CUBN antigen: expression in kidney and small intestine |
| CD123 | CD4 antigen: expression in appendix, bone marrow, |
| | lymph node, tonsil and spleen |
| | CD20 antigen: expression mainly in spleen appendix and |
| | lymph node |
| | CD22 antigen: expression in particular in appendix, |
| | lymph node, tonsil and spleen |
| | CD25 antigen: expression mainly in bladder and lymph |
| | node |
| | MUC1 antigen: expression in kidney |
| ROR1 | Troponin C antigen: expression in heart |
| | Beta-1 integrin antigen: expression in endothelial cells and |
| | fibroblasts (at protein level). Expression in intestine, colon, |
| | testis, ovary, thymus, spleen and prostate |
| | CCKBR antigen: expression in stomach, pancreas, brain and |
| | gallbladder |
| | GALR1 antigen: expression in adrenal gland |
| | MUC1 antigen: expression in kidney |
| CD33 | Antigens specifically expressed in dendritic cells and/or |
| FLT3 | haematopoetic stem cells such as ITGAX, CD1E, CD34, |
| | CD1C, CD123, CD141 |
| | Antigens specifically expressed in haematopoetic stem cells |
| | such as CD34 or specifically expressed in Brain cerebellum |
| | such as ZP2, GABRA6, CRTAM, GRM4, MDGA1 |
| MSLN | Antigens specifically expressed in lung such as SFTPC, |
| | ROS1, SLC6A4, AGTR2 |
| MUC16 | Antigens specifically expressed in salivary gland such |
| MUC17 | as LRRC26, HTR3A, TMEM211, MRGPRX3 |
| | Antigens specifically expressed in colon & small intestine |
| | such as MEP1B, TMIGD1, CEACAM20, ALPI |

According to certain embodiments, the extracellular ligand-binding domain of the cell death inducing CAR is specific for a target antigen (e.g., cell surface antigen) selected from the group consisting of: CD123; CD19; CD22; CD30; CD70; CD171; CS-1 (also referred to as CD2 subset 1, CRACC, SLAMF7, CD319, and 19A24); DLL3; TSPAN10; PRAME; C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-I IRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1 (MUC1); Mucin 16 (MUC16); Mucin 17 (MUC17); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAFX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(I-4)bDGlcp(I-I)Cer); transglutaminase 5 (TGS5); high molecular weight-melanoma-associated antigen (HMWMAA); o-acetyl-GD2 (OAcGD2); Folate receptor beta; tumor endothelial marker 1 (TEM1/CD248); tumor endothelial marker 7-related (TEM7R); claudin 6 (CLDN6); claudin 18 (CLDN18), including splice variant 2 (claudin18.2); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Lymphocyte antigen 6 complex locus protein G6d (LY6G6D); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70 (HSP70); heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), CD56, CD205, CD83, CD206, CD200, CD36, RARRES1, Troponin C, Beta-1 integrin, CCKBR, GALR1, CD4, CD20, CD22, CD25, CD34, MUC1.

According to certain embodiments, the extracellular ligand-binding domain of the cell death inducing CAR is specific for an antigen selected from the group consisting of CD56, CD205, CD83, CD206, CD200, CD36, RARRES1, Troponin C, Beta-1 integrin, CCKBR, GALR1, CD4, CD20, CD22, CD25, CD34, MUC1 and EGFRVIII.

According to certain embodiments, the extracellular ligand-binding domain of the cell death inducing CAR is specific for an antigen selected from the group consisting of CD56, CD205, CD83, CD206, CD200, CD36, RARRES1, Troponin C, Beta-1 integrin, CCKBR, GALR1, CD4, CD20, CD22, CD25 and MUC1.

According to certain embodiments, the extracellular ligand-binding domain of the cell death inducing CAR is specific for an antigen selected from the group consisting of CD19, CD3 and CD20, and the extracellular ligand-binding domain of the activating CAR is specific for CD22.

According to particular embodiments, the extracellular ligand-binding domain of the cell death inducing CAR is specific for CD56. Such extracellular ligand-binding domain may be a scFV derived from a monoclonal CD56 antibody, such as a scFV derived from a human or humanized monoclonal CD56 antibody.

According to particular embodiments, the extracellular ligand-binding domain of the cell death inducing CAR is specific for CD205. Such extracellular ligand-binding domain may be a scFV derived from a monoclonal CD205 antibody, such as a scFV derived from a human or humanized monoclonal CD205 antibody.

According to particular embodiments, the extracellular ligand-binding domain of the cell death inducing CAR is specific for CD83. Such extracellular ligand-binding domain may be a scFV derived from a monoclonal CD83 antibody, such as a scFV derived from a human or humanized monoclonal CD83 antibody.

According to particular embodiments, the extracellular ligand-binding domain of the cell death inducing CAR is specific for CD206. Such extracellular ligand-binding domain may be a scFV derived from a monoclonal CD206 antibody, such as a scFV derived from a human or humanized monoclonal CD206 antibody.

According to particular embodiments, the extracellular ligand-binding domain of the cell death inducing CAR is specific for CD200. Such extracellular ligand-binding domain may be a scFV derived from a monoclonal CD200 antibody, such as a scFV derived from a human or humanized monoclonal CD200 antibody.

According to particular embodiments, the extracellular ligand-binding domain of the cell death inducing CAR is specific for CD36. Such extracellular ligand-binding domain may be a scFV derived from a monoclonal CD36 antibody, such as a scFV derived from a human or humanized monoclonal CD36 antibody.

According to particular embodiments, the extracellular ligand-binding domain of the cell death inducing CAR is specific for RARRES1. Such extracellular ligand-binding domain may be a scFV derived from a monoclonal RARRES1 antibody, a scFV derived from a human or humanized monoclonal RARRES1 antibody.

According to particular embodiments, the extracellular ligand-binding domain of the cell death inducing CAR is specific for is specific for Troponin C. Such extracellular ligand-binding domain may be a scFV derived from a monoclonal Troponin C antibody, such as a scFV derived from a human or humanized monoclonal Troponin C antibody.

According to particular embodiments, the extracellular ligand-binding domain of the cell death inducing CAR is specific for Beta-1 integrin. Such extracellular ligand-binding domain may be a scFV derived from a monoclonal Beta-1 integrin antibody, such as a scFV derived from a human or humanized monoclonal Beta-1 integrin antibody.

According to particular embodiments, the extracellular ligand-binding domain of the cell death inducing CAR is specific for CCKBR. Such extracellular ligand-binding domain may be a scFV derived from a monoclonal CCKBR antibody, such as a scFV derived from a human or humanized monoclonal CCKBR antibody.

According to particular embodiments, the extracellular ligand-binding domain of the cell death inducing CAR is specific for GALR1. Such extracellular ligand-binding domain may be a scFV derived from a monoclonal GALR1 antibody, such as a scFV derived from a human or humanized monoclonal GALR1 antibody.

According to particular embodiments, the extracellular ligand-binding domain of the cell death inducing CAR is specific for CD4. Such extracellular ligand-binding domain may be a scFV derived from a monoclonal CD4 antibody, such as a scFV derived from a human or humanized monoclonal CD4 antibody.

According to particular embodiments, the extracellular ligand-binding domain of the cell death inducing CAR is specific for CD20. Such extracellular ligand-binding domain may be a scFV derived from a monoclonal CD20 antibody, such as a scFV derived from a human or humanized monoclonal CD20 antibody.

According to certain embodiments, the extracellular ligand-binding domain of the cell death inducing CAR is not specific for CD20.

According to particular embodiments, the extracellular ligand-binding domain of the cell death inducing CAR is specific CD22. Such extracellular ligand-binding domain may be a scFV derived from a monoclonal CD22 antibody, such as a scFV derived from a human or humanized monoclonal CD22 antibody.

According to certain embodiments, the extracellular ligand-binding domain of the cell death inducing CAR is not specific for CD22.

According to particular embodiments, the extracellular ligand-binding domain of the cell death inducing CAR is specific for CD25. Such extracellular ligand-binding domain may be a scFV derived from a monoclonal CD25 antibody, such as a scFV derived from a human or humanized monoclonal CD25 antibody.

According to particular embodiments, the extracellular ligand-binding domain of the cell death inducing CAR is specific for MUC1. Such extracellular ligand-binding domain may be a scFV derived from a MUC1 monoclonal antibody, such as a scFV derived from a human or humanized monoclonal MUC1 antibody.

According to certain embodiments, the extracellular ligand-binding domain of the cell death inducing CAR is not specific for MUC1.

According to particular embodiments, the extracellular ligand-binding domain of the cell death inducing CAR is specific for CD34. Such extracellular ligand-binding domain may be a scFV derived from a CD34 monoclonal antibody, such as a scFV derived from a human or humanized monoclonal CD34 antibody.

According to particular embodiments, the extracellular ligand-binding domain of the cell death inducing CAR is specific for EGFRVIII. Such extracellular ligand-binding domain may be a scFV derived from a EGFRVIII monoclonal antibody, such as a scFV derived from a human or humanized monoclonal EGFRVIII antibody.

The extracellular ligand-binding domain may comprise an antigen binding fragment derived from an antibody, such as human or humanized antibody, against an antigen of the target. Thus, according to certain embodiments, the extracellular ligand-binding domain is an extracellular antigen binding domain. According to particular embodiments, the extracellular antigen binding domain comprises an antibody or antigen binding fragment thereof. The antigen binding fragment may be, for example, a scFv or a Fab.

According to particular embodiments, the extracellular antigen-binding domain is a scFv, preferably one derived from a monoclonal antibody against an antigen of a target. The monoclonal antibody may be human in origin or may have been humanized. More specifically, the extracellular antigen-binding domain may comprise a single chain antibody fragment (scFv) comprising the light (VL) and the heavy (VH) variable fragment of a target antigen specific monoclonal antibody, optionally joined by a peptide linker composed of, e.g., 5 to 25 amino acids. The linker sequence may comprise any naturally occurring amino acid. The linker sequence may comprise amino acids glycine and serine. The linker sequence may comprise sets of glycine and serine repeats such as (Gly4Ser)n, where n is a positive integer equal to or greater than 1 (such as a GGGGSGGGGSGGGGS-linker as shown in SEQ ID NO: 37).

According to particular embodiments, the extracellular antigen binding domain is a Fab, preferably one derived from a monoclonal antibody against an antigen of a target. The monoclonal antibody may be human in origin or may have been humanized.

It is also contemplated by the present invention that the extracellular ligand-binding domain of the cell death inducing CAR is specific for a ligand which does not act as a cell surface marker on target cells. Instead, the ligand could be a small molecule, i.e. an organic compound having a low molecular weight (<2000 daltons). Binding of the extracellular ligand-binding domain to said small molecule will then induce cell death of the engineered immune cell endowed with the cell death inducing CAR.

Therefore, according to certain embodiments, the extracellular ligand-binding domain of the cell death inducing CAR is specific for a small molecule.

Generally, binding of the extracellular ligand-binding domain of the cell death inducing CAR to its target ligand will trigger cell death of the cell expressing said CAR on its surface.

According to certain embodiments, the cell death-inducing CAR induces cell death upon binding of said cell death domain-inducing CAR to its target antigen, only.

According to certain embodiments, the cell death-inducing CAR induces cell death upon binding of said cell death domain-inducing CAR to one of the targets it binds to.

According to certain embodiments, the present invention relates to cell death inducing chimeric antigen receptors, wherein the ectodomain further comprises at least one specific epitope such as a monoclonal antibody (mAb)-specific epitope. Such mAb-specific epitope allows both sorting and/or depletion of immune cells endowed with such cell death inducing CAR(s).

According to certain embodiments, two mAb-specific epitopes are inserted.

The epitope(s) may be inserted anywhere in the ectodomain, either in the N terminal part, e.g., between the VH and VL chains of the scFvs, or between the hinge and extracellular ligand binding domain. Preferably, when more than one mAb-specific epitope are used, they are not in tandem (side by side).

According to certain embodiments, the epitope is a mimotope. As a macromolecule, often a peptide, which mimics the structure of an epitope, the mimotope has the advantage to be smaller than a conventional epitope, and therefore may be beneficial for a non-conformational sequence and easier to reproduce in a long polypeptide such a CAR. Mimotopes are known for several pharmaceutically-approved mAb such as two 10 amino acid peptides for cetuximab (Riemer et al., 2005), or a 24 aa for palivizumab (Arbiza et al, 1992). As these mimotopes can be identified by phage display, it is possible to try several of them in order to obtain a sequence which does not perturb the scFv for the same mAb. Furthermore, their use can enhance a complement-dependent cytotoxicity (CDC).

Non-limiting of mimotopes that may be employed include mimotopes of CD20 (e.g., SEQ ID NO: 38), mimotopes corresponding to the use of cetuximab (e.g., SEQ ID NO: 39; SEQ ID NO: 40; SEQ ID NO: 41; SEQ ID NO: 42); mimotopes corresponding to the use of palivizumab (e.g., of SEQ ID NO: 43) and mimotopes corresponding to the use of nivolumab (e.g., SEQ ID NO: 44; SEQ ID NO: 45). Further non-limited examples of suitable mimotopes that may be employed are described in, e.g., WO2016/120216. Preferred constructions comprise 1, 2 or 3 consecutive or distant CD20 mimotopes and an additional CD34 epitope, while more preferred constructions comprises from N-terminus to C-terminus: a CD20 mimotope, a VH, a spacer, a VL, and a hinge comprising a CD20 mimotope, a CD34 epitope and another CD20 mimotope.

According to certain embodiments, the ectodomain of the cell death inducing CAR comprises at least one monoclonal antibody (mAb)-specific epitope (such as a mimotope) which is recognized by an monoclonal antibody selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

According to certain embodiments, the epitope is recognized by rituximab. According to certain embodiments, the epitope is recognized by QBEND-10.

According to certain embodiments, the binding of the mAB to the mAB-specific epitope induces cell death. In certain embodiment cell death occurs upon binding of CD34 epitope and/or CD20 mimotope to its specific ligand in the CAR.

A cell death inducing chimeric antigen receptor according to the present invention may be a single chain CAR. A single chain CAR is a chimeric antigen receptor wherein all domains of which said CAR is composed are located on one polypeptide chain.

Alternatively, a cell death inducing chimeric antigen receptor according to the present invention may be a multi-chain CAR. According to this architecture, at least on ectodomain and the at least one endodomain are born on different polypeptide chains. The different polypeptide chains are anchored into the membrane in a close proximity allowing interactions with each other. The multi-subunit architecture also offers more flexibility and possibilities of designing cell death inducing CARs. For instance, it is possible to include several extracellular ligand-binding domains having different specificity to obtain a multi-specific cell death inducing CAR architecture. This type of architecture has been recently described by the applicant in PCT/US2013/058005.

Accordingly, a multi-chain cell death inducing CAR according to the invention may be one which comprises:
A) a first polypeptide chain comprising
   a) at least one ectodomain which comprises an extracellular ligand-binding domain and a hinge; and
   aa) at least one transmembrane domain; and
B) a second polypeptide chain comprising
   b) at least one endodomain comprising a death domain; and
   bb) at least one transmembrane domain.

The assembly of the different chains as part of a single multi-chain cell death inducing CAR is made possible, for instance, by using the different alpha, beta and gamma chains of the high affinity receptor for IgE (FcεRI).

Thus, according to certain embodiments, the first polypeptide chain (A) comprising the ectodomain comprises the transmembrane domain from the alpha chain of high-affinity IgE receptor (FcεRI), whereas the second polypeptide chain (B) comprising the endodomain which comprises the death domain comprises the transmembrane domain from the gamma or beta chain of FcεRI, such as the transmembrane domain from the gamma chain of FcεRI.

### Activating Receptor/Activating CAR

As mentioned above, the present invention relates to “logical NOT” gates that involve, beside the above described cell death inducing CAR, at least one activating receptor, such as an activating chimeric antigen receptor. The activating receptor enables an engineered immune cell to trigger the destruction of pathologic targeted cells, such as pathological (e.g., cancerous) cells.

According to certain embodiments, the activating receptor is a recombinant T cell receptor.

According to certain embodiments, the activating receptor is an activating chimeric antigen receptor (A-CAR), which may generally be characterized to comprise:
a) at least one ectodomain which comprises an extracellular ligand-binding domain and optionally a hinge;
b) at least one transmembrane domain; and
c) at least one endodomain which comprises a signal transducing domain and optionally a co-stimulatory domain.

The “extracellular ligand-binding domain” comprised by the ectodomain of the activating receptor, such as an activating chimeric antigen receptor, may be any oligo- or polypeptide that is capable of binding a ligand, more specifically an antigen. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells. In particular, the extracellular ligand-binding domain can comprise an antigen binding fragment derived from an antibody, such as a human or humanized antibody, against an antigen of the target.

Thus, according to certain embodiments, the extracellular ligand-binding domain comprises an antibody or antigen binding fragment thereof. The antigen binding fragment may be, for example, a scFv or a Fab.

According to particular embodiments, the extracellular-ligand binding domain is a scFv, preferably one derived from a monoclonal antibody against an antigen of a target. The monoclonal antibody may be human in origin or may have been humanized. More specifically, the extracellular ligand-binding domain may comprise a single chain antibody fragment (scFv) comprising the light (VL) and the heavy (VH) variable fragment of a target antigen specific monoclonal antibody, optionally joined by a peptide linker composed of, e.g., 5 to 25 amino acids. The linker sequence may comprise any naturally occurring amino acid. The linker sequence may comprise amino acids glycine and serine. The linker sequence may comprise sets of glycine and serine repeats such as (Gly4Ser)n, where n is a positive integer equal to or greater than 1 (such as a GGGGSGGGGSGGGGS-linker as shown in SEQ ID NO: 37).

According to other particular embodiments, the extracellular antigen binding domain is a Fab, preferably one derived from a monoclonal antibody against an antigen of a target. The monoclonal antibody may be human in origin or may have been humanized.

As non-limiting examples, the antigen of the target can be any cluster of differentiation molecules (e.g. CD16, CD64, CD78, CD96, CD56, CD116, CD117, CD71, CD45, CD71, CD123 and CD138), Human C-type lectin-like molecule-1 (CLL1), a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, 13-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), or a virus-specific surface antigen such as an HIV-specific antigen (such as HIV gp120); an HBV-specific antigen, an EBV-specific antigen, a CMV-specific antigen, a HPV-specific antigen, a Lasse Virus-specific antigen, an Influenza Virus-specific antigen, a fungi-specific antigen or a bacterium-specific antigen as well as any derivate or variant of these surface markers. Antigens are not necessarily surface marker antigens but can be also endogenous small antigens presented by HLA class I at the surface of the cells.

According to certain embodiments, the extracellular ligand-binding domain of the activating receptor, such as activating CAR, is specific for a cell surface antigen P, P being expressed or over-expressed on a targeted pathological (e.g., cancerous) cell.

According to certain embodiments, the extracellular ligand-binding domain of the activating receptor, such as activating CAR, is specific for a cell surface antigen P, wherein P is different to a cell surface antigen N for which the extracellular ligand-binding domain of the cell death inducing chimeric antigen is specific.

According to certain embodiments, the extracellular ligand-binding domain of the activating receptor, such as activating CAR, is specific for a cell surface antigen P, wherein P is a cell surface antigen not recognized by the extracellular ligand-binding domain of the cell death inducing chimeric receptor.

According to certain embodiment, the extracellular ligand-binding domain of the activating receptor and the extracellular ligand-binding domain of the cell death inducing chimeric receptor are specific for the same target antigen (e.g., cell surface antigen).

According to certain embodiment, the extracellular ligand-binding domain of the activating receptor and the extracellular ligand-binding domain of the cell death inducing chimeric receptor are specific for different target antigens (e.g., cell surface antigens).

According to certain embodiments, the extracellular-ligand binding domain is specific for a target antigen (e.g., cell surface antigen) selected from the group consisting of: CD123; CD19; CD22; CD30; CD70; CD171; CS-1 (also referred to as CD2 subset 1, CRACC, SLAMF7, CD319, and 19A24); DLL3; TSPAN10; PRAME; C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-I IRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1 (MUC1); Mucin 16 (MUC16); Mucin 17 (MUC17); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAFX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(I-4)bDGlcp(I-I)Cer); transglutaminase 5 (TGS5); high molecular weight-melanoma-associated antigen (HMWMAA); o-acetyl-GD2 (OAcGD2); Folate receptor beta; tumor endothelial marker 1 (TEM1/CD248); tumor endothelial marker 7-related (TEM7R); claudin 6 (CLDN6); claudin 18 (CLDN18), including splice variant 2 (claudin18.2); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Lymphocyte antigen 6 complex locus protein G6d (LY6G6D); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-Ia); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70 (HSP70); heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

According to certain embodiments, the extracellular-ligand binding domain is specific for a target antigen selected from the group consisting of: CD123, ROR1, BCMA, PSMA, CD33, CD38, CD22, CS1, CLL-1, HSP70, EGFRVIII, FLT3, WT1, CD30, CD70, MUC1, MUC16, MUC17, PRAME, TSPAN10, Claudin18.2, DLL3, LY6G6D and o-acetyl-GD2 (OAcGD2).

According to certain embodiments, the extracellular-ligand binding domain is specific for a target antigen selected from the group consisting of: CD123, CD38, CD22, CS1, CLL-1, HSP70, CD30, MUC1 and o-acetyl-GD2 (OAcGD2).

According to certain embodiments, the extracellular ligand-binding domain is specific for CD123. Such extracellular ligand-binding domain may be a scFV derived from a monoclonal CD123 antibody, such as a scFV derived from a human or humanized monoclonal CD123 antibody.

According to other certain embodiments, the extracellular ligand-binding domain is specific for ROR1. Such extracellular ligand-binding domain may be a scFV derived from a monoclonal ROR1 antibody, such as a scFV derived from a human or humanized monoclonal ROR1 antibody.

According to other certain embodiments, the extracellular ligand-binding domain is specific for BCMA. Such extracellular ligand-binding domain may be a scFV derived from a monoclonal BCMA antibody, such as a scFV derived from a human or humanized monoclonal BCMA antibody.

According to other certain embodiments, the extracellular ligand-binding domain is specific for PSMA. Such extracellular ligand-binding domain may be a scFV derived from a monoclonal PSMA antibody, such as a scFV derived from a human or humanized monoclonal PSMA antibody.

According to other certain embodiments, the extracellular ligand-binding domain is specific for CD33. Such extracellular ligand-binding domain may be a scFV derived from a monoclonal CD33 antibody, such as a scFV derived from a human or humanized monoclonal CD33 antibody.

According to other certain embodiments, the extracellular ligand-binding domain is specific for CD38. Such extracellular ligand-binding domain may be a scFV derived from a monoclonal CD38 antibody, such as a scFV derived from a human or a humanized monoclonal CD38 antibody.

According to other certain embodiments, the extracellular ligand-binding domain is specific for CS1. Such extracellular ligand-binding domain may be a scFV derived from a monoclonal CS1 antibody, such as a scFV derived from a human or humanized monoclonal CS1 antibody.

According to other certain embodiments, the extracellular ligand-binding domain is specific for CLL-1. Such extracellular ligand-binding domain may be a scFV derived from a monoclonal CLL-1 antibody, such as a scFV derived from a human or humanized monoclonal CLL-1 antibody.

According to other certain embodiments, the extracellular ligand-binding domain is specific for HSP70. Such extracellular ligand-binding domain may be a scFV derived from a monoclonal HSP70 antibody, such as a scFV derived from a human or humanized monoclonal HSP70 antibody.

According to certain embodiments, the extracellular ligand-binding domain is specific for EGFRVIII. Such extracellular ligand-binding domain may be a scFV derived from a monoclonal EGFRVIII antibody, such as a scFV derived from a human or humanized monoclonal EGFRVIII antibody.

According to other certain embodiments, the extracellular ligand-binding domain is specific for FLT3. Such extracellular ligand-binding domain may be a scFV derived from a monoclonal FLT3 antibody, such as a scFV derived from a human or humanized monoclonal FLT3 antibody.

According to certain embodiments, the extracellular ligand-binding domain is specific for WT1. Such extracellular ligand-binding domain may be a scFV derived from a monoclonal WT1 antibody, such as a scFV derived from a human or humanized monoclonal WT1 antibody.

According to certain embodiments, the extracellular ligand-binding domain is specific for CD30. Such extracellular ligand-binding domain may be a scFV derived from a monoclonal CD30 antibody, such as a scFV derived from a human or humanized monoclonal CD30 antibody.

According to certain embodiments, the extracellular ligand-binding domain is specific for CD70. Such extracellular ligand-binding domain may be a scFV derived from a monoclonal CD70 antibody, such as a scFV derived from a human or humanized monoclonal CD70 antibody.

According to certain embodiments, the extracellular ligand-binding domain is specific for MUC16. Such extracellular ligand-binding domain may be a scFV derived from a monoclonal MUC16 antibody, such as a scFV derived from a human or humanized monoclonal MUC16 antibody.

According to certain embodiments, the extracellular ligand-binding domain is specific for MUC17. Such extracellular ligand-binding domain may be a scFV derived from a monoclonal MUC17 antibody, such as a scFV derived from a human or humanized monoclonal MUC17 antibody.

According to certain embodiments, the extracellular ligand-binding domain is specific for PRAME. Such extracellular ligand-binding domain may be a scFV derived from a monoclonal PRAME antibody, such as a scFV derived from a human or humanized monoclonal PRAME antibody.

According to certain embodiments, the extracellular ligand-binding domain is specific for TSPAN10. Such extracellular ligand-binding domain may be a scFV derived from a monoclonal TSPAN10 antibody, such as a scFV derived from a human or humanized monoclonal TSPAN10 antibody.

According to certain embodiments, the extracellular ligand-binding domain is specific for Claudin18.2. Such extracellular ligand-binding domain may be a scFV derived from a monoclonal Claudin18.2 antibody, such as a scFV derived from a human or humanized monoclonal Claudin18.2 antibody.

According to certain embodiments, the extracellular ligand-binding domain is specific for DLL3. Such extracellular ligand-binding domain may be a scFV derived from a monoclonal DLL3 antibody, such as a scFV derived from a human or humanized monoclonal DLL3 antibody.

According to certain embodiments, the extracellular ligand-binding domain is specific for LY6G6D. Such extracellular ligand-binding domain may be a scFV derived from a monoclonal LY6G6D antibody, such as a scFV derived from a human or humanized monoclonal LY6G6D antibody.

According to certain embodiments, the extracellular ligand-binding domain is specific for GD2. Such extracellular ligand-binding domain may be a scFV derived from a monoclonal GD2 antibody, such as a scFV derived from a human or humanized monoclonal GD2 antibody.

According to certain embodiments, the extracellular ligand-binding domain is specific for o-acetyl-GD2 (OAcGD2). Such extracellular ligand-binding domain may be a scFV derived from a monoclonal OAcGD2 antibody, such as a scFV derived from a human or humanized monoclonal OAcGD2 antibody.

An activating chimeric antigen receptor according to the present invention may comprise two extracellular ligand-binding domains.

An activating chimeric antigen receptor according to the present invention may further comprise a hinge within the at least one ectodomain. The hinge is suitably located between the extracellular ligand-binding domain and the transmembrane domain.

As mentioned above, the term “hinge” or “hinge region” used herein generally means any oligo- or polypeptide that functions to link the transmembrane domain to the switch domain. In particular, a hinge is used to provide more flexibility and accessibility for the extracellular ligand-binding domain. A hinge region may comprise up to 300 amino acids, preferably 10 to 100 amino acids and most preferably 25 to 50 amino acids. A hinge may be derived from all or part of naturally occurring molecules, such as from all or part of the extracellular region of CD8, CD4 or CD28, or from all or part of an antibody constant region. Alternatively the hinge may be a synthetic sequence that corresponds to a naturally occurring hinge sequence, or may be an entirely synthetic hinge sequence. Non-limiting examples of hinges which may be used in accordance to the invention include a part of human CD8 alpha chain, FcγRIIIα receptor or IgG1.

According to certain embodiments, the hinge is selected from the group consisting of CD8a hinge, IgG1 hinge and FcγRIIIα hinge. According to particular embodiments, the hinge is selected from the group consisting of IgG1 hinge and FcγRIIIα hinge. According to more particular embodiments, the hinge is a IgG1 hinge.

According to certain embodiments, the hinge is a CD8a hinge. According to particular embodiments, the hinge comprises, or consists of, an amino acid sequence having at least 80%, such as at least 85%, sequence identity with SEQ ID NO: 22. According to particular embodiments, the hinge comprises, or consists of, an amino acid sequence having at least 90%, such as at least 95%, sequence identity with SEQ ID NO: 22.

According to certain embodiments, the hinge is a IgG1 hinge. According to particular embodiments, the hinge comprises, or consists of, an amino acid sequence having at least 80%, such as at least 85%, sequence identity with SEQ ID NO: 23. According to particular embodiments, the hinge comprises, or consists of, an amino acid sequence having at least 90%, such as at least 95%, sequence identity with SEQ ID NO: 23.

According to certain embodiments, the hinge is a FcγRIIIα hinge. According to particular embodiments, the hinge comprises, or consists of, an amino acid sequence having at least 80%, such as at least 85%, sequence identity with SEQ ID NO: 24. According to particular embodiments, the hinge comprises, or consists of, an amino acid sequence having at least 90%, such as at least 95%, sequence identity with SEQ ID NO: 24.

Preferred activating CARs of the invention comprise an extracellular ligand-binding domain specific for any one of a target antigen described above in combination, e.g. fused with, a IgG1 hinge.

An activating chimeric antigen receptor according to the invention comprises at least one transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The at least one transmembrane domain can be derived either from a natural or from a synthetic source. The at least one transmembrane domain can be derived from any membrane-bound or transmembrane protein. As non-limiting examples, the at least one transmembrane domain can be a subunit of the T cell receptor such as α, β, γ or δ, polypeptide constituting CD3 complex, IL2 receptor p55 (a chain), p75 (β chain) or γ chain, a subunit chain of Fc receptors, in particular Fcγ receptor III or CD proteins. Alternatively, the at least one transmembrane domain can be synthetic and can comprise predominantly hydrophobic residues such as leucine and valine.

According to certain embodiments, the transmembrane domain is selected from the group consisting of the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI Ia, CD18), ICOS (CD278), 4-1BB (CD137), GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, LFA-1, ITGAM, CDI Ib, ITGAX, CDI Ic, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D or NKG2C.

According to certain embodiments, the at least one transmembrane domain is selected from the group consisting of CD8 alpha transmembrane domain, 4-1BB transmembrane domain, DAP10 transmembrane domain and CD28 transmembrane domain.

According to certain embodiments, the at least one transmembrane domain is a CD8 alpha transmembrane domain. According to particular embodiments, the at least one transmembrane domain comprises, or consists of, an amino acid sequence having at least 80%, such as at least 85%, sequence identity with SEQ ID NO: 30. According to particular embodiments, the hinge comprises, or consists of, an amino acid sequence having at least 90%, such as at least 95%, sequence identity with SEQ ID NO: 30.

According to certain embodiments, the at least one transmembrane domain is a 4-1BB transmembrane domain. According to particular embodiments, the at least one transmembrane domain comprises, or consists of, an amino acid sequence having at least 80%, such as at least 85%, sequence identity with SEQ ID NO: 31. According to particular embodiments, the hinge comprises, or consists of, an amino acid sequence having at least 90%, such as at least 95%, sequence identity with SEQ ID NO: 31.

According to certain embodiments, the at least one transmembrane domain is a DAP10 transmembrane domain. According to particular embodiments, the at least one transmembrane domain comprises, or consists of, an amino acid sequence having at least 80%, such as at least 85%, sequence identity with SEQ ID NO: 32. According to particular embodiments, the hinge comprises, or consists of, an amino acid sequence having at least 90%, such as at least 95%, sequence identity with SEQ ID NO: 32.

According to certain embodiments, the at least one transmembrane domain is a CD28 transmembrane domain. According to particular embodiments, the at least one transmembrane domain comprises, or consists of, an amino acid sequence having at least 80%, such as at least 85%, sequence identity with SEQ ID NO: 33. According to particular embodiments, the hinge comprises, or consists of, an amino acid sequence having at least 90%, such as at least 95%, sequence identity with SEQ ID NO: 33.

According to certain embodiments, the at least one transmembrane domain is selected from the group consisting of the transmembrane domains of the FcεRI α, β and γ chains. According to certain embodiments, the at least one transmembrane domain is selected from the group consisting of the transmembrane domains of the human FcεRI α, β and γ chains. According to particular embodiments, the at least one transmembrane domain comprises, or consists of, an amino acid sequence having at least 80%, such as at least 85%, sequence identity with SEQ ID NO: 34. According to particular embodiments, the hinge comprises, or consists of, an amino acid sequence having at least 90%, such as at least 95%, sequence identity with SEQ ID NO: 34. According to particular embodiments, the at least one transmembrane domain comprises, or consists of, an amino acid sequence having at least 80%, such as at least 85%, sequence identity with SEQ ID NO: 35. According to particular embodiments, the hinge comprises, or consists of, an amino acid sequence having at least 90%, such as at least 95%, sequence identity with SEQ ID NO: 35. According to particular embodiments, the at least one transmembrane domain comprises, or consists of, an amino acid sequence having at least 80%, such as at least 85%, sequence identity with SEQ ID NO: 36. According to particular embodiments, the hinge comprises, or consists of, an amino acid sequence having at least 90%, such as at least 95%, sequence identity with SEQ ID NO: 36.

In case that the chimeric antigen receptor is a multi-chain CAR it is comprises of, for example, at least two different polypeptide chains, each of which contains at least one transmembrane domain, the transmembrane domains may, for example, be selected from the transmembrane domains of the FcεRI α, β and γ chains, fragments or variants thereof.

An activating chimeric antigen receptor according to the invention comprises at least one endodomain comprising a signal transducing domain and optionally a co-stimulatory domain

The signal transducing domain or intracellular signaling domain of the activating CAR is responsible for intracellular signaling following the binding of the extracellular ligand-binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines.

In the present application, the term “signal transducing domain” refers to the portion of a protein which transduces the effector signal function signal and directs the cell to perform a specialized function.

Preferred examples of signal transducing domain for use in single or multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM which can be employed in accordance with the invention can include those derived from TCRzeta, FcRgamma, FcRbeta, FcRepsilon, CD3gamma, CD3delta, CD3epsilon, CD3 zeta, CD5, CD22, CD79a, CD79b and CD66d.

According to certain embodiments, the signaling domain comprises the CD3zeta signaling domain, or the intracytoplasmic domain of the FcεRI beta or gamma chains.

According to certain embodiments, the signaling domain comprises a CD3 zeta signaling domain. According to particular embodiments, the signaling domain comprises, or consists of, an amino acid sequence having at least 80%, such as at least 85%, sequence identity with SEQ ID NO: 46. According to particular embodiments, the signaling domain comprises, or consists of, an amino acid sequence having at least 90%, such as at least 95%, sequence identity with SEQ ID NO: 46.

According to certain embodiments, the signaling domain comprises the intracytoplasmic domain of the FcεRI beta chain. According to particular embodiments, the signaling domain comprises, or consists of, an amino acid sequence having at least 80%, such as at least 85%, sequence identity with SEQ ID NO: 47. According to particular embodiments, the signaling domain comprises, or consists of, an amino acid sequence having at least 90%, such as at least 95%, sequence identity with SEQ ID NO: 47.

According to certain embodiments, the signaling domain comprises the intracytoplasmic domain of the FcεRI gamma chain. According to particular embodiments, the signaling domain comprises, or consists of, an amino acid sequence having at least 80%, such as at least 85%, sequence identity with SEQ ID NO: 48. According to particular embodiments, the signaling domain comprises, or consists of, an amino acid sequence having at least 90%, such as at least 95%, sequence identity with SEQ ID NO: 48.

According to certain embodiments, the CAR of the present invention comprises in at least one endodomain a co-stimulatory domain.

The co-stimulatory domain may be any cytoplasmic domain of a costimulatory molecule. A co-stimulatory molecule is a cell surface molecule other than an antigen receptor or their ligands that is required for an efficient immune response.

“Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory signal” as used herein refers to a signal, which in combination with primary signal, such as TCR/CD3 ligation, leads to T cell proliferation and/or upregulation or down regulation of key molecules.

The co-stimulatory domain may, for example, be the cytoplasmic domain from a costimulatory molecule selected from CD27, CD28, 4-1BB, OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, and any combination thereof.

Thus, according to certain embodiments, the co-stimulatory domain is a co-stimulatory domain from 4-1BB. According to particular embodiments, the co-stimulatory domain comprises, or consists of, an amino acid sequence having at least 80%, such as at least 85%, sequence identity with SEQ ID NO: 49. According to particular embodiments, the co-stimulatory domain comprises, or consists of, an amino acid sequence having at least 90%, such as at least 95%, sequence identity with SEQ ID NO: 49.

According to certain embodiments, the present invention provides a CAR comprising at least one endodomain comprising a signal transducing domain, a co-stimulatory domain and at least one death domain. The death domain may be a death domain as described above. Upon binding of the CAR to its target antigen, a signal is transduced to the cell leading to activation, degranulation and ultimately to death of the target pathological cells and of the CAR-expressing immune cell, such as T cell.

According to certain embodiments, the present invention employs an activating CAR, or a cell expressing an A-CAR wherein the ectodomain further comprises at least one specific epitope such as a monoclonal antibody (mAb)-specific epitope. Such specific epitope allows depletion of immune cells endowed with such activating CAR(s).

According to certain embodiments, the present invention employs an activating CAR, wherein the ectodomain further comprises at least one monoclonal antibody (mAb)-specific epitope. Such mAb-specific epitope allows both sorting and/or depletion of immune cells endowed with such activating CAR(s).

According to certain embodiments, two mAb-specific epitopes are inserted.

The epitope(s) may be inserted anywhere in the ectodomain, either in the N terminal part, e.g., between the VH and VL chains of the scFvs, or between the hinge and extracellular ligand binding domain. Preferably, when more than one mAb-specific epitope are used, they are not in tandem (side by side).

According to certain embodiments, the epitope is a mimotope. Non-limiting of mimotopes that may be employed include mimotopes of CD20 (e.g., SEQ ID NO: 38), mimotopes corresponding to the use of cetuximab (e.g., SEQ ID NO: 39; SEQ ID NO: 40; SEQ ID NO: 41; SEQ ID NO: 42); mimotopes corresponding to the use of palivizumab (e.g., of SEQ ID NO: 43) and mimotopes corresponding to the use of nivolumab (e.g., SEQ ID NO: 44; SEQ ID NO: 45). Further non-limited examples of suitable mimotopes that may be employed are described in, e.g., WO2016/120216. Preferred constructions comprise 1, 2 or 3 consecutive or distant CD20 mimotopes and an additional CD34 epitope, while more preferred constructions comprises from N-terminus to C-terminus: a CD20 mimotope, a VH, a spacer, a VL, and a hinge comprising a CD20 mimotope, a CD34 epitope and another CD20 mimotope.

According to certain embodiments, the ectodomain of the activating CAR comprises at least one monoclonal antibody (mAb)-specific epitope (such as a mimotope) which is recognized by an monoclonal antibody selected from the group consisting of ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

According to certain embodiments, the epitope is recognized by rituximab. According to certain embodiments, the epitope is recognized by QBEND-10.

According to certain embodiments, the binding of the mAb to the mAb-specific epitope induces cell death. In certain embodiment cell death occurs upon binding of CD34 epitope and/or CD20 mimotope to its specific ligand in the CAR.

An activating chimeric antigen receptor according to the present invention may be a single chain CAR.

Alternatively, an activating chimeric antigen receptor according to the present invention may be a multi-chain CAR. A multi-chain activating CAR may be derived from any multi chain receptor with a specific (chimeric) extracellular ligand-binding domain, preferably having the structure VH-spacer-VL as described herein. Thus, according to certain embodiments, a multi-chain activating CAR is obtained by replacing the native extracellular ligand binding domain with a non-native extracellular ligand-binding domain.

Accordingly, a multi-chain activating CAR according to the invention may be one which comprises:
A) a first polypeptide chain comprising
   a) at least one ectodomain which comprises an extracellular ligand-binding domain; and
   aa) at least one transmembrane domain; and
B) a second polypeptide chain comprising
   b) at least one endodomain comprising a signal transducing domain and optionally a co-stimulatory domain; and
   bb) at least one transmembrane domain.

According to certain embodiments, a multi-chain activating CAR of the invention may further comprise:
C) a third polypeptide chain comprising
   c) at least one endodomain comprising a co-stimulatory domain; and
   cc) at least one transmembrane domain.

The assembly of the different chains as part of a single multi-chain activating CAR is made possible, for instance, by using the different alpha, beta and gamma chains of the high affinity receptor for IgE (FcεRI). Such multi-chain CARs can be derived from FcεRI, by replacing the high affinity IgE binding domain of FcεRI alpha chain by an ectodomain as detailed above, whereas the N and/or C-termini tails of FcεRI beta and/or gamma chains are fused to an ectodomain as detailed above comprising a signal transducing domain and co-stimulatory domain, respectively. The extracellular ligand binding domain has the role of redirecting T-cell specificity towards cell targets, while the signal transducing domains activate the immune cell response. The fact that the different polypeptide chains derived from the alpha, beta and gamma polypeptides from FcεRI are transmembrane polypeptides sitting in juxtamembrane position, provides a more flexible architecture to CARs, improving specificity towards the antigen target and reducing background activation of immune cells.

Thus, according to particular embodiments, the first polypeptide chain (A) comprising the ectodomain comprises the transmembrane domain from the alpha chain of high-affinity IgE receptor (FcεRI), whereas the second polypeptide chain (B) comprising the endodomain which comprises the signal transducing domain comprises the transmembrane domain from the gamma or beta chain of FcεRI, such as the transmembrane domain from the gamma chain of FcεRI. If present, the third polypeptide chain (C) comprising the endodomain which comprises the co-stimulatory domain comprises the transmembrane domain from the gamma or beta chain of FcεRI, such as the transmembrane domain from the beta chain of FcεRI.

### Polynucleotides, Vectors

The present invention also relates to polynucleotides and vectors that comprise one or more nucleic acid sequences encoding a cell death inducing chimeric antigen receptor according to the invention. Within the scope are also included polynucleotides and vectors that comprise one or more nucleic acid sequences encoding an activating receptor as detailed herein.

The present invention provides polynucleotides, including DNA and RNA molecules, which comprise one or more nucleic acid sequences encoding a cell death inducing chimeric antigen receptor.

The polynucleotide(s) may be comprised by an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In order to drive expression in the host cell, such as an immune cell, the nucleic acid sequence encoding the cell death inducing CAR is operably linked to a promoter, such as an promoter selected from the group consisting of pUBC, pLCK, pEF1a short, pEF1a long, pGK1, pSFFV, SV40, CAG, pTCF7L1, pTCF7L2, pTCF7, and derivatives of any of the aforesaid, preferably selected from the group consisting of pEF1a short, pGK1, and derivatives of any of the aforesaid.

“Derivative” means in the context of a promotor, a promoter having in its sequence one or more mutations, such as substitutions, additions or deletions of one or more nucleotides, that decrease or increase the expression of a reported gene operably linked to said promoter when compared to the wild type (parent) promoter from which it is derived. A derivative may be a truncated version of the wild type (parent) promoter with one or more, e.g., 1, 2, 5, 10, 100, 200, 300, or 400, nucleotide deletions when compared to the wild type promoter sequence. By way of example, derivatives of pGK1 are the promoters pGK100, pGK200, pGK300 and pGK400.

The promoter may also be any promoter of a gene wherein the cell death inducing CAR encoding nucleic acid sequence in integrated to (endogenous promoter of the KO gene eg promoter of the TCR gene). Thus, according to certain embodiments, the promoter is a promoter of a gene wherein the cell death inducing CAR encoding nucleic acid sequence is integrated to (such as an endogenous promoter of a knock-out gene e.g., the promoter of the TCR gene.

As noted above, because of the apoptotic potential conferred by a wild type death domain, unspecific basal cell death might still be seen even in absence of a target for which the cell death inducing CAR shows specificity. Besides have identified and tested beneficial amino acid substitutions within a number of naturally occurring (wild-type) death domains which attenuate self-association of the receptor and/or binding to a pro-apoptotic or pro-necrotic adaptor protein, the present inventors have found that unspecific basal cell death can also be reduced by controlling the level of expression of the cell death inducing CAR.

Controlled expression can, for example, be achieved by employing a promoter allowing an adequate level of expression.

A promoter is defined as adequate if it permits to obtain a level of expression sufficient to (i) specifically deplete the cell death inducing CAR immune cells population using target cells presenting the cell death inducing CAR target antigen and (ii) to maintain the cell death inducing CAR positive population (percentage or detectable level of cell death inducing CAR immune cell (s) in the total immune cell population by a technique known by the skilled person in the art such as flow cytometry) overtime in the absence of cell death inducing CAR target cells.

According to certain embodiments, the promoter is a promoter that permits to obtain a level of expression sufficient to (i) specifically deplete the cell death inducing CAR immune cells population with target cells presenting the cell death inducing CAR target antigen and (ii) to maintain the cell death inducing CAR positive population (percentage of cell death inducing CAR immune cell in the total immune cell population) overtime in the absence of cell death inducing CAR target cells.

According to certain embodiments, the promoter is a weak promoter. A “weak” promoter is meant to be a promoter having the same or similar promoter activity, i.e. the level of expression of a reporter gene, than the promoter pGK1 when tested under the same experimental conditions.

Methods to study promoter activity are well-known, and include methods based on the detection of expression of a luciferase reporter gene. An exemplary luciferase based assay is the Dual-Luciferase® Reporter Assay System commercially available from Promega (Promega Corporation, Wisconsin, USA).

A suitable promoter may, for example, be one selected from the group consisting of pGK1 (SEQ ID NO: 50), pEF1a short (SEQ ID NO: 55), pUBC (SEQ ID NO: 56), pLCK (SEQ ID NO: 57), pTCF7L1 (SEQ ID NO: 58), pTCF7L2 (SEQ ID NO: 59), pTCF7 (SEQ ID NO: 60) and derivatives of any of the aforesaid.

According to certain embodiments, the nucleic acid sequence encoding the cell death inducing CAR is operably linked to a promote selected from the group consisting of pGK1 (SEQ ID NO: 51), pEF1a short (SEQ ID NO: 55), pUBC (SEQ ID NO: 56), pLCK (SEQ ID NO: 57), pTCF7L1 (SEQ ID NO: 58), pTCF7L2 (SEQ ID NO: 59), pTCF7 (SEQ ID NO: 60) and derivatives of any of the aforesaid.

According to particular embodiments, the nucleic acid sequence encoding the cell death inducing CAR is operably linked to the promoter pGK1 (SEQ ID NO: 50) or a derivative thereof, such as pGK100 (SEQ ID NO: 51), pGK200 (SEQ ID NO: 52), pGK300 (SEQ ID NO: 53) or pGK400 (SEQ ID NO: 54).

Also contemplated is the use of inducible promoters to counter-act the unspecific basal cell death. Non-limiting examples of inducible promoters include steroid-regulated promoters and tetracycline/doxycycline-regulated promoters.

In case the cell death inducing chimeric antigen receptor and/or or activating chimeric antigen receptor is a multi-chain CAR, at least one polynucleotide is provided which comprises two or more nucleic acid sequences encoding the polypeptide chains composing the multi-chain CAR. According to certain embodiments, a composition is provided comprising a first polynucleotide comprising a nucleotide sequence encoding a first polypeptide chain and a second polynucleotide comprising a nucleotide sequence encoding a second polypeptide chain. Optionally, the composition comprises a third polynucleotide comprising a nucleotide sequence encoding a third polypeptide chain.

According to certain embodiments, the different nucleotide sequences can be included in one polynucleotide or vector which comprises a nucleotide sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA. As non-limiting example, in the present invention, 2A peptides have been used to express into the cell the different polypeptides of the multi-chain CAR.

To direct transmembrane polypeptides into the secretory pathway of a host cell, a secretory signal sequence (also known as a leader sequence, prepro sequence or pre sequence) is provided in nucleic acid sequence or vector sequence. The secretory signal sequence may be that of CD8 alpha, or may be derived from another secreted protein (e.g., t-PA) or synthesized de novo. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830).

According to certain embodiments, the signal peptide comprises the amino acid sequence of SEQ ID NO: 61 or 62 or an amino acid sequence which has least 90%, such as at least 95 sequence identity with SEQ ID NO: 61 or 62. Thus, according to certain embodiments, an cell death inducing CAR of the invention further comprising a signal peptide, preferably the signal peptide comprises the amino acid sequence of SEQ ID NO: 61 or 62 or an amino acid sequence which has least 90%, such as at least 95 sequence identity with SEQ ID NO: 61 or 62. Moreover, according to certain embodiments, an activating CAR further comprising a signal peptide, preferably the signal peptide comprises the amino acid sequence of SEQ ID NO: 61 or 62 or an amino acid sequence which has least 90%, such as at least 95 sequence identity with SEQ ID NO: 61 or 62.

Those skilled in the art will recognize that, in view of the degeneracy of the genetic code, considerable sequence variation is possible among these polynucleotide molecules. Preferably, the nucleotide sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

The present invention encompasses any means allowing a reduced cell surface expression of a cell death inducing chimeric antigen receptor, including the use of a weak promoter, competing proteins, signalling peptides etc.

### Methods for Engineering an Immune Cell

The present invention further relates to methods of preparing immune cells for immunotherapy comprising introducing into said immune cells an cell death inducing CAR according to the present invention, optionally together with an activating CAR as detailed herein. In particular, a method for engineering an immune cell is provided, said method comprises:

(i) Providing an immune cell, such as such as T cell; and

(ii) Expressing on the surface of said immune cell at least one cell death inducing chimeric antigen receptor according to the present invention.

According to certain embodiments, the method comprises:

(a) Providing an immune cell;

(b) Introducing into said cell at least one polynucleotide or vector according to the present invention; and

(c) Expressing a cell death inducing chimeric antigen receptor of the invention in said cell.

According to certain embodiments, the method may further comprise

(d) Introducing into said cell at least one polynucleotide or vector encoding an activating chimeric antigen receptor as detailed herein; and

(e) Expressing said activating chimeric antigen receptor in said cell.

In a preferred embodiment, said polynucleotides are included in lentiviral vectors in view of being stably expressed in the cells.

Optionally, the method for engineering an immune cell of the invention may further comprise one or more additional genomic modification steps. Such additional genomic modification step(s) may include modifying said immune cell by editing (e.g., inactivating) at least one gene selected from TCR encoding genes, immune check point genes, genes involved in drug resistance, and combinations thereof.

According to certain embodiments, the genomic modification step includes the inactivation of at least one gene selected from the group consisting of B2M gene, CIITA gene, CD52 gene, GR gene, TCR alpha gene, TCR beta gene, HLA gene, immune check point genes such as PD1 gene and CTLA-4 gene, drug sensitizing genes, such as the dCK gene and HPRT gene, and drug resistance genes. Further details on suitable genes which may be inactivated in accordance with the invention are given further below.

Methods for inactivating genes are known in the art, and include those using rare-cutting endonucleases which able to selectively inactivate by DNA cleavage, preferably by double-strand break, a gene(s) of interest. A gene of interest may be inactivated by transforming the immune cell with a polynucleotide comprising a nucleotide sequence encoding a rare-cutting endonuclease able to selectively inactivate by DNA cleavage, preferably by double-strand break said gene. Said rare-cutting endonuclease can be a meganuclease, a Zinc finger nuclease or a TALE-nuclease. Preferred methods and relevant TALE-nucleases have been described in WO2013/176915. Alternatively, a rare-cutting RNA-guided endonuclease such as Cas9 or DNA-guided endonuclease, such as Argonaute, can be used to inactivate a gene of interest. Suitable techniques are described in, e.g., WO 2013/176915 and WO 2014/191128.

### Delivery Methods

The different methods described above involve introducing a cell death inducing CAR and/or an activating CAR into a cell. As non-limiting example, a CAR can be introduced as transgenes encoded by one plasmidic vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

### Engineered Immune Cells of the Invention

The present invention also relates to engineered immune cells, e.g., isolated engineered immune cells, or engineered immune cell lines.

An “immune cell”, as referred to herein, means a cell of hematopoietic origin functionally involved in the initiation and/or execution of innate and/or adaptative immune response.

An engineered immune cell, e.g. isolated engineered immune cell, according to the present invention comprises (e.g., expresses at its cell surface) at least one cell death inducing CAR of the present invention.

According to certain embodiments, the engineered immune cell further comprises (such as expresses at its cell surface) an activating receptor as detailed herein.

The present invention thus provides an engineered immune cell comprising at least one cell death inducing CAR as described above, in combination with any one of the activating CARs described above.

An engineered immune cell according to the present invention can be derived from a stem cell. A stem cell comprising (e.g., expressing at its cell surface) at least one cell death inducing CAR of the present invention is also contemplate within the scope of the present invention. The stem cell can be a pluripotent or multipotent stem cell. The stem cell can be an adult stem cell, an embryonic stem cell, cord blood stem cell, progenitor cell, bone marrow stem cell, induced pluripotent stem cell, or hematopoietic stem cell.

Representative human cells are CD34+ cells. An immune cell of the present invention can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes, tumor infiltrating lymphocytes or helper T-lymphocytes.

According to certain embodiments, said immune cell can be derived from a CD4+T-lymphocytes or CD8+T-lymphocytes. According to particular embodiments, said immune cell is be derived from CD4+T-lymphocytes.

According to certain embodiments, the engineered immune cell is a human immune cell, such as a human T-lymphocyte.

According to certain embodiments, the engineered immune cell is a primary human immune cell, such as a primary human T-lymphocyte.

“Primary” means isolated from a donor, preferably a healthy donor or a patient in need thereof (i.e. in need of immunotherapy) and maintained in vitro for 1 to 10000, such as 1 to 1000, divisions or maintained living in culture for about 15 days to 25 days in vitro.

To keep immune cells in a proliferation state, avoiding a precocious re-administration of new engineered immune cells, it may be appropriate to use virus-specific T cells (VSTs). Without being cytotoxic in their native form, VSTs are stimulated by endogenous viral antigen by engagement of their native receptors, and then are allowed to proliferate. Expansion and persistence would occur irrespectively of the presence of the CAR target antigen. When engineered according to the present invention, the VSTs may benefit from their properties of proliferation without the presence of the CAR target antigen, while non-VSTs T cells would not proliferate and finally die. Donor-derived virus-specific T cells engineered to express a CD19 specific chimeric antigen receptor and the generation thereof has been described in Cruz et al. (2013, Blood, 122(17): 2965-73).

Thus, according to certain embodiments, the immune cell is a virus-specific T cell (VST), preferably isolated from a donor.

Prior to expansion and genetic modification of the immune cells of the invention, a source of cells can be obtained from a subject, including a human, through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

An engineered immune cell according to the present invention may be modified to inactivate the gene(s) encoding of beta 2-microglobulin (B2M) and/or class II major histocompatibility complex transactivator (CIITA). Thus, according to certain embodiments, the engineered immune cell further comprise at least one inactivated gene selected beta 2-microglobulin (B2M) gene encoding and class II major histocompatibility complex transactivator (CIITA) gene.

An engineered immune cell according to the present invention may further be modified to “less alloreactive”, also called “allogenic” immune cell. Thus, according to certain embodiments, the immune cell further comprise at least one inactivated gene, for example by TALEN®-induced gene knock-out (KO), selected from the group consisting of CD52, GR, TCR alpha (TRAC), TCR beta, HLA gene, beta 2-microglobulin (B2M), immune check point genes such as PD1 and CTLA-4, or can express a pTalpha transgene. More particularly, the immune cell may comprise at least one inactivated gene selected TCR alpha and TCR beta genes. Such inactivation renders the endogenous TCR not functional in the cells and/or reduces, preferably to an undetectable level, cell surface expression of any endogenous TCR. According to certain embodiments, such inactivation inhibits definitively (not temporarily) cell surface expression of TCR in the cells. This strategy is particularly useful to avoid Graft versus Host Disease (GvHD).

According to certain embodiments, the engineered immune cell comprises at least one inactivated gene encoding a component of the T-cell receptor (TCR).

The component of the T-cell receptor may comprise for example a TCR alpha or a TCR beta subunit. A TCR is composed of six different chains that form the TCR heterodimer responsible for ligand recognition. CD3 molecules are assembled together with the TCR heterodimer. CD3 possess a characteristic sequence motif for tyrosine phosphorylation, known as ITAMs (immunoreceptor tyrosine-based activation motifs). The TCR polypeptides themselves have very short cytoplasmic tails, and all proximal signaling events are mediated through the CD3 molecules. TCR-CD3 complex interaction plays an important role in mediating cell recognition events.

Preferably, an engineered immune cell according to the present invention expresses at its cell surface a cell death inducing CAR and an activating CAR, and comprises at least one inactivated gene, for example by TALEN®-induced gene knock-out (KO), selected from the group consisting of CD52, GR, TCR alpha (TRAC), TCR beta, HLA gene, beta2 microglobulin, immune check point genes such as PD1 and CTLA-4, and optionally can express a pTalpha transgene.

An engineered immune cell according to the present invention may further be modified to be resistant to a drug, such as a chemotherapy drug. The term “drug resistance” or “resistance to a drug” refers to the condition when a disease does not respond to the treatment of a drug or drugs. Drug resistance can be either intrinsic (or primary resistance), which means the disease has never been responsive to the drug or drugs, or it can be acquired, which means the disease ceases responding to a drug or drugs that the disease had previously responded to (secondary resistance). In certain embodiments, drug resistance is intrinsic. In certain embodiments, the drug resistance is acquired.

According to certain embodiments, the immune cell further comprises at least one inactivated gene responsible for the cell's sensitivity to the drug (drug sensitizing gene(s)), such as the dCK gene and/or HPRT gene.

According to certain embodiments, the immune cell further comprises an inactivated dCK gene. Preferably, the inactivation of the dCK gene in the immune cell is mediated by a TALE nuclease. To achieve this goal, several pairs of dCK TALE-nuclease have been designed, assembled at the polynucleotide level and validated by sequencing. Examples of TALE-nuclease pairs which can be used according to the invention are depicted in PCT/EP2014/075317. This dCK inactivation in T cells confers resistance to purine nucleoside analogs (PNAs) such as clofarabine and fludarabine.

According to certain embodiments, the dCK inactivation is combined with an inactivation of TRAC genes rendering these double knock out (KO) T cells both resistant to a drug such as clofarabine and less allogeneic. These double features are particularly useful for a therapeutic goal, allowing “off-the-shelf” allogeneic cells for immunotherapy in conjunction with chemotherapy (at least one PNAs) to treat patients with cancer. This double KO inactivation dCK/TRAC can be performed simultaneously or sequentially. One example of TALE-nuclease dCK/TRAC pairs which gave success in the invention is described in PCT/EP2014/075317, in particular, the target sequences in the 2 loci (dCK and TRAC), preferably in 90% of engineered cells.

According to certain embodiments, the immune cell further comprises an inactivated hypoxanthine-guanine phosphoribosyl transferase (HPRT) gene. In particular, HPRT can be inactivated in engineered immune cells to confer resistance to a cytostatic metabolite, the 6-thioguanine (6TG) which is converted by HPRT to cytotoxic thioguanine nucleotide and which is currently used to treat patients with cancer, in particular leukemias (Hacke, Treger et al. 2013). Guanines analogs are metabolized by HPRT transferase that catalyzes addition of phosphoribosyl moiety and enables the formation of TGMP Guanine analogues including 6 mercapthopurine (6MP) and 6 thioguanine (6TG) are usually used as lymphodepleting drugs to treat ALL. They are metabolized by HPRT (hypoxanthine phosphoribosyl transferase that catalyzes addition of phosphoribosyl moiety and enables formation TGMP. Their subsequent phosphorylations lead to the formation of their triphosphorylated forms that are eventually integrated into DNA. Once incorporated into DNA, thio GTP impairs fidelity of DNA replication via its thiolate groupment and generate random points mutation that are highly deleterious for cell integrity.

Alternatively, the drug resistance can be conferred to an immune cell, such as a T cell, by (over)expressing a drug resistance gene. For example, variant alleles of several genes such as dihydrofolate reductase (DHFR), inosine monophosphate dehydrogenase 2 (IMPDH2), calcineurin or methylguanine transferase (MGMT) have been identified to confer drug resistance to an immune cell according to the invention.

One example of a drug resistance gene can also be a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In a particular embodiment, the drug resistance gene according to the present invention can be a nucleic acid sequence encoding a mutant form of human wild type DHFR (GenBank: AAH71996.1) which comprises at least one mutation conferring resistance to an anti-folate treatment, such as methotrexate. In particular embodiment, mutant form of DHFR comprises at least one mutated amino acid at position G15, L22, F31 or F34, preferably at positions L22 or F31 (Schweitzer, Dicker et al. 1990); International application WO94/24277; US patent U.S. Pat. No. 6,642,043). In a particular embodiment, said DHFR mutant form comprises two mutated amino acids at position L22 and F31. Correspondence of amino acid positions described herein is frequently expressed in terms of the positions of the amino acids of the form of wild-type DHFR polypeptide set forth in GenBank: AAH71996.1. According to certain embodiments, the serine residue at position 15 is preferably replaced with a tryptophan residue. In another particular embodiment, the leucine residue at position 22 is preferably replaced with an amino acid which will disrupt binding of the mutant DHFR to antifolates, preferably with uncharged amino acid residues such as phenylalanine or tyrosine. In another particular embodiment, the phenylalanine residue at positions 31 or 34 is preferably replaced with a small hydrophilic amino acid such as alanine, serine or glycine.

As used herein, “antifolate agent” or “folate analogs” refers to a molecule directed to interfere with the folate metabolic pathway at some level. Examples of antifolate agents include, e.g., methotrexate (MTX); aminopterin; trimetrexate (Neutrexin™); edatrexate; N10-propargyl-5,8-dideazafolic acid (CB3717); ZD1694 (Tumodex), 5,8-dideazaisofolic acid (IAHQ); 5,10-dideazatetrahydrofolic acid (DDATHF); 5-deazafolic acid; PT523 (N alpha-(4-amino-4-deoxypteroyl)-N delta-hemiphthaloyl-L-ornithine); 10-ethyl-10-deazaaminopterin (DDATHF, lomatrexol); piritrexim; 10-EDAM; ZD1694; GW1843; Pemetrexate and PDX (10-propargyl-10-deazaaminopterin).

Another example of drug resistance gene can also be a mutant or modified form of ionisine-5′-monophosphate dehydrogenase II (IMPDH2), a rate-limiting enzyme in the de novo synthesis of guanosine nucleotides. The mutant or modified form of IMPDH2 is an IMPDH inhibitor resistance gene. IMPDH inhibitors can be mycophenolic acid (MPA) or its prodrug mycophenolate mofetil (MMF). The mutant IMPDH2 can comprises at least one, preferably two mutations in the MAP binding site of the wild type human IMPDH2 (NP_000875.2) that lead to a significantly increased resistance to IMPDH inhibitor. The mutations are preferably at positions T333 and/or S351 (Yam et al., 2006, Mol Ther 14(2): 236-244; Sangiolo et al., 2007, Gene Ther 14(21): 1549-1554; Jonnalagadda et al., 2013, PLoS One 8(6): e65519). In a particular embodiment, the threonine residue at position 333 is replaced with an isoleucine residue and the serine residue at position 351 is replaced with a tyrosine residue. Correspondence of amino acid positions described herein is frequently expressed in terms of the positions of the amino acids of the form of wild-type human IMPDH2 polypeptide set forth in NP_000875.2.

Another drug resistance gene is the mutant form of calcineurin. Calcineurin (PP2B), an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as IL2. FK506 in complex with FKBP12, or cyclosporine A (CsA) in complex with CyPA block NFAT access to calcineurin's active site, preventing its dephosphorylation and thereby inhibiting T-cell activation (Brewin et al., 2009, Blood 114(23): 4792-4803). The drug resistance gene of the present invention can be a nucleic acid sequence encoding a mutant form of calcineurin resistant to calcineurin inhibitor such as FK506 and/or CsA. In a particular embodiment, said mutant form can comprise at least one mutated amino acid of the wild type calcineurin heterodimer a at positions: V314, Y341, M347, T351, W352, L354, K360, preferably double mutations at positions T351 and L354 or V314 and Y341. In a particular embodiment, the valine residue at position 341 can be replaced with a lysine or an arginine residue, the tyrosine residue at position 341 can be replaced with a phenylalanine residue; the methionine at position 347 can be replaced with the glutamic acid, arginine or tryptophane residue; the threonine at position 351 can be replaced with the glutamic acid residue; the tryptophane residue at position 352 can be replaced with a cysteine, glutamic acid or alanine residue, the serine at position 353 can be replaced with the histidine or asparagines residue, the leucine at position 354 can be replaced with an alanine residue; the lysine at position 360 can be replaced with an alanine or phenylalanine residue of a sequence corresponding to GenBank: ACX34092.1. Correspondence of amino acid positions described herein is frequently expressed in terms of the positions of the amino acids of the form of wild-type human calcineurin heterodimer a polypeptide set forth in (Gen Bank: ACX34092.1).

In another particular embodiment, said mutant form can comprise at least one mutated amino acid of the wild type calcineurin heterodimer b at positions: V120, N123, L124 or K125, preferably double mutations at positions L124 and K125. In a particular embodiment, the valine at position 120 can be replaced with a serine, an aspartic acid, phenylalanine or leucine residue; the asparagine at position 123 can be replaced with a tryptophan, lysine, phenylalanine, arginine, histidine or serine; the leucine at position 124 can be replaced with a threonine residue; the lysine at position 125 can be replaced with an alanine, a glutamic acid, tryptophan, or two residues such as leucine-arginine or isoleucine-glutamic acid can be added after the lysine at position 125 in the amino acid sequence corresponding to GenBank: ACX34095.1. Correspondence of amino acid positions described herein is frequently expressed in terms of the positions of the amino acids of the form of wild-type human calcineurin heterodimer b polypeptide set forth in (GenBank: ACX34095.1).

Another drug resistance gene is 0(6)-methylguanine methyltransferase (MGMT) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, Kurpad et al. 1999). In a particular embodiment, AGT mutant form can comprise a mutated amino acid of the wild type AGT position P140 (UniProtKB: P16455). In a preferred embodiment, said proline at position 140 is replaced with a lysine residue.

Another drug resistance gene can be multidrug resistance protein 1 (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents.

Overexpressing multidrug resistance protein 1 has been described to confer resistance to drugs such as Mitoxantrone (Morrow et al., 2006, Mol. Pharmacol, 69:1499-1505). Thus, drug resistance can be conferred to cells by the expression of nucleic acid sequence that encodes MDR-1 (NP_000918).

Still another way of preparing drug resistant cells is to prepare cells with specific mutation (s) such as mutations at Arg486 and Glu571 in the Human Topoisomerase II gene, to confer resistance to amsacrine (Patel et al., 2000, Mol Pharmacol, 57: 784-791.

Still another way of preparing drug resistant cells is to prepare cells overexpressing microRNA-21 to confer resistance to Daunorubicine (Involvement of miR-21 in resistance to daunorubicin by regulating PTEN expression in the leukaemia K562 cell line (Bai et al., 2011, FEBS Letters, 585(2): 402-408).

In a preferred embodiment, cells bearing such a drug resistance conferring mRNA or protein also comprise an inhibitory mRNA or a gene the expression of which is conditioned, allowing the selective destruction of said drug resistant cells in the presence of said drug or upon administration of said drug.

Drug resistance gene can also confer resistance to cytotoxic antibiotics, and can be ble gene or mcrA gene. Ectopic expression of ble gene or mcrA in an immune cell gives a selective advantage when exposed to the chemotherapeutic agent, respectively the bleomycine or the mitomycin C.

According to certain embodiments, an engineered immune cell according to the present invention expresses at its cell surface a cell death inducing CAR and an activating CAR, comprises at least one inactivated gene, for example by TALEN®-induced gene knock-out (KO), selected from the group consisting of CD52, GR, TCR alpha (TRAC), TCR beta, HLA gene, beta2 microglobulin, immune check point genes such as PD1 and CTLA-4, and optionally can express a pTalpha transgene, and further comprises a gene modification conferring said engineered immune cell resistance to one or more drugs (e.g., to one or more anti-cancer drugs such as PNAs or a FLAG based treatment eg FLAG-IDA, Mito FLAG FLAMSA), such as through inactivation of a dCK gene.

FLAG is an acronym for a chemotherapy regimen used for relapsed and refractory acute myeloid leukemia (AML). The standard FLAG regimen consists of:

1. FLudarabine: an antimetabolite that is not active toward AML, but increases formation of an active cytarabine metabolite, ara-CTP, in AML cells;

2. High-dose cytarabine (Arabinofuranosyl cytidine, or ara-C): an antimetabolite that has been proven to be the most active toward AML among various cytotoxic drugs in single-drug trials;

3. Granulocyte colony-stimulating factor (G-CSF): a glycoprotein that shortens the duration and severity of neutropenia.

FLAG and FLAG-based regimens can also be used in cases of concomitant AML and either acute lymphoblastic leukemia (ALL) or lymphoma. Because fludarabine is highly active in lymphoid malignancies, these regimens can further be used when patients have biphenotypic AML, in which cells display properties of both myeloid and lymphoid cells.

According to certain preferred embodiments, an engineered immune cell, such as a primary T cell, according to the present invention expresses at its cell surface a cell death inducing CAR, which comprises at least one death domain from human CD95 comprising a K296A mutation, and an activating CAR, and comprises an inactivated TCR alpha (TRAC) gene, an inactivated beta2 microglobulin gene and an inactivated dCK or CD52 gene.

According to other certain preferred embodiments, an engineered immune cell, such as a primary T cell, according to the present invention expresses at its cell surface a cell death inducing CAR, which comprises at least one death domain from human CD95 comprising a K296A mutation, and an activating CAR, and comprises at least one inactivated gene, for example by TALEN®-induced gene knock-out (KO), selected from the group consisting of CD52, GR, TCR alpha, TCR beta, HLA gene, beta2 microglubulin, immune check point genes such as PD1 and CTLA-4, and dCK, and optionally can express a pTalpha transgene.

Advantageously, an engineered immune cell, such as a primary T cell, according to the present invention expresses at its cell surface a cell death inducing CAR which comprises at least one death domain as described above, wherein the expression of the cell death inducing CAR is under the control of a promoter as described above. Said cell further comprises an activating CAR, and comprises at least one inactivated gene, for example by TALEV-induced gene knock-out (KO), selected from the group consisting of CD52, GR, TCR alpha, TCR beta, HLA gene, beta2 microglobulin, immune check point genes such as PD1 and CTLA-4, and dCK, and optionally can express a pTalpha transgene.

According to preferred embodiments, an engineered primary immune T cell comprises a cell death inducing CAR comprising a K296A mutation in its intracellular death domain, and an activating CAR, said cells further comprising a TCR alpha, a TCR beta, a beta2 microglobulin and/or a drug resistant knock-out (KO) gene.

Advantageously, an engineered primary T immune cell according to the present invention expresses at its cell surface a cell death inducing CAR which comprises at least one death domain as described above, optionally mutated, wherein the expression of the cell death inducing CAR is under the control of a promoter as described above. Said cell further comprises an activating CAR, and comprises at least one inactivated gene, for example by TALEN®-induced gene knock-out (KO), selected from the group consisting of CD52, GR, TCR alpha, TCR beta, HLA gene, beta2 microglubulin, immune check point genes such as PD1 and CTLA-4, and dCK, and optionally can express a pTalpha transgene.

More advantageously, an engineered primary T immune cell according to the present invention expresses at its cell surface a cell death inducing CAR which comprises at least one death domain as described above, optionally mutated, wherein the expression of the cell death inducing CAR is under the control of a promoter selected from the group consisting of pEF1a short, pGK1, pGK100, pGK200, pGK300 and pGK400. Said cell further comprises an activating CAR, and comprises at least one inactivated gene, for example by TALEN®-induced gene knock-out (KO), selected from the group consisting of CD52, GR, TCR alpha, TCR beta, HLA gene, beta2 microglobulin, immune check point genes such as PD1 and CTLA-4, and dCK KO gene, and optionally comprises a pTalpha transgene.

According to certain embodiments, the engineered immune cell further comprising a polynucleotide comprising a nucleic acid sequence encoding an activating CAR operably linked to a promoter, such as native promoter.

According to certain embodiments, the engineered immune cell further comprising a polynucleotide comprising a nucleic acid sequence encoding an activating CAR operably linked to a promoter selected from pGK1, pUBC, pLCK, pEF1a short, pTCF7L1, pTCF7L2 and pTCF7.

According to certain embodiments, the engineered immune cell further comprising a polynucleotide comprising a nucleic acid sequence encoding an activating CAR operably linked to a promoter selected from pGK1, pUBC, pLCK, pEF1a short, pTCF7L1, pTCF7L2 and pTCF7, inserted into a TCR gene.

### Activation and Expansion of Engineered Immune Cells

Whether prior to or after genetic modification of the immune cells, even if the genetically modified immune cells of the present invention are activated and proliferate independently of antigen binding mechanisms, the immune cells, particularly T-cells of the present invention can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the immune cells of the invention are expanded by contact with an agent that stimulates a CD3 TCR complex and a co-stimulatory molecule on the surface of the T cells to create an activation signal for the T-cell.

For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, −10, −2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). T cells that have been exposed to varied stimulation times may exhibit different characteristics

According to certain embodiments, said cells can be expanded by co-culturing with tissue or cells. Said cells can also be expanded in vivo, for example in the subject's blood after administrating said cell into the subject.

### Therapeutic Applications

Immune cells according to the present invention are intended to be used as a medicament, and in particular for treating cancer in a patient (e.g. a human patient) in need thereof. Accordingly, the present invention provides engineered immune cells for use as a medicament. Particularly, the present invention provides engineered immune cells for use in the treatment of a cancer. Also provided are compositions, particularly pharmaceutical compositions, which comprise at least one engineered immune cell of the present invention and, optionally, a pharmaceutically acceptable vehicle. In certain embodiments, a composition may comprise a population of immune cells of the present invention.

The treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor and are engineered to be less alloreactive (KO TCR) and produce no or reduced GVHD as compared to cells with intact TRAC gene.

The invention is particularly suited for allogenic immunotherapy, insofar as it enables the transformation of immune cells, such as T-cells, typically obtained from donors, into non-alloreactive cells. This may be done under standard protocols and reproduced as many times as needed. The resultant modified immune cells may be pooled and administrated to one or several patients, being made available as an “off the shelf” therapeutic product.

The treatments are primarily to treat patients diagnosed with cancer. Particular cancers to be treated according to the invention are those which have solid tumors, but may also concern liquid tumors. Adult tumors/cancers and pediatric tumors/cancers are also included.

According to certain embodiments, the engineered immune cell(s), population or composition is for use in the treatment of a cancer, and more particularly for use in the treatment of a solid or liquid tumor. According to certain embodiments, the engineered immune cell(s), population or composition is for use in the treatment of a solid tumor. According to certain embodiments, the engineered immune cell(s), population or composition is for use in the treatment of a liquid tumor.

According to certain embodiments, the engineered immune cell(s), population or composition is for use in the treatment of a cancer selected from the group consisting of lung cancer, small lung cancer, breast cancer, uterine cancer, prostate cancer, kidney cancer, colon cancer, liver cancer, pancreatic cancer, and skin cancer. According certain embodiments, the engineered immune cell(s), population or composition is for use in the treatment of lung cancer. According to certain embodiments, the engineered immune cell(s), population or composition is for use in the treatment of small lung cancer. According to certain embodiments, the engineered immune cell(s), population or composition is for use in the treatment of breast cancer. According to certain embodiments, the engineered immune cell(s) or composition is for use in the treatment of uterine cancer. According to certain embodiments, the engineered immune cell(s), population or composition is for use in the treatment of prostate cancer. According to certain embodiments, the engineered immune cell(s), population or composition is for use in the treatment of kidney cancer. According to certain embodiments, the engineered immune cell(s), population or composition is for use in the treatment of colon cancer. According to certain embodiments, the engineered immune cell(s), population or composition is for use in the treatment of liver cancer. According to certain embodiments, the engineered immune cell(s), population or composition is for use in the treatment of pancreatic cancer. According to certain embodiments, the engineered immune cell(s), population or composition is for use in the treatment of skin cancer.

According to certain embodiments, the immune cell(s), population or composition is for use in the treatment of a sarcoma.

According to certain embodiments, the immune cell(s), population or composition is for use in the treatment of a carcinoma. According to certain embodiments, the engineered immune cell, population or composition is for use in the treatment of renal, lung or colon carcinoma.

According to certain embodiments, the engineered immune cell(s), population or composition is for use in the treatment of leukemia, such as acute lymphoblastic leukemia (ALL), acute myeloid leukemia (AML), chronic lymphocytic leukemia (CLL), chronic myelogenous leukemia (CML), and chronic myelomonocystic leukemia (CMML). According to certain embodiments, the engineered immune cell(s), population or composition is for use in the treatment of acute lymphoblastic leukemia (ALL). According to certain embodiments, the immune cell(s), population or composition is for use in the treatment of acute myeloid leukemia (AML). According to certain embodiments, the engineered immune cell(s), population or composition is for use in the treatment of chronic lymphocytic leukemia (CLL). According to certain embodiments, the immune cell(s), population or composition is for use in the treatment of chronic myelogenous leukemia (CML). According to certain embodiments, the immune cell(s) or composition is for use in the treatment of chronic myelomonocystic leukemia (CMML).

According to certain embodiments, the immune cell(s), population or composition is for use in the treatment of lymphoma, such as B-cell lymphoma. According to certain embodiments, the engineered immune cell(s), population or composition is for use in the treatment of primary CNS lymphoma. According to certain embodiments, the engineered immune cell(s), population or composition is for use in the treatment of Hodgkin's lymphoma. According to certain embodiments, the engineered immune cell(s), population or composition is for use in the treatment of Non-Hodgkin's lymphoma. According to certain embodiments, the engineered immune cell(s), population or composition is for use in the treatment of diffuse large B cell lymphoma (DLBCL). According to certain embodiments, the engineered immune cell(s), population or composition is for use in the treatment of Follicular lymphoma. According to certain embodiments, the engineered immune cell(s), population or composition is for use in the treatment of marginal zone lymphoma (MZL). According to certain embodiments, the engineered immune cell(s), population or composition is for use in the treatment of Mucosa-Associated Lymphatic Tissue lymphoma (MALT). According to certain embodiments, the engineered immune cell(s), population or composition is for use in the treatment of small cell lymphocytic lymphoma. According to certain embodiments, the engineered immune cell(s), population or composition is for use in the treatment of mantle cell lymphoma (MCL). According to certain embodiments, the engineered immune cell(s), population or composition is for use in the treatment of Burkitt lymphoma. According to certain embodiments, the engineered immune cell(s), population or composition is for use in the treatment of primary mediastinal (thymic) large B-cell lymphoma. According to certain embodiments, the engineered immune cell(s), population or composition is for use in the treatment of Waldenström macroglobulinemia. According to certain embodiments, the engineered immune cell(s), population or composition is for use in the treatment of nodal marginal zone B cell lymphoma (NMZL). According to certain embodiments, the engineered immune cell(s), population or composition is for use in the treatment of splenic marginal zone lymphoma (SMZL). According to certain embodiments, the engineered immune cell(s), population or composition is for use in the treatment of intravascular large B-cell lymphoma. According to certain embodiments, the engineered immune cell(s), population or composition is for use in the treatment of Primary effusion lymphoma. According to certain embodiments, the engineered immune cell(s), population or composition is for use in the treatment of lymphomatoid granulomatosis. According to certain embodiments, the engineered immune cell(s), population or composition is for use in the treatment of T cell/histiocyte-rich large B-cell lymphoma. According to certain embodiments, the engineered immune cell(s), population or composition is for use in the treatment of primary diffuse large B-cell lymphoma of the CNS (Central Nervous System). According to certain embodiments, the engineered immune cell(s), population or composition is for use in the treatment of primary cutaneous diffuse large B-cell lymphoma. According to certain embodiments, the engineered immune cell(s), population or composition is for use in the treatment of EBV positive diffuse large B-cell lymphoma of the elderly. According to certain embodiments, the engineered immune cell(s) population or composition is for use in the treatment of diffuse large B-cell lymphoma associated with inflammation. According to certain embodiments, the engineered immune cell(s), population or composition is for use in the treatment of ALK-positive large B-cell lymphoma. According to certain embodiments, the engineered immune cell(s), population or composition is for use in the treatment of plasmablastic lymphoma. According to certain embodiments, the engineered immune cell(s), population or composition is for use in the treatment of Large B-cell lymphoma arising in HHV8-associated multicentric Castleman disease.

According to certain embodiments, the engineered immune cell(s), population or composition is for use in the treatment of a viral infection, such as an HIV infection or HBV infection.

According to certain embodiments, the engineered immune cell(s), population or composition is for use as vaccine. The destruction of immune cells by apoptosis or necrosis of immune cells near a tumor cell will produce a local microvaccination that contributes to the presentation of novel tumor antigen(s) and overall reduction of tumoral mass.

According to certain embodiment, the immune cell originates from a patient, e.g. a human patient, to be treated. According to certain other embodiment, the immune cell originates from at least one donor.

The treatment can take place in combination with one or more therapies selected from the group of antibodies therapy, chemotherapy, cytokines therapy, anti-cytokine therapy, dendritic cell therapy, gene therapy, hormone therapy, laser light therapy and radiation therapy.

According to certain embodiments, engineered immune cells of the invention can undergo robust in vivo immune cell expansion upon administration to a patient, and can persist in the body fluids for an extended amount of time, preferably for a week, more preferably for 2 weeks, even more preferably for at least one month. Although the immune cells according to the invention are expected to persist during these periods, their life span into the patient's body are intended not to exceed a year, preferably 6 months, more preferably 2 months, and even more preferably one month.

The administration of the engineered immune cell(s), population or composition according to the present invention may be carried out in any convenient manner, including by aerosol inhalation, injection, ingestion, transfusion, implantation or transplantation. The immune cells or composition described herein may be administered to a patient subcutaneously, intradermally, intratumorally, intranodally, intramedullary, intramuscularly, by intravenous or intralymphatic injection, or intraperitoneally.

According to certain embodiments, the engineered immune cell(s), population or composition is administered by intravenous injection.

According to certain embodiments, the engineered immune cell(s), population or composition is administrated parenterally.

According to certain embodiments, the engineered immune cell(s), population or composition is administered intratumorally. Said administration can be done by injection directly into a tumor or adjacent thereto.

The administration of the cells or population of cells can consist of the administration of 10⁴-10⁹ cells per kg body weight, preferably 10⁵ to 10⁶ cells/kg body weight including all integer values of cell numbers within those ranges. The cells or population can be administrated in one or more doses. According to certain embodiments, an effective amount of cells is administrated as a single dose. According to certain embodiments, an effective amount of cells is administrated as more than one dose over a period time. Timing of administration is within the judgment of managing physician and depends on the clinical condition of the patient. The cells or population may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

According to certain embodiments, the engineered immune cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p7056 kinase that is important for growth factor induced signaling (rapamycin) (Liu et al., Cell 66:807-815, 1 1; Henderson et al., Immun. 73:316-321, 1991; Bierer et al., Citrr. Opin. mm n. 5:763-773, 93). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, According to certain embodiments, the engineered immune cells are administered following B-cell ablative therapy such as agents that react with CD20, e.g., Rituxan. For example, in one embodiment, subjects may undergo standard treatment with high dose chemotherapy followed by peripheral blood stem cell transplantation. According to certain embodiments, following the transplant, subjects receive an infusion of the expanded engineered immune cells of the present invention. In an additional embodiment, expanded cells are administered before or following surgery.

Also encompassed within this aspect of the invention are methods for treating a patient in need thereof, comprising a) providing at least one immune cell of the present invention, preferably a population of said immune cell; and b) administering said immune cell or population to said patient.

Also encompassed within this aspect of the invention are methods for preparing a medicament comprising at least one engineered immune cell of the present invention, and preferably a population of said immune cell. Accordingly, the present invention provides the use of at least one engineered immune cell of the present invention, and preferably a population of said immune cell, in the manufacture of a medicament. Preferably, such medicament is for use in the treatment of a disease as specified above.

Accordingly, the present invention provides at least one engineered immune cell of the present invention, and preferably a population of said immune cell, for use in the manufacture of a medicament. Preferably, such medicament is for the treatment of a disease as specified above.

### Other Definitions

“ectodomain” refers to a part of a chimeric antigen receptor of the present invention which extends into the extracellular space (the space outside a cell).
“endodomain” refers to a part of a chimeric antigen receptor of the present invention which extends into the cytoplasm of a cell.
“self-association” is intended to mean selective intermolecular interactions of two or more domains of the same identity that are not driven by an external stimulus (e.g. ligand binding).
By “apoptosis” is meant a process of programmed and targeted cell death that occurs in multicellular organisms. Biochemical events that characterized apoptosis lead to characteristic cell changes (morphology) and death. These changes include blebbing, cell shrinkage, nuclear fragmentation, chromatin condensation, chromosomal DNA fragmentation, and global mRNA decay.
By “Necrosis” is meant a form of cell injury which results in the premature non programmed death of cells in living tissue by autolysis.
Amino acid residues in a polypeptide sequence are designated herein according to the one-letter code, in which, for example, Q means Gln or Glutamine residue, R means Arg or Arginine residue and D means Asp or Aspartic acid residue.
By “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
“Substitution” or “substituted” refers to a modification of a polypeptide by replacing one amino acid residue with another. For instance the replacement of an arginine residue with an alanine residue in a polypeptide sequence is an amino acid substitution.
“Conservative substitution” refers to a substitution of an amino acid residue with a different residue having a similar side chain, and thus typically involves substitution of the amino acid in the polypeptide with amino acids within the same or similar class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having an aromatic side chain is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.
“Non-conservative substitution” refers to substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid. Preferably, the non-conservative amino acid substitution includes substituting a given amino acid by an aliphatic amino acid, such as glycine or alanine.
“Deletion” or “deleted” refers to modification of a polypeptide by removal of one or more amino acids in the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining polypeptide function. Deletions can be directed to the internal portions and/or terminal portions of the polypeptide, in various embodiments, the deletion can comprise a continuous segment or can be discontinuous.
“Insertion” or “inserted” refers to modification of the polypeptide by addition of one or more amino acids to the reference polypeptide. Insertions can comprise addition of 1 or more amino acids, 2 or more amino acids, 5 or more amino acids, 10 or more amino acids, 15 or more amino acids, or 20 or more amino acids. Insertions can be in the internal portions of the polypeptide, or to the carboxy or amino terminus. The insertion can be a contiguous segment of amino acids or separated by one or more of the amino acids in the reference polypeptide.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.
As used herein, the term “locus” is the specific physical location of a DNA sequence (e.g. of a gene) on a chromosome. The term “locus” can refer to the specific physical location of a rare-cutting endonuclease target sequence on a chromosome. Such a locus can comprise a target sequence that is recognized and/or cleaved by a rare-cutting endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.
“Inactivating” or “inactivation or a gene” means that the gene of interest is not expressed in a functional protein form. Methods for inactivating genes are known in the art, such as the use of rare-cutting endonucleases which are able to selectively inactivate by DNA cleavage, preferably by double-strand break, the gene(s) of interest. “Inactivating” or “inactivation” also includes deletion of a part of or the entire gene sequence, such as by gene replacement.

The presence or absence of a gene on the chromosome of a given cell can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including SDS-PAGE followed by an immunoblotting assay (Western blotting analysis), and the like.
“operably linked” refers to two nucleic acid sequences that are related physically or functionally. For example, a regulatory element, such as a promoter, is said to be “operably linked” to a coding sequence if the two sequences are situated such that the regulatory DNA sequence affects expression of the coding DNA sequence. Coding sequences may be operably linked to regulatory sequences in sense or antisense orientation.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, or penetrating peptides. In these later cases, delivery vectors are molecule carriers.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adeno-associated viruses), coronavirus, negative strand RNA viruses such as orthomyxovirus (e. g., influenza virus), rhabdovirus (e. g., rabies and vesicular stomatitis virus), para-myxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomega-lovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non limiting example, that are able to integrate the genome of a target cell. At the opposite by “non integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
By “cell” or “cells” is intended any eukaryotic living cells, primary cells and cell lines derived from these organisms for in vitro cultures.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.
By “cell line” is intended an isolated cell population of cell preferably an isolated primary engineered cell, expanded and purified to comprise at least 80%, 95% preferably 99% more preferably 99.90% of one cell type preferably cells are non transformed cells.

As non-limiting examples cell lines (of transformed cell lines) can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.
By “stem cell” is meant a cell that has the capacity to self-renew and the ability to generate differentiated cells. More explicitly, a stem cell is a cell which can generate daughter cells identical to their mother cell (self-renewal) and can produce progeny with more restricted potential (differentiated cells).
By “NK cells” is meant natural killer cells. NK cells are defined as large granular lymphocytes and constitute the third kind of cells differentiated from the common lymphoid progenitor generating B and T lymphocytes.
By “variant(s)”, it is intended a polypeptide variant obtained by mutation or replacement of at least one residue in the amino acid sequence of the parent molecule.
By “fusion protein” is intended the result of a well-known process in the art consisting in the joining of two or more genes which originally encode for separate proteins or part of them, the translation of said “fusion gene” resulting in a single polypeptide with functional properties derived from each of the original proteins.
“identity”, “percentage of sequence identity,” “% sequence identity” and “percent identity” are used herein to refer to comparisons between an amino acid sequence and a reference amino acid sequence. The “% sequence identify”, as used herein, is calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence. For example, polypeptides having at least 80%, 85%, 90%, 95%, 98% or 99% identity to specific polypeptides described herein and preferably exhibiting substantially the same functions, as well as polynucleotide encoding such polypeptides, are contemplated.
“Reference sequence” or “reference amino acid sequence” refers to a defined sequence to which another sequence is compared.
The term “subject” or “patient” as used herein includes all members of the animal kingdom including non-human primates and humans.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

### Example 1. Cell Death Inducing CARs

Cell death inducing CARs are designed to be composed of signal peptide for targeting to the cell surface (SEQ ID: 63), an antigen targeting domain (SEQ ID: 64 to 65) followed by a stalk (or hinge) domain (SEQ ID: 66 to 67), a transmembrane domain (SEQ ID: 68) and an intracellular domain (SEQ ID: 69). The intracellular domain containing a so-called death domain is intended to promote cell death upon engagement of the antigen recognition domain. Cell death inducing CARs are cloned into lentiviral production plasmids (genome plasmid) upstream of a 2A (SEQ ID: 70) cis-acting hydrolase element followed by a reporter marker (e.g. fluorescent proteins SEQ ID: 71 to 72) under the control of different promoters (SEQ ID: 73 to 75). Standard molecular biology technics such as PCR (Agilent Herculase II fusion Enzyme cat#600677), enzymatic restriction digestions (New England Biolabs or ThermoFisher), ligations (T4 DNA ligase cat#EL0011) and bacterial transformations (XL1b, Agilent cat#200236 or One shot Stbl3, ThermoFisher cat#C7373-03) are applied according to the manufacturer instructions to create cell death inducing CARs.

The intracellular and transmembrane domain from human CD95 (SEQ ID NO: 69 and 68, respectively) was used in combination with either a CD8a-based (SEQ. ID: 66) or IgG1-based (SEQ ID NO: 67) hinge and a CD19 targeting scFv (SEQ. ID: 65), leading D-CAR1 (SEQ ID NO: 76) and D-CAR2 (SEQ ID NO: 78), respectively.

The integrity of the cell death inducing CAR sequences is verified by Sanger DNA sequencing (GenScript). Genome plasmids used for lentiviral particules preparation are obtained from One shot Stb13 transformation and purified using Nucleobond Maxi Xtra EF kits (Macherey-Nagel cat#740424.50). Lentiviral particles are generated in 293FT cells (ThermoFisher) cultured in RPMI 1640 Medium (ThermoFisher cat#SH30027FS) supplemented with 10% FBS (Gibco cat#10091-148), 1% HEPES (Gibco cat#15630-80), 1% L-Glutamine (Gibco cat#35050-61) and 1% Penicilin/Streptomycin (Gibco cat#15070-063) using Opti-MEM medium (Gibco cat#31985-062) and Lipofectamine 2000 (Thermo Fisher cat#11668-019) according to standard transfection procedures. 48 and/or 72 hours post transfection the supernatants are recovered and concentrated by ultracentrifugation.

### Example 2. Characterization of Cell Death Inducing CARs in Immortalized Human T-Cells

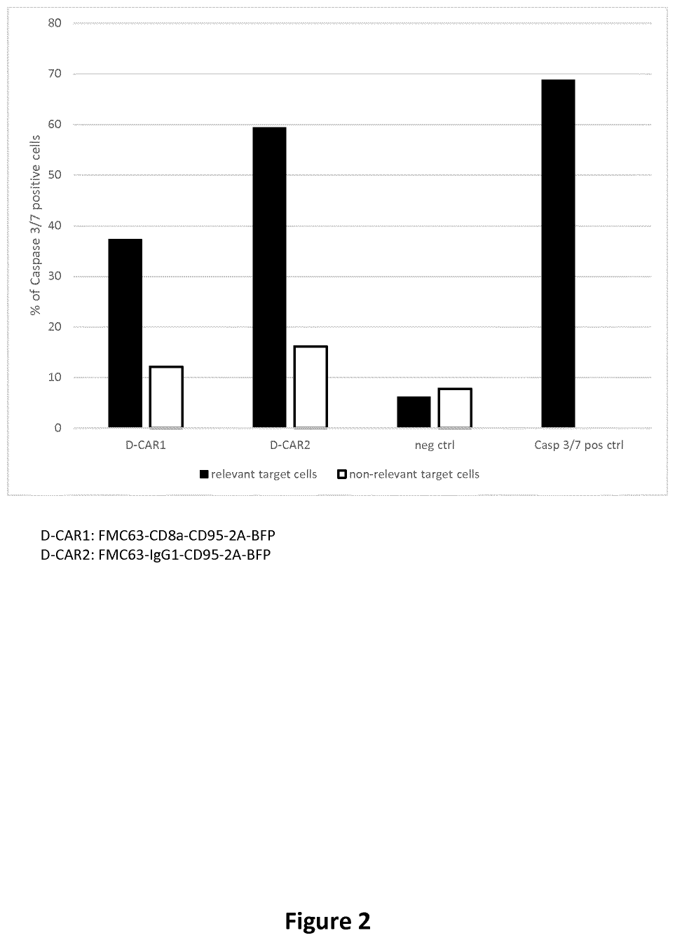

Human T-cell line (Jurkat) are incubated in an untreated 12 well plate pre-coated with 30 μg/mL retronectine (Takara cat#T100B) in the presence of lentiviral particles encoding an cell death inducing CAR (SEQ ID: 76 or 78) in RPMI-1640 serum free medium (ThermoFisher cat# SH30027FS) for 2-3h at 37° C. Twice the volume of growth media containing 20% FBS and 50× dilution of penicillin-streptomycin is added to the cells for overnight incubation. The cells are then washed and cultured in RPMI 1640 Medium (ThermoFisher cat#SH30027FS) for several days supplemented with 10% FBS. The whole bulk cell death CAR population is assessed for Caspase 3/7 activation by co-incubation with model cell lines expressing either the cell death inducing CAR target antigen (CD19 expressing HEK293) or a non-relevant antigen (PSMA expressing HEK293). Caspase 3/7 activation is detected using the CellEvent Caspase-3/7 Green Flow Cytometry Assay Kit (ThermoFisher #catC10427) according to the manufacturer recommendations. The results showed an increase of Caspase 3/7 positive cells only when co-incubation is done with target cells expressing the relevant antigen for D-CAR1 (SEQ ID NO: 76) and D-CAR2 (SEQ ID: 78) (FIG. 2).

### Example 3. Generation of Mutated Cell Death Inducing CARs

Mutations that attenuate the engineered cell death inducing CAR self-association or binding to FADD are incorporated into the cell death inducing CAR constructs using the QuikChange Lightning Multi Site-Directed Mutagenesis Kit (Agilent cat#210514-5). One oligonucleotide containing the desired mutation(s) is designed for each identified position according to the QuikChange Lightning Multi Site-Directed Mutagenesis Kit recommendations and sythetized de novo (IDT, Integrated DNA Technologies). The QuikChange Lightning Multi Site-Directed Mutagenesis procedure is then applied, according to the manufacturer instructions, for each individual oligonucleotide encoding a mutation using a template plasmid encoding the D-CAR2 insert (SEQ ID: 78) previously subcloned in a pSelect backbone (Invivogen). Using the described strategy and oligoK296A (gtatgacacattgattGCagatctcaaaaaagcc; SEQ ID NO: 99) a D-CAR containing the K296A mutation (numbering according to the Uniprot database) is constructed (position K296 in the full length amino acid sequence of Fas corresponds to position K67 in SEQ ID NO: 6).

The integrity of the cell death inducing CAR sequences is verified by Sanger DNA sequencing (GenScript). Standard molecular biology techniques such as PCR (Agilent Herculase II fusion Enzyme cat#600677), enzymatic restriction digestions (New England Biolabs or ThermoFisher), ligations (T4 DNA ligase cat#EL0011) and bacterial transformations (XL1b or One shot Stbl3, ThermoFisher cat#C7373-03) are then applied to subclone the mutated cell death inducing CAR insert into a lentiviral genome plasmid leading to D-CAR3 (SEQ ID NO: 82). The integrity of the cell death inducing CAR sequences is verified by Sanger DNA sequencing (GenScript).

### Characterization of Cell Death Inducing CARs and Mutated Cell Death Inducing CARs in Primary T-Cells

PBMCs are thawed and plated at 1×10⁶ cells/ml media in X-vivo-15 media (Lonza cat#BE04-418Q) supplemented with 5% AB serum (Seralab cat#GEM-100-318) and 20 ng/ml IL-2 (Miltenyi Biotech cat#130-097-748) for overnight culture at 37° C.

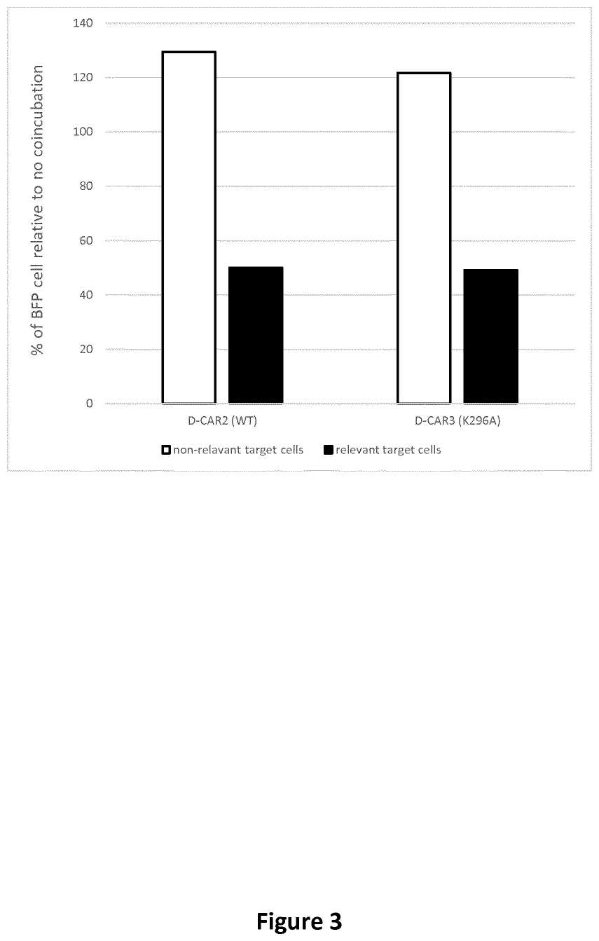

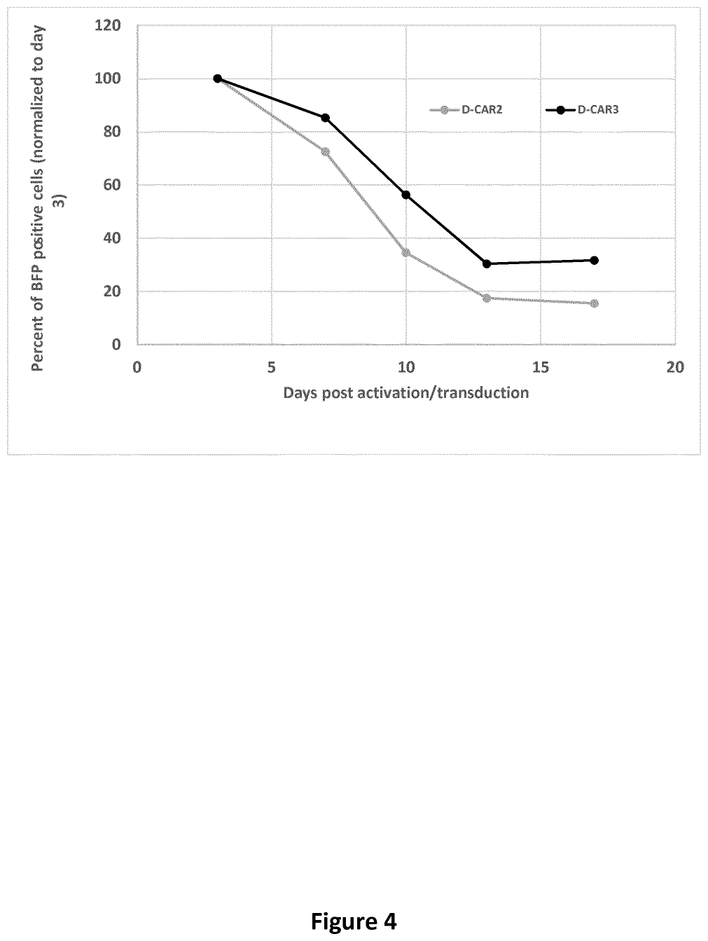

PBMCs are activated using human T activator CD3/CD28 (Life Technology cat#11132D) in X-vivo-15 media supplemented with 5% AB serum and 20 ng/ml IL-2. 1×10⁶ activated PBMCs (in 600 μl) are immediately incubated without removing the beads in an untreated 12 well plate pre-coated with 30 μg/mL retronectin (Takara cat#T100B) in the presence of lentiviral particles encoding an cell death inducing CAR (SEQ ID: 78 or 82) for 2h at 37° C. 600 μl of 2× X-vivo-15 media (X-vivo-15, 10% AB serum and 40 ng/ml IL-2) is then added and the cells are incubated at 37° C. for 72h. 3-5 days post transduction T-cells are incubated in complete X-vivo-15 media in a 1:1 ratio with target cells presenting the cell death inducing CAR target antigen (CD19 expressing HEK293) or an irrelevant antigen (PSMA expressing HEK293) for up to 24 hours. The whole bulk cell population (T-cells and target cell) is then assessed for cell death inducing CAR expression by measuring the percentage of viable BFP reporter positive cells by flow cytometry. Target cells are excluded from the analysis by either gating exclusively on CD3 positive cells or by pre-labeling target cells with CellTrace Far Red (ThermoFisher cat# C34572). The percentage of EGFP positive cell is then normalized to the one measured in absence of target cells. The results showed a decrease of BFP positive cells only when co-incubation is done with target cells expressing the relevant antigen for D-CAR2 (SEQ ID: 78) or D-CAR3 (SEQ ID NO: 82) (FIG. 3). Furthermore, the reporter positive population is followed overtime with a reactivation with CD8/CD28 beads at day 14. The results showed that the BFP positive cell population is better maintained overtime with the D-CAR3 when compared to D-CAR2, suggesting a higher viability of the immune cells endowed with the mutant cell death inducing CAR (FIG. 4).

### Example 4

Cell death inducing CARs based on DR4 and DR5 are constructed according to standard molecular biology techniques, as described in example 1, using an scFv targeting PSMA (SEQ ID: 90), their native hinge domain (amino acid sequence from the transmembrane domain up to the first annotated extracellular topological domain) (SEQ ID: 91 to 92), their native transmembrane domains (SEQ ID: 93 to 94) and their endoplasmic domains (SEQ ID: 95 to 96) and EGFP as reporter (SEQ ID: 71), leading to SEQ ID: 97 to 98. Lentiviral particles are obtained as described in example 1.

### Characterization of DR4 and DR5 Based Cell Death Inducing CARs in Primary T-Cells

PBMCs are thawed and plated at 1×10⁶ cells/ml media in X-vivo-15 media (Lonza cat#BE04-418Q) supplemented with 5% AB serum (Seralab cat#GEM-100-318) and 20 ng/ml IL-2 (Miltenyi Biotech cat#130-097-748) for overnight culture at 37° C. PBMCs are activated using human T activator CD3/CD28 (Life Technology cat#11132D) in X-vivo-15 media supplemented with 5% AB serum and 20 ng/ml IL-2. 1×10⁶ activated PBMCs (in 600 μl) are immediately incubated without removing the beads in an untreated 12 well plate pre-coated with 30 μg/mL retronectin (Takara cat#T100B) in the presence of lentiviral particules encoding an cell death inducing CAR (SEQ ID: 97 to 98) for 2h at 37° C. 600 μl of 2× X-vivo-15 media (X-vivo-15, 10% AB serum and 40 ng/ml IL-2) is then added and the cells are incubated at 37° C. for 72h. 3-5 days post transduction T-cells are incubated in complete X-vivo-15 media in a 1:1 ratio with target cells presenting the cell death inducing CAR target antigen (PSMA expressing HEK293) or an irrelevant antigen (CD19 expressing HEK293) for up to 24 hours. The whole bulk cell population (T-cells and target cell) is then assessed for cell death inducing CAR expression by measuring the percentage of viable BFP reporter positive cells by flow cytometry. Target cells are excluded from the analysis by either gating exclusively on CD3 positive cells or by pre-labeling target cells with CellTrace Far Red (ThermoFisher cat# C34572). The percentage of EGFP positive cell is then normalized to the one measured in absence of target cells.

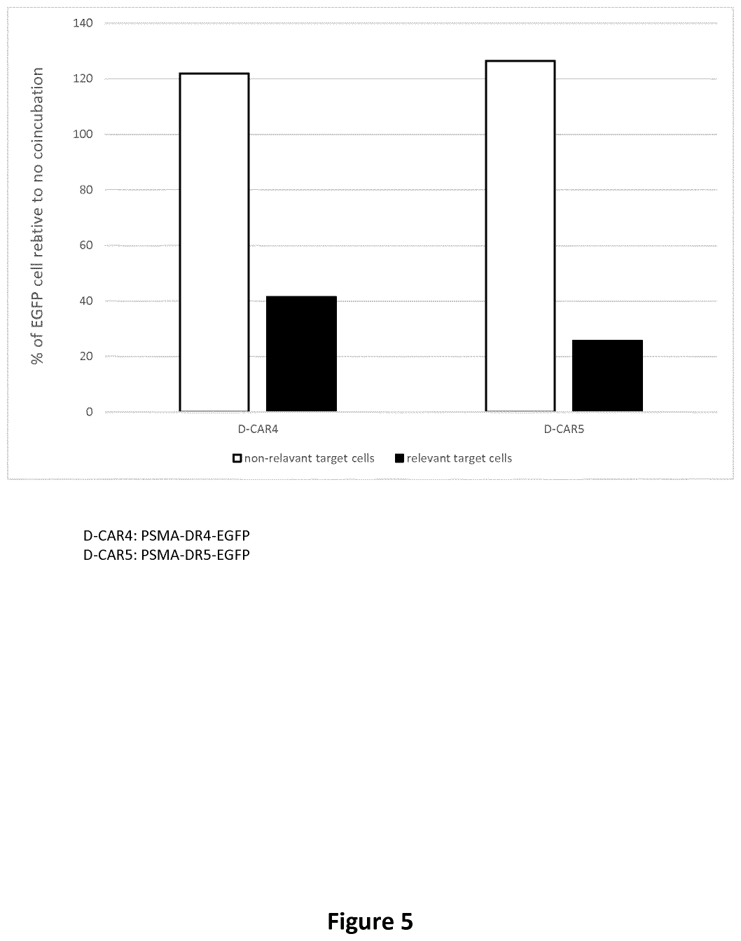

The results showed a decrease of BFP positive cells only when co-incubation is done with target cells expressing the relevant antigen for D-CAR4 and D-CAR5 (SEQ ID: 97 to 98) (FIG. 5).

### Example 5. Insertion of a D-CAR at the TRAC Locus with Knock-Out of the TRAC Endogenous Gene

### A: Expression Controlled by the Native Promoter

The location of the TALEN target site is designed to be located in the exon 2 of the TCRα locus. The sequence encompassing 1000 bp upstream and downstream the TALEN targets is given in SEQ ID: 100 and 101. Target sequences of the TALEN (SEQ ID: 102 and 103) is given in SEQ ID: 104. The integration matrix is designed to be composed of a sequence (1000 bp) homologous to the endogenous gene upstream of the TALEN site (SEQ ID: 100), followed by a 2A regulatory element (SEQ ID: 105), followed by a sequence encoding a D-CAR without the start codon (SEQ ID: 106 and 107), followed by a STOP codon (tag), followed by a polyadenylation sequence (SEQ ID: 108), followed by a sequence (1000 bp) homologous to the endogenous gene downstream of the TALEN site (SEQ ID: 101). The insertion matrix is subsequently cloned into a promoterless rAAV vector and used to produce AAV6.

### B: Expression Controlled by a Specific Promoter According to the Present Invention.

The location of the TALEN target site is designed to be located in the exon 2 of the TCRα locus. The sequence encompassing 1000 bp upstream and downstream the TALEN targets is given in SEQ ID: 100 and 101. Target sequences of the TALEN (SEQ ID: 102 and 103) is given in SEQ ID: 104. The integration matrix is designed to be composed of a sequence (1000 bp) homologous to the endogenous gene upstream of the TALEN site (SEQ ID: 100), followed by a specific promoter, pGK1 (SEQ ID NO: 50) or pEF1a short (SEQ ID NO: 55) or pGK100 (SEQ ID NO: 51) or pGK200 (SEQ ID NO: 52) or pGK300 (SEQ ID NO: 53) or pGK400 (SEQ ID NO: 54), followed by a 5′UTR (SEQ ID NO: 109), followed by a sequence encoding a D-CAR2 with the start codon (SEQ ID: 110) or a D-CAR3 with the start codon (SEQ ID NO: 111), followed by a polyadenylation sequence (SEQ ID: AJ), followed by a sequence (1000 bp) homologous to the endogenous gene downstream of the TALEN site (SEQ ID: 101). The insertion matrix is subsequently cloned into a promoterless rAAV vector and used to produce AAV6.

### General Protocol

TALEN® mRNA is synthesized using the mMessage mMachine T7 Ultra kit (Thermo Fisher Scientific, Grand Island, N.Y.) as each TALEN is cloned downstream of a T7 promoter, purified using RNeasy columns (Qiagen, Valencia, Calif.) and eluted in “cytoporation medium T” (Harvard Apparatus, Holliston, Mass.). Human T-cells are activated from whole peripheral blood provided by ALLCELLS (Alameda, Calif.) in X-Vivo-15 medium (Lonza, Basel, Switzerland) supplemented with 20 ng/ml human IL-2 (Miltenyi Biotech, San Diego, Calif.), 5% human AB serum (Gemini Bio-Products, West San Francisco, Calif.) and Dynabeads Human T-activator CD3/CD28 at a 1:1 bead:cell ratio (Thermo Fisher Scientific, Grand Island, N.Y.). Beads are removed after 3 days and 5×106 cells are electroporated with 10 μg mRNA of each of the two adequate TALEN® using Cytopulse (BTX Harvard Apparatus, Holliston, Mass.) by applying two 0.1 mS pulses at 3,000 V/cm followed by four 0.2 mS pulses at 325 V/cm in 0.4 cm gap cuvettes in a final volume of 200 μl of “cytoporation medium T” (BTX Harvard Apparatus, Holliston, Mass.). Cells are immediately diluted in X-Vivo-15 media with 20 ng/mL IL-2 and incubated at 37° C. with 5% CO2. After two hours, cells are incubated with AAV6 particles at 5×10\hat{ }5 viral genomes (vg) per cell (370C, 16 hours). Cells are passaged and maintained in X-Vivo-15 medium supplemented with 5% human AB serum and 20 ng/mL IL-2 until examined for expression by flow cytometry.

### Example 6. In Vivo Induction and Anti-Tumoral Effect of Engineered A/D-CAR Cells

Mice model of human hematopoietic or solid cancer were used (Huse, Jason T, and Eric C Holland. “Genetically Engineered Mouse Models of Brain Cancer and the Promise of Preclinical Testing.” Brain Pathology (Zurich, Switzerland) 19.1 (2009): 132-143. PMC. Web. 22 Nov. 2016 Cheng L, Ramesh A V, Flesken-Nikitin A, Choi J, Nikitin A Y. Mouse models for cancer stem cell research. Toxicologic pathology. 2010; 38(1):62-71. J Clin Invest. 2011 January; 121(1):384-95. doi: 10.1172/JC141495. Epub 2010 Dec. 13. Human acute myelogenous leukemia stem cells are rare and heterogeneous when assayed in NOD/SCID/IL2Ryc-deficient mice. Sarry J E1, Murphy K, Perry R, Sanchez P V, Secreto A, Keefer C, Swider C R, Strzelecki A C, Cavelier C, Récher C, Mansat-De Mas V, Delabesse E, Danet-Desnoyers G, Carroll M. doi:10.1177/0192623309354109.). These models were used to quantify the anti-tumoral activity of the A/D-CAR T cells of the invention using target cells expressing one (A), the other one (D) or both targets (A and D) recognized by the A-CAR and D-CAR.

Thus, mice were inoculated (right paw or intracerebrally) with cancer cells expressing CD22 or CD123 (A antigen) and/or CD34, EGFRVIII (D antigen) (A and/or D antigen) and optionally (left paw) with cancer cells expressing D antigens (CD34, EGFRVIII) alone. Mice received primary immune T cells engineered (TRAC KO) and expressing A-CARs (targeting A) or (D-CARs (targeting D) or A+D Cart T cells (targeting both A and D). In certain experiments, Anti-CD19, anti-CD33 CAR T cells were used as a positive control.

In the case of a solid tumor (CD22+glioma) or in a case of a “liquid” (hematopoietic CD123+AML) tumor, the A/D-CAR cells (expressing both types of CARs) of the invention significantly decreases tumor mass—as compared to control (unrelated CAR T) and at least similar to a CD22 or CD123-CAR expressing cell (no D-CAR), or induces 15% more decrease of tumor mass, as compared to a CAR-A/D expressing cell when A is not D, and when the ratio of CAR-A/CAR-D at the cell surface is 1:1, is >1, more preferably A-CAR/D-CAR at the cell surface is from 2 to 5. In the present setting, a decrease of 95% of the tumor mass was observed at day 28 when liquid tumor cells expressed both A and D or A alone. In mice with tumor expressing D alone, no significant tumor loss was observed. The results show that in mice engrafted with one tumor expressing A and one tumor expressing A+D, at two different locations, injection of A/D-CAR T-cells allowed to control only the A tumor. Injection of A-CAR T-cells allowed controlling both the A and A/D tumors. Injection of D-CAR T-cells did not allow controlling any of the two tumors.

### Example 7: AAV Driven Homologous Recombination in Human Primary T-Cells at Various Loci Under Control of Endogenous Promoters with Knock-Out of the Endogenous Gene

### Introduction

Sequence specific endonuclease reagents, such as TALEN® (Cellectis, 8 rue de la Croix Jarry, 75013 PARIS) enable the site-specific induction of double-stranded breaks (DSBs) in the genome at desired loci. Repair of DSBs by cellular enzymes occurs mainly through two pathways: non-homologous end joining (NHEJ) and homology directed repair (HDR). HDR uses a homologous piece of DNA (template DNA) to repair the DSB by recombination and can be used to introduce any genetic sequence comprised in the template DNA. As shown therein, said template DNA can be delivered by recombinant adeno-associated virus (rAAV) along with an engineered nuclease such as TALEN® to introduce a site-specific DSB.

### Design of the Integration Matrices

1.1. Insertion of an Apoptosis CAR in an Upregulated Locus with Knock-Out of the Endogenous PD1 Gene Coding Sequence

The location of the TALEN target site has been designed to be located in the targeted endogenous PDCD1 gene (Programmed cell death protein 1 referred to as PD1—Uniprot # Q15116). The sequence encompassing 1000 bp upstream and downstream the TALEN targets is given in SEQ ID NO: 112 and SEQ ID NO: 113. Target sequences of the TALEN (SEQ ID NO: 114 and SEQ ID NO: 115) is given in SEQ ID NO: 116. The integration matrix is designed to be composed of a sequence (300 bp) homologous to the endogenous gene upstream of the TALEN site (SEQ ID NO: 112), followed by a 2A regulatory element (SEQ ID NO: 117), followed by a sequence encoding an apoptosis inducing CAR without the start codon (SEQ ID NO: 118), followed by a STOP codon (TAG), followed by a polyadenylation sequence (SEQ ID NO: 119), followed by a sequence (1000 bp) homologous to the endogenous gene downstream of the TALEN site (SEQ ID NO: 113). The insertion matrix is subsequently cloned into a promoterless rAAV vector and used to produce AAV6.

1.2 Insertion of an Interleukin in an Upregulated Locus with Knock-Out of the Endogenous Gene

The location of the TALEN target site is designed to be located in the targeted endogenous PDCD1 gene (Programmed cell death protein 1, PD1). The sequence encompassing 1000 bp upstream and downstream the TALEN targets is given in SEQ ID NO: 112 and SEQ ID NO: 113. Target sequences of the TALEN (SEQ ID NO: 114 and SEQ ID NO:115) is given in SEQ ID NO: 116. The integration matrix is designed to be composed of a sequence (300 bp) homologous to the endogenous gene upstream of the TALEN site (SEQ ID NO: 112), followed by a 2A regulatory element (SEQ ID NO: 117), followed by a sequence encoding an engineered single-chained human IL-12 p35 (SEQ ID NO: 120) and p40 (SEQ ID NO: 121) subunit fusion protein, followed by a STOP codon (TAG), followed by a polyadenylation sequence (SEQ ID NO: 119), followed by a sequence (1000 bp) homologous to the endogenous gene downstream of the TALEN site (SEQ ID NO: 113). The insertion matrix is subsequently cloned into a promoterless rAAV vector and used to produce AAV6.

1.3 Insertion of an Apoptosis CAR in a Weakly Expressed Locus without Knocking Out the Endogenous Gene—N-Terminal Insertion

The location of the TALEN target site is designed to be located as close as possible to the start codon of the targeted endogenous LCK gene (LCK, LCK proto-oncogene, Src family tyrosine kinase [Homo sapiens (human)]). The sequence encompassing 1000 bp upstream and downstream the start codon is given in SEQ ID NO: 122 and SEQ ID NO: 123. The integration matrix is designed to be composed of a sequence (1000 bp) homologous to the endogenous gene upstream of the start codon, followed by a sequence encoding an apoptosis inducing CAR containing a start codon (SEQ ID NO: 124), followed by a 2A regulatory element (SEQ ID NO: 117), followed by a sequence (1000 bp) homologous to the endogenous gene downstream of the start codon (SEQ ID NO: 123). The insertion matrix is subsequently cloned into a promoterless rAAV vector and used to produce AAV6.

1.4 Insertion of an Apoptosis CAR in a Weakly Expressed Locus without Knocking Out the Endogenous Gene—C-Terminal Insertion

The location of the TALEN target site is designed to be located as close as possible to the stop codon of the targeted endogenous LCK gene (LCK, LCK proto-oncogene, Src family tyrosine kinase [Homo sapiens (human)]). The sequence encompassing 1000 bp upstream and downstream the stop codon is given in SEQ ID NO: 125 and SEQ ID NO: 126. The integration matrix is designed to be composed of a sequence (1000 bp) homologous to the endogenous gene upstream of the stop codon, followed by a 2A regulatory element (SEQ ID NO: 119), followed by a sequence encoding an apoptosis inducing CAR without the start codon (SEQ ID NO: 118), followed by a STOP codon (TAG), followed by a sequence (1000 bp) homologous to the endogenous gene downstream of the stop codon (SEQ ID NO.116). The insertion matrix is subsequently cloned into a promoterless rAAV vector and used to produce AAV6.

### Expression of the Sequence-Specific Nuclease Reagents in the Transduced Cells

TALEN® mRNA is synthesized using the mMessage mMachine T7 Ultra kit (Thermo Fisher Scientific, Grand Island, N.Y.) as each TALEN is cloned downstream of a T7 promoter, purified using RNeasy columns (Qiagen, Valencia, Calif.) and eluted in “cytoporation medium T” (Harvard Apparatus, Holliston, Mass.). Human T-cells are collected and activated from whole peripheral blood provided by ALLCELLS (Alameda, Calif.) in X-Vivo-15 medium (Lonza, Basel, Switzerland) supplemented with 20 ng/ml human IL-2 (Miltenyi Biotech, San Diego, Calif.), 5% human AB serum (Gemini Bio-Products, West San Francisco, Calif.) and Dynabeads Human T-activator CD3/CD28 at a 1:1 bead:cell ratio (Thermo Fisher Scientific, Grand Island, N.Y.). Beads are removed after 3 days and 5×10⁶ cells are electroporated with 10 μg mRNA of each of the two adequate TALEN® using Cytopulse (BTX Harvard Apparatus, Holliston, Mass.) by applying two 0.1 mS pulses at 3,000 V/cm followed by four 0.2 mS pulses at 325 V/cm in 0.4 cm gap cuvettes in a final volume of 200 μl of “cytoporation medium T” (BTX Harvard Apparatus, Holliston, Mass.). Cells are immediately diluted in X-Vivo-15 media with 20 ng/mL IL-2 and incubated at 37° C. with 5% CO₂. After two hours, cells are incubated with AAV6 particles at 3×10\hat{ }5 viral genomes (vg) per cell (37° C., 16 hours). Cells are passaged and maintained in X-Vivo-15 medium supplemented with 5% human AB serum and 20 ng/mL IL-2 until examined by flow cytometry for expression of the respective inserted gene sequences.

Sequences Referred to in Example 7

| | Ref. | |
|---|---|---|
| Sequence name | sequences | Polynucleotide or polypeptide sequences |
| PD1 left | SEQ ID NO: | CCAAGCCCTGACCCTGGCAGGCATATGTTTCAGGAGGTCCTTGTCT |
| homology | 112 | TGGGAGCCCAGGGTCGGGGGCCCCGTGTCTGTCCACATCCGAGTC |
| | | AATGGCCCATCTCGTCTCTGAAGCATCTTTGCTGTGAGCTCTAGTCC |
| | | CCACTGTCTTGCTGGAAAATGTGGAGGCCCCACTGCCCACTGCCCA |
| | | GGGCAGCAATGCCCATACCACGTGGTCCCAGCTCCGAGCTTGTCCT |
| | | GAAAAGGGGGCAAAGACTGGACCCTGAGCCTGCCAAGGGGCCAC |
| | | ACTCCTCCCAGGGCTGGGGTCTCCATGGGCAGCCCCCCACCCACCC |
| | | AGACCAGTTACACTCCCCTGTGCCAGAGCAGTGCAGACAGGACCA |
| | | GGCCAGGATGCCCAAGGGTCAGGGGCTGGGGATGGGTAGCCCCC |
| | | AAACAGCCCTTTCTGGGGGAACTGGCCTCAACGGGGAAGGGGGTG |
| | | AAGGCTCTTAGTAGGAAATCAGGGAGACCCAAGTCAGAGCCAGGT |
| | | GCTGTGCAGAAGCTGCAGCCTCACGTAGAAGGAAGAGGCTCTGCA |
| | | GTGGAGGCCAGTGCCCATCCCCGGGTGGCAGAGGCCCCAGCAGAG |
| | | ACTTCTCAATGACATTCCAGCTGGGGTGGCCCTTCCAGAGCCCTTG |
| | | CTGCCCGAGGGATGTGAGCAGGTGGCCGGGGAGGCTTTGTGGGG |
| | | CCACCCAGCCCCTTCCTCACCTCTCTCCATCTCTCAGACTCCCCAGAC |
| | | AGGCCCTGGAACCCCCCCACCTTCTCCCCAGCCCTGCTCGTGGTGAC |
| | | CGAAGGGGACAACGCCACCTTCACCTGCAGCTTCTCCAACACATCG |
| | | GAGAGCTTCGTGCTAAACTGGTACCGCATGAGCCCCAGCAACCAG |
| | | ACGGACAAGCTGGCCGCCTTCCCCGAGGACCGCAGCCAGCCCGGC |
| | | CAGGACTGCCGCTTCCGTGTCACACAACTGCCCAACGGGCGTGACT |
| | | TCCACATGAGCGTGGTCAGGGCCCGGCGCAATGACAGCGGCACC |
| PD1 right | SEQ ID NO: | GCCTGCGGGCAGAGCTCAGGGTGACAGGTGCGGCCTCGGAGGCC |
| homology | 113 | CCGGGGCAGGGGTGAGCTGAGCCGGTCCTGGGGTGGGTGTCCCCT |
| | | CCTGCACAGGATCAGGAGCTCCAGGGTCGTAGGGCAGGGACCCCC |
| | | CAGCTCCAGTCCAGGGCTCTGTCCTGCACCTGGGGAATGGTGACCG |
| | | GCATCTCTGTCCTCTAGCTCTGGAAGCACCCCAGCCCCTCTAGTCTG |
| | | CCCTCACCCCTGACCCTGACCCTCCACCCTGACCCCGTCCTAACCCCT |
| | | GACCTTTGTGCCCTTCCAGAGAGAAGGGCAGAAGTGCCCACAGCC |
| | | CACCCCAGCCCCTCACCCAGGCCAGCCGGCCAGTTCCAAACCCTGG |
| | | TGGTTGGTGTCGTGGGCGGCCTGCTGGGCAGCCTGGTGCTGCTAG |
| | | TCTGGGTCCTGGCCGTCATCTGCTCCCGGGCCGCACGAGGTAACGT |
| | | CATCCCAGCCCCTCGGCCTGCCCTGCCCTAACCCTGCTGGCGGCCCT |
| | | CACTCCCGCCTCCCCTTCCTCCACCCTTCCCTCACCCCACCCCACCTC |
| | | CCCCCATCTCCCCGCCAGGCTAAGTCCCTGATGAAGGCCCCTGGAC |
| | | TAAGACCCCCCACCTAGGAGCACGGCTCAGGGTCGGCCTGGTGAC |
| | | CCCAAGTGTGTTTCTCTGCAGGGACAATAGGAGCCAGGCGCACCG |
| | | GCCAGCCCCTGGTGAGTCTCACTCTTTTCCTGCATGATCCACTGTGC |
| | | CTTCCTTCCTGGGTGGGCAGAGGTGGAAGGACAGGCTGGGACCAC |
| | | ACGGCCTGCAGGACTCACATTCTATTATAGCCAGGACCCCACCTCCC |
| | | CAGCCCCCAGGCAGCAACCTCAATCCCTAAAGCCATGATCTGGGGC |
| | | CCCAGCCCACCTGCGGTCTCCGGGGGTGCCCGGCCCATGTGTGTGC |
| | | CTGCCTGCGGTCTCCAGGGGTGCCTGGCCCACGCGTGTGCCCGCCT |
| | | GCGGTCTCTGGGGGTGCCCGGCCCACATATGTGCC |
| PD1_T3C-L2 | SEQ ID NO: | ATGGGCGATCCTAAAAAGAAACGTAAGGTCATCGATATCGCCGATC |
| | 114 | TACGCACGCTCGGCTACAGCCAGCAGCAACAGGAGAAGATCAAAC |
| | | CGAAGGTTCGTTCGACAGTGGCGCAGCACCACGAGGCACTGGTCG |
| | | GCCACGGGTTTACACACGCGCACATCGTTGCGTTAAGCCAACACCC |
| | | GGCAGCGTTAGGGACCGTCGCTGTCAAGTATCAGGACATGATCGC |
| | | AGCGTTGCCAGAGGCGACACACGAAGCGATCGTTGGCGTCGGCAA |
| | | ACAGTGGTCCGGCGCACGCGCTCTGGAGGCCTTGCTCACGGTGGC |
| | | GGGAGAGTTGAGAGGTCCACCGTTACAGTTGGACACAGGCCAACT |
| | | TCTCAAGATTGCAAAACGTGGCGGCGTGACCGCAGTGGAGGCAGT |
| | | GCATGCATGGCGCAATGCACTGACGGGTGCCCCGCTCAACTTGACC |
| | | CCCGAGCAAGTGGTGGCTATCGCTTCCAAGCTGGGGGGAAAGCAG |
| | | GCCCTGGAGACCGTCCAGGCCCTTCTCCCAGTGCTTTGCCAGGCTC |
| | | ACGGACTGACCCCTGAACAGGTGGTGGCAATTGCCTCACACGACG |
| | | GGGGCAAGCAGGCACTGGAGACTGTCCAGCGGCTGCTGCCTGTCC |
| | | TCTGCCAGGCCCACGGACTCACTCCTGAGCAGGTCGTGGCCATTGC |
| | | CAGCCACGATGGGGGCAAACAGGCTCTGGAGACCGTGCAGCGCCT |
| | | CCTCCCAGTGCTGTGCCAGGCTCATGGGCTGACCCCACAGCAGGTC |
| | | GTCGCCATTGCCAGTAACGGCGGGGGGAAGCAGGCCCTCGAAACA |
| | | GTGCAGAGGCTGCTGCCCGTCTTGTGCCAAGCACACGGCCTGACAC |
| | | CCGAGCAGGTGGTGGCCATCGCCTCTCATGACGGCGGCAAGCAGG |
| | | CCCTTGAGACAGTGCAGAGACTGTTGCCCGTGTTGTGTCAGGCCCA |
| | | CGGGTTGACACCCCAGCAGGTGGTCGCCATCGCCAGCAATGGCGG |
| | | GGGAAAGCAGGCCCTTGAGACCGTGCAGCGGTTGCTTCCAGTGTT |
| | | GTGCCAGGCACACGGACTGACCCCTCAACAGGTGGTCGCAATCGC |
| | | CAGCTACAAGGGCGGAAAGCAGGCTCTGGAGACAGTGCAGCGCCT |
| | | CCTGCCCGTGCTGTGTCAGGCTCACGGACTGACACCACAGCAGGTG |
| | | GTCGCCATCGCCAGTAACGGGGGCGGCAAGCAGGCTTTGGAGACC |
| | | GTCCAGAGACTCCTCCCCGTCCTTTGCCAGGCCCACGGGTTGACAC |
| | | CTCAGCAGGTCGTCGCCATTGCCTCCAACAACGGGGGCAAGCAGG |
| | | CCCTCGAAACTGTGCAGAGGCTGCTGCCTGTGCTGTGCCAGGCTCA |
| | | TGGGCTGACACCCCAGCAGGTGGTGGCCATTGCCTCTAACAACGGC |
| | | GGCAAACAGGCACTGGAGACCGTGCAAAGGCTGCTGCCCGTCCTC |
| | | TGCCAAGCCCACGGGCTCACTCCACAGCAGGTCGTGGCCATCGCCT |
| | | CAAACAATGGCGGGAAGCAGGCCCTGGAGACTGTGCAAAGGCTGC |
| | | TCCCTGTGCTCTGCCAGGCACACGGACTGACCCCTCAGCAGGTGGT |
| | | GGCAATCGCTTCCAACAACGGGGGAAAGCAGGCCCTCGAAACCGT |
| | | GCAGCGCCTCCTCCCAGTGCTGTGCCAGGCACATGGCCTCACACCC |
| | | GAGCAAGTGGTGGCTATCGCCAGCCACGACGGAGGGAAGCAGGC |
| | | TCTGGAGACCGTGCAGAGGCTGCTGCCTGTCCTGTGCCAGGCCCAC |
| | | GGGCTTACTCCAGAGCAGGTCGTCGCCATCGCCAGTCATGATGGG |
| | | GGGAAGCAGGCCCTTGAGACAGTCCAGCGGCTGCTGCCAGTCCTTT |
| | | GCCAGGCTCACGGCTTGACTCCCGAGCAGGTCGTGGCCATTGCCTC |
| | | AAACATTGGGGGCAAACAGGCCCTGGAGACAGTGCAGGCCCTGCT |
| | | GCCCGTGTTGTGTCAGGCCCACGGCTTGACACCCCAGCAGGTGGTC |
| | | GCCATTGCCTCTAATGGCGGCGGGAGACCCGCCTTGGAGAGCATT |
| | | GTTGCCCAGTTATCTCGCCCTGATCCGGCGTTGGCCGCGTTGACCA |
| | | ACGACCACCTCGTCGCCTTGGCCTGCCTCGGCGGGCGTCCTGCGCT |
| | | GGATGCAGTGAAAAAGGGATTGGGGGATCCTATCAGCCGTTCCCA |
| | | GCTGGTGAAGTCCGAGCTGGAGGAGAAGAAATCCGAGTTGAGGC |
| | | ACAAGCTGAAGTACGTGCCCCACGAGTACATCGAGCTGATCGAGA |
| | | TCGCCCGGAACAGCACCCAGGACCGTATCCTGGAGATGAAGGTGA |
| | | TGGAGTTCTTCATGAAGGTGTACGGCTACAGGGGCAAGCACCTGG |
| | | GCGGCTCCAGGAAGCCCGACGGCGCCATCTACACCGTGGGCTCCC |
| | | CCATCGACTACGGCGTGATCGTGGACACCAAGGCCTACTCCGGCG |
| | | GCTACAACCTGCCCATCGGCCAGGCCGACGAAATGCAGAGGTACG |
| | | TGGAGGAGAACCAGACCAGGAACAAGCACATCAACCCCAACGAGT |
| | | GGTGGAAGGTGTACCCCTCCAGCGTGACCGAGTTCAAGTTCCTGTT |
| | | CGTGTCCGGCCACTTCAAGGGCAACTACAAGGCCCAGCTGACCAG |
| | | GCTGAACCACATCACCAACTGCAACGGCGCCGTGCTGTCCGTGGAG |
| | | GAGCTCCTGATCGGCGGCGAGATGATCAAGGCCGGCACCCTGACC |
| | | CTGGAGGAGGTGAGGAGGAAGTTCAACAACGGCGAGATCAACTTC |
| | | GCGGCCGACTGATAA |
| PD1T3R | SEQ ID NO: | ATGGGCGATCCTAAAAAGAAACGTAAGGTCATCGATATCGCCGATC |
| | 115 | TACGCACGCTCGGCTACAGCCAGCAGCAACAGGAGAAGATCAAAC |
| | | CGAAGGTTCGTTCGACAGTGGCGCAGCACCACGAGGCACTGGTCG |
| | | GCCACGGGTTTACACACGCGCACATCGTTGCGTTAAGCCAACACCC |
| | | GGCAGCGTTAGGGACCGTCGCTGTCAAGTATCAGGACATGATCGC |
| | | AGCGTTGCCAGAGGCGACACACGAAGCGATCGTTGGCGTCGGCAA |
| | | ACAGTGGTCCGGCGCACGCGCTCTGGAGGCCTTGCTCACGGTGGC |
| | | GGGAGAGTTGAGAGGTCCACCGTTACAGTTGGACACAGGCCAACT |
| | | TCTCAAGATTGCAAAACGTGGCGGCGTGACCGCAGTGGAGGCAGT |
| | | GCATGCATGGCGCAATGCACTGACGGGTGCCCCGCTCAACTTGACC |
| | | CCCGAGCAAGTCGTCGCAATCGCCAGCCATGATGGAGGGAAGCAA |
| | | GCCCTCGAAACCGTGCAGCGGTTGCTTCCTGTGCTCTGCCAGGCCC |
| | | ACGGCCTTACCCCTCAGCAGGTGGTGGCCATCGCAAGTAACGGAG |
| | | GAGGAAAGCAAGCCTTGGAGACAGTGCAGCGCCTGTTGCCCGTGC |
| | | TGTGCCAGGCACACGGCCTCACACCAGAGCAGGTCGTGGCCATTG |
| | | CCTCCCATGACGGGGGGAAACAGGCTCTGGAGACCGTCCAGAGGC |
| | | TGCTGCCCGTCCTCTGTCAAGCTCACGGCCTGACTCCCCAACAAGTG |
| | | GTCGCCATCGCCTCTAATGGCGGCGGGAAGCAGGCACTGGAAACA |
| | | GTGCAGAGACTGCTCCCTGTGCTTTGCCAAGCTCATGGGTTGACCC |
| | | CCCAACAGGTCGTCGCTATTGCCTCAAACGGGGGGGGCAAGCAGG |
| | | CCCTTGAGACTGTGCAGAGGCTGTTGCCAGTGCTGTGTCAGGCTCA |
| | | CGGGCTCACTCCACAACAGGTGGTCGCAATTGCCAGCAACGGCGG |
| | | CGGAAAGCAAGCTCTTGAAACCGTGCAACGCCTCCTGCCCGTGCTC |
| | | TGTCAGGCTCATGGCCTGACACCACAACAAGTCGTGGCCATCGCCA |
| | | GTAATAATGGCGGGAAACAGGCTCTTGAGACCGTCCAGAGGCTGC |
| | | TCCCAGTGCTCTGCCAGGCACACGGGCTGACCCCCGAGCAGGTGG |
| | | TGGCTATCGCCAGCAATATTGGGGGCAAGCAGGCCCTGGAAACAG |
| | | TCCAGGCCCTGCTGCCAGTGCTTTGCCAGGCTCACGGGCTCACTCC |
| | | CCAGCAGGTCGTGGCAATCGCCTCCAACGGCGGAGGGAAGCAGGC |
| | | TCTGGAGACCGTGCAGAGACTGCTGCCCGTCTTGTGCCAGGCCCAC |
| | | GGACTCACACCTGAACAGGTCGTCGCCATTGCCTCTCACGATGGGG |
| | | GCAAACAAGCCCTGGAGACAGTGCAGCGGCTGTTGCCTGTGTTGT |
| | | GCCAAGCCCACGGCTTGACTCCTCAACAAGTGGTCGCCATCGCCTC |
| | | AAATGGCGGCGGAAAACAAGCTCTGGAGACAGTGCAGAGGTTGCT |
| | | GCCCGTCCTCTGCCAAGCCCACGGCCTGACTCCCCAACAGGTCGTC |
| | | GCCATTGCCAGCAACAACGGAGGAAAGCAGGCTCTCGAAACTGTG |
| | | CAGCGGCTGCTTCCTGTGCTGTGTCAGGCTCATGGGCTGACCCCCG |
| | | AGCAAGTGGTGGCTATTGCCTCTAATGGAGGCAAGCAAGCCCTTG |
| | | AGACAGTCCAGAGGCTGTTGCCAGTGCTGTGCCAGGCCCACGGGC |
| | | TCACACCCCAGCAGGTGGTCGCCATCGCCAGTAACAACGGGGGCA |
| | | AACAGGCATTGGAAACCGTCCAGCGCCTGCTTCCAGTGCTCTGCCA |
| | | GGCACACGGACTGACACCCGAACAGGTGGTGGCCATTGCATCCCA |
| | | TGATGGGGGCAAGCAGGCCCTGGAGACCGTGCAGAGACTCCTGCC |
| | | AGTGTTGTGCCAAGCTCACGGCCTCACCCCTCAGCAAGTCGTGGCC |
| | | ATCGCCTCAAACGGGGGGGGCCGGCCTGCACTGGAGAGCATTGTT |
| | | GCCCAGTTATCTCGCCCTGATCCGGCGTTGGCCGCGTTGACCAACG |
| | | ACCACCTCGTCGCCTTGGCCTGCCTCGGCGGGCGTCCTGCGCTGGA |
| | | TGCAGTGAAAAAGGGATTGGGGGATCCTATCAGCCGTTCCCAGCT |
| | | GGTGAAGTCCGAGCTGGAGGAGAAGAAATCCGAGTTGAGGCACA |
| | | AGCTGAAGTACGTGCCCCACGAGTACATCGAGCTGATCGAGATCG |
| | | CCCGGAACAGCACCCAGGACCGTATCCTGGAGATGAAGGTGATGG |
| | | AGTTCTTCATGAAGGTGTACGGCTACAGGGGCAAGCACCTGGGCG |
| | | GCTCCAGGAAGCCCGACGGCGCCATCTACACCGTGGGCTCCCCCAT |
| | | CGACTACGGCGTGATCGTGGACACCAAGGCCTACTCCGGCGGCTA |
| | | CAACCTGCCCATCGGCCAGGCCGACGAAATGCAGAGGTACGTGGA |
| | | GGAGAACCAGACCAGGAACAAGCACATCAACCCCAACGAGTGGTG |
| | | GAAGGTGTACCCCTCCAGCGTGACCGAGTTCAAGTTCCTGTTCGTG |
| | | TCCGGCCACTTCAAGGGCAACTACAAGGCCCAGCTGACCAGGCTG |
| | | AACCACATCACCAACTGCAACGGCGCCGTGCTGTCCGTGGAGGAG |
| | | CTCCTGATCGGCGGCGAGATGATCAAGGCCGGCACCCTGACCCTG |
| | | GAGGAGGTGAGGAGGAAGTTCAACAACGGCGAGATCAACTTCGC |
| | | GGCCGACTGATAA |
| PD1-T3 | SEQ ID NO: | TACCTCTGTGGGGCCATCTCCCTGGCCCCCAAGGCGCAGATCAAAG |
| | 116 | AGA |
| 2A-element | SEQ ID NO: | TCCGGTGAGGGCAGAGGAAGTCTTCTAACATGCGGTGACGTGGAG |
| | 117 | GAGAATCCGGGCCCC |
| apoptosis CAR | SEQ ID NO: | GCTTTGCCTGTCACTGCCTTGCTGCTTCCACTTGCTCTGTTGTTGCAC |
| (without start | 118 | GCCGCAAGACCCGAGGTCAAGCTCCAGGAAAGCGGACCAGGGCT |
| codon) | | GGTGGCCCCTAGTCAGTCATTGAGCGTCACTTGCACCGTCAGCGGC |
| | | GTGTCTCTGCCCGATTACGGCGTGAGCTGGATCAGACAGCCCCCAA |
| | | GGAAGGGACTGGAGTGGCTGGGCGTCATCTGGGGGAGCGAGACT |
| | | ACCTACTACAACAGCGCCCTGAAGAGCAGGCTGACCATCATTAAGG |
| | | ACAACTCCAAGTCCCAGGTCTTTCTGAAAATGAACAGCCTGCAGAC |
| | | TGATGACACTGCCATCTACTACTGCGCCAAGCATTACTACTACGGG |
| | | GGCAGCTACGCTATGGACTACTGGGGGCAGGGGACCTCTGTCACA |
| | | GTGTCAAGTGGCGGAGGAGGCAGTGGCGGAGGGGGAAGTGGGG |
| | | GCGGCGGCAGCGACATCCAGATGACCCAGACAACATCCAGCCTCTC |
| | | CGCCTCTCTGGGCGACAGAGTGACAATCAGCTGCCGGGCCAGTCA |
| | | GGACATCAGCAAGTATCTCAATTGGTACCAGCAGAAACCAGACGG |
| | | GACAGTGAAATTGCTGATCTACCACACATCCAGGCTGCACTCAGGA |
| | | GTCCCCAGCAGGTTTTCCGGCTCCGGCTCCGGGACAGATTACAGTC |
| | | TGACCATTTCCAACCTGGAGCAGGAGGATATTGCCACATACTTTTG |
| | | CCAGCAAGGCAACACTCTGCCCTATACCTTCGGCGGAGGCACAAAA |
| | | CTGGAGATTACTCGGTCGGATCCCGAGCCCAAATCTCCTGACAAAA |
| | | CTCACACATGCCCACCGTGCCCAGCACCTCCCGTGGCCGGCCCGTC |
| | | AGTGTTCCTCTTCCCCCCAAAACCCAAGGACACCCTCATGATCGCCC |
| | | GGACCCCTGAGGTCACATGCGTGGTGGTGGACGTGAGCCACGAGG |
| | | ACCCTGAGGTCAAGTTCAACTGGTACGTGGACGGCGTGGAGGTGC |
| | | ATAATGCCAAGACAAAGCCGCGGGAGGAGCAGTACAACAGCACGT |
| | | ACCGTGTGGTCAGCGTCCTCACCGTCCTGCACCAGGACTGGCTGAA |
| | | TGGCAAGGAGTACAAGTGCAAGGTGTCCAACAAAGCCCTCCCAGC |
| | | CCCCATCGAGAAAACCATCTCCAAAGCCAAAGGGCAGCCCCGAGA |
| | | ACCACAGGTGTACACCCTGCCCCCATCCCGGGATGAGCTGACCAAG |
| | | AACCAGGTCAGCCTGACCTGCCTGGTCAAAGGCTTCTATCCCAGCG |
| | | ACATCGCCGTGGAGTGGGAGAGCAATGGGCAACCGGAGAACAAC |
| | | TACAAGACCACGCCTCCCGTGCTGGACTCCGACGGCTCCTTCTTCCT |
| | | CTACAGCAAGCTCACCGTGGACAAGAGCAGGTGGCAGCAGGGGA |
| | | ACGTGTTCTCATGCTCCGTGATGCATGAGGCCCTGCACAATCACTAT |
| | | ACCCAGAAATCTCTGAGTCTGAGCCCAGGCAAGAAGGATATTTTGG |
| | | GGTGGCTTTGCCTTCTTCTTTTGCCAATTCCACTAATTGTTTGGGTG |
| | | AAGAGAAAGGAAGTACAGAAAACATGCAGAAAGCACAGAAAGGA |
| | | AAACCAAGGTTCTCATGAATCTCCAACCTTAAATCCTGAAACAGTG |
| | | GCAATAAATTTATCTGATGTTGACTTGAGTAAATATATCACCACTAT |
| | | TGCTGGAGTCATGACACTAAGTCAAGTTAAAGGCTTTGTTCGAAAG |
| | | AATGGTGTCAATGAAGCCAAAATAGATGAGATCAAGAATGACAAT |
| | | GTCCAAGACACAGCAGAACAGAAAGTTCAACTGCTTCGTAATTGGC |
| | | ATCAACTTCATGGAAAGAAAGAAGCGTATGACACATTGATTGCAGA |
| | | TCTCAAAAAAGCCAATCTTTGTACTCTTGCAGAGAAAATTCAGACTA |
| | | TCATCCTCAAGGACATTACTAGTGACTCAGAAAATTCAAACTTCAGA |
| | | AATGAAATCCAGAGCTTGGTCGAA |
| BGH polyA | SEQ ID NO: | TCTAGAGGGCCCGTTTAAACCCGCTGATCAGCCTCGACTGTGCCTT |
| | 119 | CTAGTTGCCAGCCATCTGTTGTTTGCCCCTCCCCCGTGCCTTCCTTGA |
| | | CCCTGGAAGGTGCCACTCCCACTGTCCTTTCCTAATAAAATGAGGA |
| | | AATTGCATCGCATTGTCTGAGTAGGTGTCATTCTATTCTGGGGGGT |
| | | GGGGTGGGGCAGGACAGCAAGGGGGAGGATTGGGAAGACAATA |
| | | GCAGGCATGCTGGGGATGCGGTGGGCTCTATGACTAGTGGCGAAT |
| | | TC |
| Interleukin-12 | SEQ ID NO: | MCPARSLLLVATLVLLDHLSLARNLPVATPDPGMFPCLHHSQNLLRAV |
| subunit alpha | 120 | SNMLQKARQTLEFYPCTSEEIDHEDITKDKTSTVEACLPLELTKNESCLN |
| | | SRETSFITNGSCLASRKTSFMMALCLSSIYEDLKMYQVEFKTMNAKLL |
| | | MDPKRQIFLDQNMLAVIDELMQALNFNSETVPQKSSLEEPDFYKTKIK |
| | | LCILLHAFRIRAVTIDRVMSYLNAS |
| Interleukin-12 | SEQ ID NO: | MCHQQLVISWFSLVFLASPLVAIWELKKDVYVVELDWYPDAPGEMV |
| subunit beta | 121 | VLTCDTPEEDGITWTLDQSSEVLGSGKTLTIQVKEFGDAGQYTCHKGG |
| | | EVLSHSLLLLHKKEDGIWSTDILKDQKEPKNKTFLRCEAKNYSGRFTCW |
| | | WLTTISTDLTFSVKSSRGSSDPQGVTCGAATLSAERVRGDNKEYEYSVE |
| | | CQEDSACPAAEESLPIEVMVDAVHKLKYENYTSSFFIRDIIKPDPPKNLQ |
| | | LKPLKNSRQVEVSWEYPDTWSTPHSYFSLTFCVQVQGKSKREKKDRVF |
| | | TDKTSATVICRKNASISVRAQDRYYSSSWSEWASVPCS |
| Lck left | SEQ ID NO: | GGGATAGGGGGTGCCTCTGTGTGTGTGTGTGAGAGTGTGTGTGTG |
| homology | 122 | TAGGGTGTGTATATGTATAGGGTGTGTGTGAGTGTGTGTGTGTGA |
| | | GAGAGTGTGTGTGTGGCAGAATAGACTGCGGAGGTGGATTTCATC |
| | | TTGATATGAAAGGTCTGGAATGCATGGTACATTAAACTTTGAGGAC |
| | | AGCGCTTTCCAAGCACTCTGAGGAGCAGCCCTAGAGAAGGAGGAG |
| | | CTGCAGGGACTCCGGGGGCTTCAAAGTGAGGGCCCCACTCTGCTTC |
| | | AGGCAAAACAGGCACACATTTATCACTTTATCTATGGAGTTCTGCTT |
| | | GATTTCATCAGACAAAAAATTTCCACTGCTAAAACAGGCAAATAAA |
| | | CAAAAAAAAAGTTATGGCCAACAGAGTCACTGGAGGGTTTTCTGCT |
| | | GGGGAGAAGCAAGCCCGTGTTTGAAGGAACCCTGTGAGATGACTG |
| | | TGGGCTGTGTGAGGGGAACAGCGGGGGCTTGATGGTGGACTTCG |
| | | GGAGCAGAAGCCTCTTTCTCAGCCTCCTCAGCTAGACAGGGGAATT |
| | | ATAATAGGAGGTGTGGCGTGCACACCTCTCCAGTAGGGGAGGGTC |
| | | TGATAAGTCAGGTCTCTCCCAGGCTTGGGAAAGTGTGTGTCATCTC |
| | | TAGGAGGTGGTCCTCCCAACACAGGGTACTGGCAGAGGGAGAGG |
| | | GAGGGGGCAGAGGCAGGAAGTGGGTAACTAGACTAACAAAGGTG |
| | | CCTGTGGCGGTTTGCCCATCCCAGGTGGGAGGGTGGGGCTAGGGC |
| | | TCAGGGGCCGTGTGTGAATTTACTTGTAGCCTGAGGGCTCAGAGG |
| | | GAGCACCGGTTTGGAGCTGGGACCCCCTATTTTAGCTTTTCTGTGG |
| | | CTGGTGAATGGGGATCCCAGGATCTCACAATCTCAGGTACTTTTGG |
| | | AACTTTCCAGGGCAAGGCCCCATTATATCTGATGTTGGGGGAGCAG |
| | | ATCTTGGGGGAGCCCCTTCAGCCCCCTCTTCCATTCCCTCAGGGACC |
| Ick right | SEQ ID NO: | GGCTGTGGCTGCAGCTCACACCCGGAAGATGACTGGATGGAAAAC |
| homology | 123 | ATCGATGTGTGTGAGAACTGCCATTATCCCATAGTCCCACTGGATG |
| | | GCAAGGGCACGGTAAGAGGCGAGACAGGGGCCTTGGTGAGGGAG |
| | | TTGGGTAGAGAATGCAACCCAGGAGAAAGAAATGACCAGCACTAC |
| | | AGGCCCTTGAAAGAATAGAGTGGCCCTCTCCCCTGAAATACAGAAA |
| | | GGAAAAGAGGCCCAGAGAGGGGAAGGGAATCTCCTAAGATCACA |
| | | CAGAAAGTAGTTGGTAAACTCAGGGATAACATCTAACCAGGCTGG |
| | | AGAGGCTGAGAGCAGAGCAGGGGGGAAGGGGGCCAGGGTCTGA |
| | | CCCAATCTTCTGCTTTCTGACCCCACCCTCATCCCCCACTCCACAGCT |
| | | GCTCATCCGAAATGGCTCTGAGGTGCGGGACCCACTGGTTACCTAC |
| | | GAAGGCTCCAATCCGCCGGCTTCCCCACTGCAAGGTGACCCCAGGC |
| | | AGCAGGGCCTGAAAGACAAGGCCTGCGGATCCCTGGCTGTTGGCT |
| | | TCCACCTCTCCCCCACCTACTTTCTCCCCGGTCTTGCCTTCCTTGTCCC |
| | | CCACCCTGTAACTCCAGGCTTCCTGCCGATCCCAGCTCGGTTCTCCC |
| | | TGATGCCCCTTGTCTTTACAGACAACCTGGTTATCGCTCTGCACAGC |
| | | TATGAGCCCTCTCACGACGGAGATCTGGGCTTTGAGAAGGGGGAA |
| | | CAGCTCCGCATCCTGGAGCAGTGAGTCCCTCTCCACCTTGCTCTGGC |
| | | GGAGTCCGTGAGGGAGCGGCGATCTCCGCGACCCGCAGCCCTCCT |
| | | GCGGCCCTTGACCAGCTCGGGGTGGCCGCCCTTGGGACAAAATTC |
| | | GAGGCTCAGTATTGCTGAGCCAGGGTTGGGGGAGGCTGGCTTAAG |
| | | GGGTGGAGGGGTCTTTGAGGGAGGGTCTCAGGTCGACGGCTGAG |
| | | CGAGCCACACTGACCCACCTCCGTGGCGCAGGAGCGGCGAGTG |
| apoptosis CAR | SEQ ID NO: | ATGGCTTTGCCTGTCACTGCCTTGCTGCTTCCACTTGCTCTGTTGTTG |
| (with start | 124 | CACGCCGCAAGACCCGAGGTCAAGCTCCAGGAAAGCGGACCAGG |
| codon) | | GCTGGTGGCCCCTAGTCAGTCATTGAGCGTCACTTGCACCGTCAGC |
| | | GGCGTGTCTCTGCCCGATTACGGCGTGAGCTGGATCAGACAGCCCC |
| | | CAAGGAAGGGACTGGAGTGGCTGGGCGTCATCTGGGGGAGCGAG |
| | | ACTACCTACTACAACAGCGCCCTGAAGAGCAGGCTGACCATCATTA |
| | | AGGACAACTCCAAGTCCCAGGTCTTTCTGAAAATGAACAGCCTGCA |
| | | GACTGATGACACTGCCATCTACTACTGCGCCAAGCATTACTACTACG |
| | | GGGGCAGCTACGCTATGGACTACTGGGGGCAGGGGACCTCTGTCA |
| | | CAGTGTCAAGTGGCGGAGGAGGCAGTGGCGGAGGGGGAAGTGG |
| | | GGGCGGCGGCAGCGACATCCAGATGACCCAGACAACATCCAGCCT |
| | | CTCCGCCTCTCTGGGCGACAGAGTGACAATCAGCTGCCGGGCCAGT |
| | | CAGGACATCAGCAAGTATCTCAATTGGTACCAGCAGAAACCAGAC |
| | | GGGACAGTGAAATTGCTGATCTACCACACATCCAGGCTGCACTCAG |
| | | GAGTCCCCAGCAGGTTTTCCGGCTCCGGCTCCGGGACAGATTACAG |
| | | TCTGACCATTTCCAACCTGGAGCAGGAGGATATTGCCACATACTTTT |
| | | GCCAGCAAGGCAACACTCTGCCCTATACCTTCGGCGGAGGCACAAA |
| | | ACTGGAGATTACTCGGTCGGATCCCGAGCCCAAATCTCCTGACAAA |
| | | ACTCACACATGCCCACCGTGCCCAGCACCTCCCGTGGCCGGCCCGT |
| | | CAGTGTTCCTCTTCCCCCCAAAACCCAAGGACACCCTCATGATCGCC |
| | | CGGACCCCTGAGGTCACATGCGTGGTGGTGGACGTGAGCCACGAG |
| | | GACCCTGAGGTCAAGTTCAACTGGTACGTGGACGGCGTGGAGGTG |
| | | CATAATGCCAAGACAAAGCCGCGGGAGGAGCAGTACAACAGCACG |
| | | TACCGTGTGGTCAGCGTCCTCACCGTCCTGCACCAGGACTGGCTGA |
| | | ATGGCAAGGAGTACAAGTGCAAGGTGTCCAACAAAGCCCTCCCAG |
| | | CCCCCATCGAGAAAACCATCTCCAAAGCCAAAGGGCAGCCCCGAG |
| | | AACCACAGGTGTACACCCTGCCCCCATCCCGGGATGAGCTGACCAA |
| | | GAACCAGGTCAGCCTGACCTGCCTGGTCAAAGGCTTCTATCCCAGC |
| | | GACATCGCCGTGGAGTGGGAGAGCAATGGGCAACCGGAGAACAA |
| | | CTACAAGACCACGCCTCCCGTGCTGGACTCCGACGGCTCCTTCTTCC |
| | | TCTACAGCAAGCTCACCGTGGACAAGAGCAGGTGGCAGCAGGGGA |
| | | ACGTGTTCTCATGCTCCGTGATGCATGAGGCCCTGCACAATCACTAT |
| | | ACCCAGAAATCTCTGAGTCTGAGCCCAGGCAAGAAGGATATTTTGG |
| | | GGTGGCTTTGCCTTCTTCTTTTGCCAATTCCACTAATTGTTTGGGTG |
| | | AAGAGAAAGGAAGTACAGAAAACATGCAGAAAGCACAGAAAGGA |
| | | AAACCAAGGTTCTCATGAATCTCCAACCTTAAATCCTGAAACAGTG |
| | | GCAATAAATTTATCTGATGTTGACTTGAGTAAATATATCACCACTAT |
| | | TGCTGGAGTCATGACACTAAGTCAAGTTAAAGGCTTTGTTCGAAAG |
| | | AATGGTGTCAATGAAGCCAAAATAGATGAGATCAAGAATGACAAT |
| | | GTCCAAGACACAGCAGAACAGAAAGTTCAACTGCTTCGTAATTGGC |
| | | ATCAACTTCATGGAAAGAAAGAAGCGTATGACACATTGATTGCAGA |
| | | TCTCAAAAAAGCCAATCTTTGTACTCTTGCAGAGAAAATTCAGACTA |
| | | TCATCCTCAAGGACATTACTAGTGACTCAGAAAATTCAAACTTCAGA |
| | | AATGAAATCCAGAGCTTGGTCGAA |
| Lck left | SEQ ID NO: | CTCATAACAATTCTATGAGGTAGGAACAGTTATTTACTCTATTTTCC |
| homology | 125 | AAATAAGGAAACTGGGCTCGCCCAAGGTTCCACAACTAACATGTGT |
| | | GTATTATTGAGCATTTAATTTACACCAGGGAAGCAGGTTGTGGTGG |
| | | TGTGCACCTGTTGTCCAGCTATTTAGGAGGCTGAGGTGAAAGGATC |
| | | ACTTGAACGGAGGAGTTCAAATTTGCAATGTGCTATGATTGTGCCT |
| | | GTGAACAGCTGCTGCACTCCAGCCTGGGCAACATAGTGAGATCCCT |
| | | TATCTAAAACATTTTTTTTAAGTAAATAATCAGGTGGGCACGGTGG |
| | | CTCACGCCTGTAATCCAGCACTTTGGGAGGCTGAGGCGGGCGGAT |
| | | CACCTGAGGTCAGGAGTTCAAGACCAGCCTGACCAACATGGAGAA |
| | | ACCCGTCTCTACTAAAAATACAAAATTAGCTTGGCGTGGTGGTGCA |
| | | TGCCTGTAATCCCAGCTACTCGAGAAGCTGAGGCAGGAGAATTGTT |
| | | TGAACCTGGGAGGTGGAGGTTGCGGTGAGCCGAGATCGCACCATT |
| | | GCACTCCAGCCTGGGCAACAAGAGTGAAATTGCATCTCAAAAAAA |
| | | AAGAAAAGGAAATAATCTATACCAGGCACTCCAAGTGGTGTGACT |
| | | GATATTCAACAAGTACCTCTAGTGTGACCTTACCATTGATGAAGACC |
| | | AAGATTCTTTTGGATTGGTGCTCACACTGTGCCAGTTAAATATTCCG |
| | | AACATTACCCTTGCCTGTGGGCTTCCAGTGCCTGACCTTGATGTCCT |
| | | TTCACCCATCAACCCGTAGGGATGACCAACCCGGAGGTGATTCAGA |
| | | ACCTGGAGCGAGGCTACCGCATGGTGCGCCCTGACAACTGTCCAG |
| | | AGGAGCTGTACCAACTCATGAGGCTGTGCTGGAAGGAGCGCCCAG |
| | | AGGACCGGCCCACCTTTGACTACCTGCGCAGTGTGCTGGAGGACTT |
| | | CTTCACGGCCACAGAGGGCCAGTACCAGCCTCAGCCT |
| Ick right | SEQ ID NO: | GAGGCCTTGAGAGGCCCTGGGGTTCTCCCCCTTTCTCTCCAGCCTG |
| homology | 126 | ACTTGGGGAGATGGAGTTCTTGTGCCATAGTCACATGGCCTATGCA |
| | | CATATGGACTCTGCACATGAATCCCACCCACATGTGACACATATGC |
| | | ACCTTGTGTCTGTACACGTGTCCTGTAGTTGCGTGGACTCTGCACAT |
| | | GTCTTGTACATGTGTAGCCTGTGCATGTATGTCTTGGACACTGTACA |
| | | AGGTACCCCTTTCTGGCTCTCCCATTTCCTGAGACCACAGAGAGAG |
| | | GGGAGAAGCCTGGGATTGACAGAAGCTTCTGCCCACCTACTTTTCT |
| | | TTCCTCAGATCATCCAGAAGTTCCTCAAGGGCCAGGACTTTATCTAA |
| | | TACCTCTGTGTGCTCCTCCTTGGTGCCTGGCCTGGCACACATCAGGA |
| | | GTTCAATAAATGTCTGTTGATGACTGTTGTACATCTCTTTGCTGTCC |
| | | ACTCTTTGTGGGTGGGCAGTGGGGGTTAAGAAAATGGTAATTAGG |
| | | TCACCCTGAGTTGGGGTGAAAGATGGGATGAGTGGATGTCTGGAG |
| | | GCTCTGCAGACCCCTTCAAATGGGACAGTGCTCCTCACCCCTCCCCA |
| | | AAGGATTCAGGGTGACTCCTACCTGGAATCCCTTAGGGAATGGGT |
| | | GCGTCAAAGGACCTTCCTCCCCATTATAAAAGGGCAACAGCATTTTT |
| | | TACTGATTCAAGGGCTATATTTGACCTCAGATTTTGTTTTTTTAAGG |
| | | CTAGTCAAATGAAGCGGCGGGAATGGAGGAGGAACAAATAAATCT |
| | | GTAACTATCCTCAGATTTTTTTTTTTTTTTGAGACTGGGTCTCACTTT |
| | | TTCATCCAGGCTGGAGTGCAGTCGCATGATCACGGCTCACTGTAGC |
| | | CTCAACCTCTCCAGCTCAAATGCTCCTCCTGTCTCAGCCTCCCGAGT |
| | | ACCTGGGACTACTTTCTTGAGGCCAGGAATTCAAGAACAGAGTAAG |
| | | ATCCTGGTCTCCAAAAAAAGTTTTAAA |

1.-67. (canceled)

68. A cell death inducing chimeric antigen receptor (CAR) comprising:
a) at least one ectodomain comprising at least one extracellular ligand-binding domain and a hinge;
b) at least one transmembrane domain; and
c) at least one endodomain comprising at least one death domain derived from Fas (CD95).

69. The cell death inducing CAR according to claim 68, wherein the death domain comprises an amino acid sequence having at least 80% sequence identity with SEQ ID NO: 6.

70. The cell death inducing CAR according to claim 68, wherein the at least one death domain derived from Fas (CD95) comprises one or more amino acid substitutions compared to the amino acid sequence of a wild type death domain from Fas (CD95), wherein the amino acid substitution(s) attenuate(s) self-association and/or binding to a pro-apoptotic or pro-necrotic adaptor protein.

71. The cell death inducing CAR according to claim 70, wherein the one or more amino acid substitutions are at positions corresponding to positions in the full length amino acid sequence of Fas (SEQ ID NO: 1), and wherein the one or more amino acid substitutions are selected from the group consisting of V245, R250, K251, V254, E256, K258, I259, D260, E261, K263, E272, W281, Y291, K296 and L298.

72. The cell death inducing CAR according to claim 68, wherein the at least one death domain comprises an amino acid sequence having at least 80% sequence identity with SEQ ID NO: 6, wherein the amino acid sequence comprises at least one amino acid substitution at a position which corresponds to a position in SEQ ID NO: 6, wherein the at least one amino acid substitution is selected from the group consisting of V16, R21, K22, V25, E27, K29, I30, D31, E32, K34, E43, W52, Y62, K67 and L69.

73. The cell death inducing CAR according to claim 72, wherein the at least one amino acid substitution comprises K67A.

74. The cell death inducing CAR according to claim 68, wherein the at least one death domain comprises one or more amino acid substitutions at positions corresponding to SEQ ID NO: 6 selected from the group consisting of R21, V25, E27, D31, E32, K34, Y62 and K67.

75. The cell death inducing CAR according to claim 74, wherein the one or more amino acid substitutions comprise the K67A substitution.

76. The cell death inducing CAR according to claim 68, wherein the at least one death domain derived from Fas (CD95) is derived from a Fas (CD95) intracellular domain.

77. The cell death inducing CAR according to claim 68, wherein the hinge is derived from a Fas (CD95) extracellular domain.

78. The cell death inducing CAR according to claim 68, wherein the hinge comprises an amino acid sequence having at least 80% sequence identity with any one of SEQ ID NOs: 16 to 18.

79. The cell death inducing CAR according to claim 68, wherein the at least one transmembrane domain is selected from the group consisting of CD95 (Fas) transmembrane domain, DR4 transmembrane domain, DR5 transmembrane domain, TNFR1 transmembrane domain, DR3 transmembrane domain, CD8 alpha transmembrane domain, 4-1BB transmembrane domain, DAP10 transmembrane domain and CD28 transmembrane domain.

80. The cell death inducing CAR according to claim 79, wherein the at least one transmembrane domain is a CD95 (Fas) transmembrane domain.

81. The cell death inducing CAR according to claim 68, wherein the at least one transmembrane domain comprises one or more amino acid substitutions compared to the amino acid sequence of the wild type transmembrane domain from which it is derived, wherein the one or more amino acid substitution(s) attenuate(s) self-association of the cell death inducing chimeric antigen receptor.

82. The cell death inducing CAR according to claim 68, wherein the at least one transmembrane domain comprises an amino acid sequence having at least 80% sequence identity with SEQ ID NO: 25, wherein the amino acid sequence comprises at least one amino acid substitution at a position which corresponds to a position in SEQ ID NO: 25 selected from the group consisting of C5, L7, L7, P10, I11, P12, L13 and I14.

83. The cell death inducing CAR according to claim 82, wherein the at least one amino acid substitution is selected from the group consisting of C5R, C5A, L7F, L7A, P10L, P10A, I11A, P12A, L13A and I14A.

84. The cell death inducing CAR according to claim 68, wherein the at least one extracellular ligand-binding domain comprises an extracellular antigen-binding domain.

85. The cell death inducing CAR according to claim 68, wherein the at least one extracellular ligand-binding domain is specific for a cell surface antigen N, wherein N is expressed on a non-pathological or healthy cell but not expressed on a targeted pathological (e.g., cancerous) cell.

86. The cell death inducing CAR according to claim 85, wherein the cell surface antigen is selected from the group consisting of CD56, CD205, CD83, CD206, CD200, CD36, RARRES1, Troponin C, Beta-1 integrin, CCKBR, GALR1, CD4, CD20, CD22, CD25, CD34, MUC1 and EGFRVIII.

87. A polynucleotide comprising a nucleic acid sequence encoding the cell death inducing CAR according to claim 68.

88. An expression vector comprising the polynucleotide according to claim 87.

89. A stem cell or cell derived therefrom comprising at least one cell death inducing CAR according to claim 68.

90. An immune cell comprising at least one cell death inducing CAR according to claim 68.

91. The immune cell according to claim 90, further comprising an activating CAR.

92. The immune cell according to claim 91, wherein the activating CAR comprises:
a) at least one ectodomain which comprises an extracellular ligand-binding domain;
b) at least one transmembrane domain; and
c) at least one endodomain which comprises a signal transducing domain and optionally a co-stimulatory domain.

93. The immune cell according to claim 92, wherein the extracellular ligand-binding domain of the activating CAR is specific for a cell surface antigen P, wherein P is expressed or overexpressed on a targeted pathological cell.

94. The immune cell according to claim 93, wherein the extracellular ligand-binding domain of the activating CAR is specific for cell surface antigen selected from the group consisting of the activating CAR is specific for a target antigen selected from the group consisting of CD123, ROR1, BCMA, PSMA, CD33, CD38, CD22, CS1, CLL-1, HSP70, EGFRVIII, FLT3, WT1, CD30, CD70, MUC1, MUC16, MUC17, PRAME, TSPAN10, Claudin18.2, DLL3, LY6G6D and GD2 (including O-acetyl-GD2).

95. The immune cell according to claim 90, wherein said immune cell is a T cell.

96. The immune cell according to claim 90, wherein a gene(s) encoding beta 2-microglobulin (B2M) and/or a gene encoding class II major histocompatibility complex transactivator (CIITA) has/have been inactivated.

97. The immune cell according to claim 90, wherein at least one gene encoding a component of a T-cell receptor (TCR) has been inactivated.

98. The immune cell according to claim 90, wherein said immune cell has been modified to confer resistance to at least one immune suppressive drug, chemotherapy drug, or anti-cancer drug.

99. A population of immune cells according of claim 90.

100. A composition comprising the immune cell according to claim 90 or the population of immune cells according to claim 99 for use as a medicament.


SEQUENCE LISTING (126 sequences)

SEQ ID NO: 1 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 2 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 3 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 4 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 5 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 6 - protein, 85 aa, Homo sapiens
SKYITTIAGVMTLSQVKGFVRKNGVNEAKIDEIKNDNVQDTAEQKVQLLRNWHQLHGKKEAYDTLIKDLKKANLCTLAEKIQTII

SEQ ID NO: 7 - protein, 84 aa, Homo sapiens
MLFFDKFANIVPFDSWDQLMRQLDLTKNEIDVVRAGTAGPGDALYAMLMKWVNKTGRNASIHTLLDALERMEERHAREKIQDLL

SEQ ID NO: 8 - protein, 84 aa, Homo sapiens
RQCFDDFADLVPFDSWEPLMRKLGLMDNEIKVAKAEAAGHRDTLYTMLIKWVNKTGRDASVHTLLDALETLGERLAKQKIEDHL

SEQ ID NO: 9 - protein, 86 aa, Homo sapiens
PATLYAVVENVPPLRWKEFVRRLGLSDHEIDRLELQNGRCLREAQYSMLATWRRRTPRREATLELLGRVLRDMDLLGCLEDIEEAL

SEQ ID NO: 10 - protein, 82 aa, Homo sapiens
GPQLYDVMDAVPARRWKEFVRTLGLREAEIEAVEVEIGRFRDQQYEMLKRWRQQQPAGLGAVYAALERMGLDGCVEDLRSRL

SEQ ID NO: 11 - protein, 145 aa, Homo sapiens
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 12 - protein, 206 aa, Homo sapiens
CIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 13 - protein, 209 aa, Homo sapiens
CKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 14 - protein, 221 aa, Homo sapiens
RYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 15 - protein, 197 aa, Homo sapiens
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 16 - protein, 7 aa, Homo sapiens
EEGSRSN

SEQ ID NO: 17 - protein, 46 aa, Homo sapiens
RCKPNFFCNSTVCEHCDPCTKCEHGIIKECTLTSNTKCKEEGSRSN

SEQ ID NO: 18 - protein, 90 aa, Homo sapiens
PCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 19 - protein, 10 aa, Homo sapiens
VHKESGNGHN

SEQ ID NO: 20 - protein, 32 aa, Homo sapiens
VHKESGTKHSGEVPAVEETVTSSPGTPASPCS

SEQ ID NO: 21 - protein, 7 aa, Homo sapiens
AVCGWRQ

SEQ ID NO: 22 - protein, 45 aa, Homo sapiens
TTTPAPRPPTPAPTIASQPLSLRPEACRPAAGGAVHTRGLDFACD

SEQ ID NO: 23 - protein, 231 aa, Homo sapiens
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 16 aa, Homo sapiens
GLAVSTISSFFPPGYQ

SEQ ID NO: 25 - protein, 17 aa, Homo sapiens
LGWLCLLLLPIPLIVWV

SEQ ID NO: 26 - protein, 23 aa, Homo sapiens
IWVILVVTLVVPLLLVAVLIVCC

SEQ ID NO: 27 - protein, 21 aa, Homo sapiens
LSGIIIGVTVAAVVLIVAVFV

SEQ ID NO: 28 - protein, 23 aa, Homo sapiens
VLLPLVIFFGLCLLSLLFIGLMY

SEQ ID NO: 29 - protein, 21 aa, Homo sapiens
MFWVQVLLAGLVVPLLLGATL

SEQ ID NO: 30 - protein, 24 aa, Homo sapiens
IYIWAPLAGTCGVLLLSLVITLYC

SEQ ID NO: 31 - protein, 27 aa, Homo sapiens
IISFFLALTSTALLFLLFFLTLRFSVV

SEQ ID NO: 32 - protein, 21 aa, Homo sapiens
LLAGLVAADAVASLLIVGAVF

SEQ ID NO: 33 - protein, 27 aa, Homo sapiens
FWVLVVVGGVLACYSLLVTVAFIIFWV

SEQ ID NO: 34 - protein, 52 aa, Homo sapiens
FFIPLLVVILFAVDTGLFISTQQQVTFLLKIKRTRKGFRLLNPHPKPNPKNN

SEQ ID NO: 35 - protein, 215 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 36 - protein, 43 aa, Homo sapiens
LGEPQLCYILDAILFLYGIVLTLLYCRLKIQVRKAAITSYEKS

SEQ ID NO: 37 - protein, 15 aa, Artificial Sequence
  peptide linker
GGGGSGGGGSGGGGS

SEQ ID NO: 38 - protein, 9 aa, Artificial Sequence
  mimotope
CPYSNPSLC

SEQ ID NO: 39 - protein, 12 aa, Artificial Sequence
  Mimotope
CQFDLSTRRLKC

SEQ ID NO: 40 - protein, 12 aa, Artificial Sequence
  Mimotope
CQYNLSSRALKC

SEQ ID NO: 41 - protein, 12 aa, Artificial Sequence
  mimotope
CVWQRWQKSYVC

SEQ ID NO: 42 - protein, 12 aa, Artificial Sequence
  Mimotope
CMWDRFSRWYKC

SEQ ID NO: 43 - protein, 24 aa, Artificial Sequence
  Mimotope
NSELLSLINDMPITNDQKKLMSNN

SEQ ID NO: 44 - protein, 25 aa, Artificial Sequence
  Mimotope
SFVLNWYRMSPSNQTDKLAAFPEDR

SEQ ID NO: 45 - protein, 19 aa, Artificial Sequence
  Mimotope
SGTYLCGAISLAPKAQIKE

SEQ ID NO: 46 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 215 aa, Homo sapiens; identical to SEQ ID NO: 35

SEQ ID NO: 48 - protein, 43 aa, Homo sapiens; identical to SEQ ID NO: 36

SEQ ID NO: 49 - protein, 42 aa, Homo sapiens
KRGRKKLLYIFKQPFMRPVQTTQEEDGCSCRFPEEEEGGCEL

SEQ ID NO: 50 - DNA, 544 bp, Homo sapiens
taaaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccg

SEQ ID NO: 51 - DNA, 141 bp, Homo sapiens
gtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccg

SEQ ID NO: 52 - DNA, 244 bp, Homo sapiens
gcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccg

SEQ ID NO: 53 - DNA, 347 bp, Homo sapiens
gcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccg

SEQ ID NO: 54 - DNA, 445 bp, Homo sapiens
gccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccg

SEQ ID NO: 55 - DNA, 245 bp, Homo sapiens
taaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 56 - DNA, 1212 bp, Homo sapiens
cctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacaagggcgaattccagcacactgg

SEQ ID NO: 57 - DNA, 938 bp, Homo sapiens
taagtttgctctgtcttccacctgttgcaggagatagtgccaagccctccacctctgccaggggctgtggccttgcctctacctgaattcgaactgttgccctactctccaaccatgattaatgggtgttgtcctggcctctgactacagcaggggccgttactatgccctcttgaagacatgaggttgtcctgtctgcctcctgaaacaggctgttttccagcattctgtctgtaagagggatggtagcctgccattcacctacccttgactataataaagctactgttccatgccctgagatgacatgggaattgttctctcggcctgacctgactgtaacatgcatggtctgctcaccagctatttaacagggatattgtcctctcctctgactctgaacgatgctacctttgctgccagacagaaacaaaagggtctctcagctgcaacggtggtgctgaggtgctgtttgcctctcaccataagctgagtgtgtgtccgcttgccccctgctcactgggcccaaaggctgcccttgaatctcttgcccagatgcaccctggagggcagaagggagggtctatcagacatcctcccctcaactttaaacctcccagtgtcaccctgggacagtaggggaagatggacctggtctggagatgtaggggacccccaggggctgagaggcaggggtctatggtggcaggaagcttggcgtgctagagggttgtggttgggctgctggggcccggttggctgcggagccctccggaggaggcaggaagtcagggtgggacgtgggcgcggggagacaggtggtggctacgacggcgaagggagctgagactgtccaggcagccaggttaggccaggaggaccatgtgaatggggccagagggctcccgggctgggcaggtaaggagcgctggtattgg

SEQ ID NO: 58 - DNA, 923 bp, Homo sapiens
taagttccatagatgtccccagtgtcctagtccgtgcatcagctcgcgcactcggagggactctaggcagggggagggccccgcggccagtatgtgcgtccgaggctttcccgcagggggcagtgccgcccgcccgcgcgccgatacggtgggagggggtgggaacctgcgcggagttctggaggttctttgggagaaagttaggggatgcggaggggtgggcgcaagacttccaggactccagggaggccgtggggagggccgccgagggtgcagtgtgaggcgcaggagggggttgggggcggtgcacgttgcagggagacgcagcccctggaagatgcgagtgtgaacgtgtgagtgtgagtgcgtgtgtatgtgtgtgtgtgcgcgcgcaccgcagctctccgggttccgcgaggcgcgcgggtgtcagcttgcagccggggctcctccctccggcccccctgcccagcccggcggtccctcctccctccctccccgctcgcccctccccggcgggccaggggctgggacgccccggcggagcaggcggcggcggtggcgagttggggagccctaggctcggcgctgccggaggggcccgagccgagccgcctgcgccccggccgggcagcgccgggcccgcttcccgcggggccacgccctgtcaaactttgttgcggcggctagcgcagcgggcccgcaagcgggcgggaggggcgccgggccgggccgggcagggcgcgggcggctaggggctccgagagcggcggccccggcccgcggccccaccatgccccagctcggcggcgggggcggcggcggcggcggcggcagcgggggaggcggcggctccagcgccggggcggccggcggaggggacgacctcggggcgaacgacgagctgatccccttccaggacg

SEQ ID NO: 59 - DNA, 1046 bp, Homo sapiens
taattttggcatatccatcctagtgggacttaacatttcatgaaatgattcactactaataaacaaaaaagggaggagggaggcctcatgggttaatagtttctttcattgtagatgaccaggaactttgaccagccccttcacttccccaagctctctgaaagtggagggttgatctttccttttgaccacttttgtcgcatcccgtctaagggtggctgatttcactgctgaattaaccaccaagcacccccccaccccctcccccagccaccactttctcaaatactacccttccttttccccctcccttaagacttatttctaatatttctaaagtgcactgttttgggcctctccccatccccgccccccaagtgggctttccttccgccttcctcggctcggattcctgacttggtcgccacccccttctcctcctctcccaccccgcattgtctttctgaaaccgccccctcccggagcaagtccctgcaccctcgcccagaatcccgggctcgcacacactccgcgcaggccgctccccctgcacactcctccctccgtctccccccggcttccccgcccctctcttcctccttctttccctcctccctctcccggcgcccgaaaggatcattgttagccgcccccgccccgcccaccccggctgtttatttatgcacacgtcactgggccggccccgccctccggcatctcattaaggcagtgtgttcctctcgccctgtcaataatctccgctcccagactactccgttcctccggatttcgatccccctttttctatctgtcaatcagcgccgcctttgaactgaaaagctctcagtctaacttcaactcactcaaatccgagcggcacgagcacctcctgtatcttcggcttccccccccctttgctctttatatctgacttcttgttgttgttggtgttttttttttttttaccccccttttttatttattatttttttgcacattgatcggatccttgggaacgagagaaaaaagaaacccaaactcacgcg

SEQ ID NO: 60 - DNA, 910 bp, Homo sapiens
taaaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggtgagcccccgccggcgccggctcctcccccgcggtcgccgcgccgcgccgccccagttgcgcgcggccctcggggtctccagcgcgcagagcgtccctgccccggcgtcggccccgacccccgcggtcccaccgcccctcactcccctccggttctccctccaggctctcgggcgggaacacgctgcgcagagactcttcccggacaaacttccagagcccctggaggacggtgagtttctgcccggcccggcttcccttcgtcgcgctcaggccctggcctcggtgggacggggacgccaaggaccgcggggagccgggtgcctcccccaccgcagctcaggaggcggcagaacccaggggtggagagtggggggcgggcttcccgggcgccgccgggtcgagtcacttccggtgccctgacctttataggagtaaacagacccccgccatccccgcctcccctcctgcccaggtgactgactaatccgccgccttcaggagacagaattggccaaggtttcttggttggagggcggggggtgggaggtcaagtaggggccacctcggggaggcctgccctccaggtccttcccctaaaacttggcactgccgatactcccagcccgttccttcccaagtcaggaacttgcaggggaccccttggcaattctttttctctcaagagcagacagccttcagtcccagcgctgccagggctggtgtgtctgacccagctgtggtttttg

SEQ ID NO: 61 - protein, 21 aa, Artificial Sequence
  signal peptide
MALPVTALLLPLALLLHAARP

SEQ ID NO: 62 - protein, 20 aa, Artificial Sequence
  signal peptide
METDTLLLWVLLLWVPGSTG

SEQ ID NO: 63 - DNA, 63 bp, Artificial Sequence
  CD8 alpha signal sequence
atggctttgcctgtcactgccttgctgcttccacttgctctgttgttgcacgccgcaagaccc

SEQ ID NO: 64 - DNA, 744 bp, Artificial Sequence
  CD20 targeting scFv
gaagtccagctggtcgaatctgggggcgggctggtgcagccaggaagatcactgaggctgagctgcgccgcttccggcttcaccttcaacgactatgccatgcactgggtgagacaggctcccggaaagggcctggagtgggtctctaccatcagttggaattccgggtctattggatatgccgacagcgtgaaaggccgcttcacaatctctcgagataacgctaagaaaagtctgtacctgcagatgaattcactgagggcagaggacactgccctgtactattgcgccaaggatattcagtacggcaactactattacgggatggacgtctgggggcagggaaccacagtgaccgtcagctccggaggaggaggatccggaggaggaggaagcggaggaggaggatccgagatcgtgctgacacagagcccagccactctgagtctgtcacccggcgaacgagctacactgtcctgtcgggcaagccagtccgtctctagttatctggcttggtaccagcagaagccaggacaggcaccacgactgctgatctacgatgctagcaacagagcaacagggattcctgcaaggttctctggcagtgggtcaggaactgactttacactgactatctcaagcctggagcctgaagatttcgccgtgtattactgccagcagcggtccaattggccaatcacctttggccaggggacacgcctggagatcaagcggtcagatccg

SEQ ID NO: 65 - DNA, 738 bp, Artificial Sequence
  FMC63 targeting scFv
gaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatccc

SEQ ID NO: 66 - DNA, 135 bp, Homo sapiens
accacaacccccgctccaaggccccctacccccgcaccaactattgcctcccagccactctcactgcggcctgaggcctgtcggcccgctgctggaggcgcagtgcatacaaggggcctcgatttcgcctgcgat

SEQ ID NO: 67 - DNA, 699 bp, Homo sapiens
gagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggat

SEQ ID NO: 68 - DNA, 51 bp, Homo sapiens
ttggggtggctttgccttcttcttttgccaattccactaattgtttgggtg

SEQ ID NO: 69 - DNA, 435 bp, Homo sapiens
aagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtc

SEQ ID NO: 70 - DNA, 69 bp, Artificial Sequence
  2A cis-acting hydrolase element
ggaggaagcggagagggacgaggaagcctgctgacctgcggggacgtggaggaaaacccaggacctcat

SEQ ID NO: 71 - DNA, 726 bp, Artificial Sequence
  EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcggccgac

SEQ ID NO: 72 - DNA, 699 bp, Artificial Sequence
  BFP
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 73 - DNA, 492 bp, Artificial Sequence
  SFFV pomoter
gataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggg

SEQ ID NO: 74 - DNA, 938 bp, Artificial Sequence; identical to SEQ ID NO: 57
  Lck promoter

SEQ ID NO: 75 - DNA, 1188 bp, Artificial Sequence
  EF1a promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 76 - DNA, 1428 bp, Artificial Sequence
  FMC63-CD8a-CD95-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccaccacaacccccgctccaaggccccctacccccgcaccaactattgcctcccagccactctcactgcggcctgaggcctgtcggcccgctgctggaggcgcagtgcatacaaggggcctcgatttcgcctgcgatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaa

SEQ ID NO: 77 - DNA, 1434 bp, Artificial Sequence
  CD20_C203H-CD8a-CD95-2A-BFP
atggctctgcctgtcaccgctctgctgctgcccctggctctgctgctgcacgccgcacgccccgaagtccagctggtcgaatctgggggcgggctggtgcagccaggaagatcactgaggctgagctgcgccgcttccggcttcaccttcaacgactatgccatgcactgggtgagacaggctcccggaaagggcctggagtgggtctctaccatcagttggaattccgggtctattggatatgccgacagcgtgaaaggccgcttcacaatctctcgagataacgctaagaaaagtctgtacctgcagatgaattcactgagggcagaggacactgccctgtactattgcgccaaggatattcagtacggcaactactattacgggatggacgtctgggggcagggaaccacagtgaccgtcagctccggaggaggaggatccggaggaggaggaagcggaggaggaggatccgagatcgtgctgacacagagcccagccactctgagtctgtcacccggcgaacgagctacactgtcctgtcgggcaagccagtccgtctctagttatctggcttggtaccagcagaagccaggacaggcaccacgactgctgatctacgatgctagcaacagagcaacagggattcctgcaaggttctctggcagtgggtcaggaactgactttacactgactatctcaagcctggagcctgaagatttcgccgtgtattactgccagcagcggtccaattggccaatcacctttggccaggggacacgcctggagatcaagcggtcggatcccaccacaacccccgctccaaggccccctacccccgcaccaactattgcctcccagccactctcactgcggcctgaggcctgtcggcccgctgctggaggcgcagtgcatacaaggggcctcgatttcgcctgcgatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaa

SEQ ID NO: 78 - DNA, 1992 bp, Artificial Sequence
  FMC63-IgG1-CD95-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaa

SEQ ID NO: 79 - DNA, 1998 bp, Artificial Sequence
  CD20_C203H-IgG1-CD95-2A-BFP
atggctctgcctgtcaccgctctgctgctgcccctggctctgctgctgcacgccgcacgccccgaagtccagctggtcgaatctgggggcgggctggtgcagccaggaagatcactgaggctgagctgcgccgcttccggcttcaccttcaacgactatgccatgcactgggtgagacaggctcccggaaagggcctggagtgggtctctaccatcagttggaattccgggtctattggatatgccgacagcgtgaaaggccgcttcacaatctctcgagataacgctaagaaaagtctgtacctgcagatgaattcactgagggcagaggacactgccctgtactattgcgccaaggatattcagtacggcaactactattacgggatggacgtctgggggcagggaaccacagtgaccgtcagctccggaggaggaggatccggaggaggaggaagcggaggaggaggatccgagatcgtgctgacacagagcccagccactctgagtctgtcacccggcgaacgagctacactgtcctgtcgggcaagccagtccgtctctagttatctggcttggtaccagcagaagccaggacaggcaccacgactgctgatctacgatgctagcaacagagcaacagggattcctgcaaggttctctggcagtgggtcaggaactgactttacactgactatctcaagcctggagcctgaagatttcgccgtgtattactgccagcagcggtccaattggccaatcacctttggccaggggacacgcctggagatcaagcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaa

SEQ ID NO: 80 - DNA, 2769 bp, Artificial Sequence
  FMC63-IgG1-CD95_R250E-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttgaaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 81 - DNA, 2769 bp, Artificial Sequence
  FMC63-IgG1-CD95_Y291E-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcggaagacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 82 - DNA, 2769 bp, Artificial Sequence
  FMC63-IgG1-CD95_K296A-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattgcagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 83 - DNA, 2769 bp, Artificial Sequence
  FMC63-CD8-CD95_R250A-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttgcaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 84 - DNA, 2769 bp, Artificial Sequence
  FMC63-CD8-CD95_V254N-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtaataatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 85 - DNA, 2769 bp, Artificial Sequence
  FMC63-CD8-CD95_E256A-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgctgccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 86 - DNA, 2769 bp, Artificial Sequence
  FMC63-CD8-CD95_D260A-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagctgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 87 - DNA, 2769 bp, Artificial Sequence
  FMC63-CD8-CD95_E261A-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgctatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 88 - DNA, 2769 bp, Artificial Sequence
  pCLS30072 (FMC63-CD8-CD95_K263A-2A-BFP)
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcgctaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 89 - DNA, 2769 bp, Artificial Sequence
  FMC63-CD8-CD95_Y291D-2A-BFP
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcggacgacacattgattaaagatctcaaaaaagccaatctttgtactcttgcagagaaaattcagactatcatcctcaaggacattactagtgactcagaaaattcaaacttcagaaatgaaatccagagcttggtcgaatcccatggaggaagcggagagggacgaggaagcctgctgacctgcggggacgtggaggaaaacccaggacctcatatgatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 90 - DNA, 795 bp, Artificial Sequence
  PSMA targeting scFV
gggagcggcagtggggcccaggcggccgaagtgcagctgcaacagtccggacctgaactcgtgaagcccggaacctccgtcagaatcagctgcaagaccagcggatacaccttcaccgagtacaccatccactgggtgaagcagagtcacgggaagtccctggagtggatcgggaacatcaacccaaacaacggggggaccacctacaaccagaagttcgaggacaaggctaccctgaccgtggacaagagctcctccaccgcctacatggagctgaggtccctcacctccgaggactccgctgtctactactgcgccgctgggtggaacttcgactactggggacagggaaccaccgtgacagtgtcaagtggagggggaggatctggaggcggcgggagtgggggaggaggcagtgatattgtgatgactcagtcccacaagttcatgagcactagcgtgggcgacagagtctccatcatctgcaaggccagccaggacgtgggcacagctgtggactggtaccagcagaagccagggcagtcccctaagctgctcatctactgggccagcacaaggcacacaggcgtgccagacaggttcacaggcagcgggtcaggcacagacttcactctgactattactaacgtccagagcgaggatctggccgattacttctgccagcagtacaatagctaccccctgacatttggcgccgggacaatgctggatctgaaacgcgccgcagctcggtcagatccggcctcaggggccggatccggcagcggaagcgct

SEQ ID NO: 91 - DNA, 30 bp, Homo sapiens
gtccacaaagaatcaggcaatggacataat

SEQ ID NO: 92 - DNA, 96 bp, Homo sapiens
gtccacaaagaatcaggtacaaagcacagtggggaagccccagctgtggaggagacggtgacctccagcccagggactcctgcctctccctgttct

SEQ ID NO: 93 - DNA, 69 bp, Homo sapiens
atatgggtgattttggttgtgactttggttgttccgttgctgttggtggctgtgctgattgtctgttgt

SEQ ID NO: 94 - DNA, 63 bp, Homo sapiens
ctctcaggcatcatcataggagtcacagttgcagccgtagtcttgattgtggctgtgtttgtt

SEQ ID NO: 95 - DNA, 618 bp, Homo sapiens
tgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggag

SEQ ID NO: 96 - DNA, 627 bp, Homo sapiens
tgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcc

SEQ ID NO: 97 - DNA, 2376 bp, Artificial Sequence
  PSMA-DR4-EGFP
atggctttgcctgtcactgccttgctgcttccacttgctctgttgttgcacgccgcaagacccgggagcggcagtggggcccaggcggccgaagtgcagctgcaacagtccggacctgaactcgtgaagcccggaacctccgtcagaatcagctgcaagaccagcggatacaccttcaccgagtacaccatccactgggtgaagcagagtcacgggaagtccctggagtggatcgggaacatcaacccaaacaacggggggaccacctacaaccagaagttcgaggacaaggctaccctgaccgtggacaagagctcctccaccgcctacatggagctgaggtccctcacctccgaggactccgctgtctactactgcgccgctgggtggaacttcgactactggggacagggaaccaccgtgacagtgtcaagtggagggggaggatctggaggcggcgggagtgggggaggaggcagtgatattgtgatgactcagtcccacaagttcatgagcactagcgtgggcgacagagtctccatcatctgcaaggccagccaggacgtgggcacagctgtggactggtaccagcagaagccagggcagtcccctaagctgctcatctactgggccagcacaaggcacacaggcgtgccagacaggttcacaggcagcgggtcaggcacagacttcactctgactattactaacgtccagagcgaggatctggccgattacttctgccagcagtacaatagctaccccctgacatttggcgccgggacaatgctggatctgaaacgcgccgcagctcggtcagatccggcctcaggggccggatccggcagcggaagcgctgtccacaaagaatcaggcaatggacataatatatgggtgattttggttgtgactttggttgttccgttgctgttggtggctgtgctgattgtctgttgttgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggagagccatggaggaagcggagagggacgaggaagcctgctgacctgcggggacgtggaggaaaacccaggacctcatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcggccgac

SEQ ID NO: 98 - DNA, 2445 bp, Artificial Sequence
  PSMA-DR5-EGFP
atggctttgcctgtcactgccttgctgcttccacttgctctgttgttgcacgccgcaagacccgggagcggcagtggggcccaggcggccgaagtgcagctgcaacagtccggacctgaactcgtgaagcccggaacctccgtcagaatcagctgcaagaccagcggatacaccttcaccgagtacaccatccactgggtgaagcagagtcacgggaagtccctggagtggatcgggaacatcaacccaaacaacggggggaccacctacaaccagaagttcgaggacaaggctaccctgaccgtggacaagagctcctccaccgcctacatggagctgaggtccctcacctccgaggactccgctgtctactactgcgccgctgggtggaacttcgactactggggacagggaaccaccgtgacagtgtcaagtggagggggaggatctggaggcggcgggagtgggggaggaggcagtgatattgtgatgactcagtcccacaagttcatgagcactagcgtgggcgacagagtctccatcatctgcaaggccagccaggacgtgggcacagctgtggactggtaccagcagaagccagggcagtcccctaagctgctcatctactgggccagcacaaggcacacaggcgtgccagacaggttcacaggcagcgggtcaggcacagacttcactctgactattactaacgtccagagcgaggatctggccgattacttctgccagcagtacaatagctaccccctgacatttggcgccgggacaatgctggatctgaaacgcgccgcagctcggtcagatccggcctcaggggccggatccggcagcggaagcgctgtccacaaagaatcaggtacaaagcacagtggggaagccccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtccagccatggaggaagcggagagggacgaggaagcctgctgacctgcggggacgtggaggaaaacccaggacctcatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcggccgac

SEQ ID NO: 99 - DNA, 34 bp, Artificial Sequence
  oligonucleotide
gtatgacacattgattgcagatctcaaaaaagcc

SEQ ID NO: 100 - DNA, 999 bp, Homo sapiens
cctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccag

SEQ ID NO: 101 - DNA, 1000 bp, Homo sapiens
ccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaat

SEQ ID NO: 102 - DNA, 2814 bp, Artificial Sequence
  TRAC-L
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 103 - DNA, 2832 bp, Artificial Sequence
  TRAC-R
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 104 - DNA, 49 bp, Homo sapiens
ttgtcccacagatatccagaaccctgaccctgccgtgtaccagctgaga

SEQ ID NO: 105 - DNA, 60 bp, Artificial Sequence
  2A-element
tccggtgagggcagaggaagtcttctaacatgcggtgacgtggaggagaatccgggcccc

SEQ ID NO: 106 - DNA, 1989 bp, Artificial Sequence
  D-CAR2 coding sequence without start codon
gctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccagagcttggtcgaa

SEQ ID NO: 107 - DNA, 1989 bp, Artificial Sequence
  D-CAR3 coding sequence without start codon
gctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattgcagatctcaaaaaagccaatctttgtactcttgcagagaaaattcagactatcatcctcaaggacattactagtgactcagaaaattcaaacttcagaaatgaaatccagagcttggtcgaa

SEQ ID NO: 108 - DNA, 276 bp, Bos taurus
tctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattc

SEQ ID NO: 109 - DNA, 11 bp, Artificial Sequence
  5′ UTR
tcgacgccacc

SEQ ID NO: 110 - DNA, 1992 bp, Artificial Sequence; identical to SEQ ID NO: 78
  D-CAR2 coding sequence with start codon

SEQ ID NO: 111 - DNA, 1992 bp, Artificial Sequence
  D-CAR3 coding sequence with start codon
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattgcagatctcaaaaaagccaatctttgtactcttgcagagaaaattcagactatcatcctcaaggacattactagtgactcagaaaattcaaacttcagaaatgaaatccagagcttggtcgaa

SEQ ID NO: 112 - DNA, 1000 bp, artificial sequence
  PD1 left homology
ccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacc

SEQ ID NO: 113 - DNA, 1000 bp, artificial sequence
  PD1 right homology
gcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcc

SEQ ID NO: 114 - DNA, 2781 bp, artificial sequence
  PD1_T3C-L2
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtggtggctatcgcttccaagctggggggaaagcaggccctggagaccgtccaggcccttctcccagtgctttgccaggctcacggactgacccctgaacaggtggtggcaattgcctcacacgacgggggcaagcaggcactggagactgtccagcggctgctgcctgtcctctgccaggcccacggactcactcctgagcaggtcgtggccattgccagccacgatgggggcaaacaggctctggagaccgtgcagcgcctcctcccagtgctgtgccaggctcatgggctgaccccacagcaggtcgtcgccattgccagtaacggcggggggaagcaggccctcgaaacagtgcagaggctgctgcccgtcttgtgccaagcacacggcctgacacccgagcaggtggtggccatcgcctctcatgacggcggcaagcaggcccttgagacagtgcagagactgttgcccgtgttgtgtcaggcccacgggttgacaccccagcaggtggtcgccatcgccagcaatggcgggggaaagcaggcccttgagaccgtgcagcggttgcttccagtgttgtgccaggcacacggactgacccctcaacaggtggtcgcaatcgccagctacaagggcggaaagcaggctctggagacagtgcagcgcctcctgcccgtgctgtgtcaggctcacggactgacaccacagcaggtggtcgccatcgccagtaacgggggcggcaagcaggctttggagaccgtccagagactcctccccgtcctttgccaggcccacgggttgacacctcagcaggtcgtcgccattgcctccaacaacgggggcaagcaggccctcgaaactgtgcagaggctgctgcctgtgctgtgccaggctcatgggctgacaccccagcaggtggtggccattgcctctaacaacggcggcaaacaggcactggagaccgtgcaaaggctgctgcccgtcctctgccaagcccacgggctcactccacagcaggtcgtggccatcgcctcaaacaatggcgggaagcaggccctggagactgtgcaaaggctgctccctgtgctctgccaggcacacggactgacccctcagcaggtggtggcaatcgcttccaacaacgggggaaagcaggccctcgaaaccgtgcagcgcctcctcccagtgctgtgccaggcacatggcctcacacccgagcaagtggtggctatcgccagccacgacggagggaagcaggctctggagaccgtgcagaggctgctgcctgtcctgtgccaggcccacgggcttactccagagcaggtcgtcgccatcgccagtcatgatggggggaagcaggcccttgagacagtccagcggctgctgccagtcctttgccaggctcacggcttgactcccgagcaggtcgtggccattgcctcaaacattgggggcaaacaggccctggagacagtgcaggccctgctgcccgtgttgtgtcaggcccacggcttgacaccccagcaggtggtcgccattgcctctaatggcggcgggagacccgccttggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 115 - DNA, 2778 bp, artificial sequence
  PD1T3R
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 116 - DNA, 49 bp, artificial sequence
  PD1-T3
tacctctgtggggccatctccctggcccccaaggcgcagatcaaagaga

SEQ ID NO: 117 - DNA, 60 bp, artificial sequence; identical to SEQ ID NO: 105
  2A-element

SEQ ID NO: 118 - DNA, 1989 bp, artificial sequence; identical to SEQ ID NO: 107
  apoptosis CAR

SEQ ID NO: 119 - DNA, 276 bp, artificial sequence; identical to SEQ ID NO: 108
  BGH polyA

SEQ ID NO: 120 - DNA, 1000 bp, artificial sequence
  Lck left homology
gggatagggggtgcctctgtgtgtgtgtgtgagagtgtgtgtgtgtagggtgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgtgtgtgaatttacttgtagcctgagggctcagagggagcaccggtttggagctgggaccccctattttagcttttctgtggctggtgaatggggatcccaggatctcacaatctcaggtacttttggaactttccagggcaaggccccattatatctgatgttgggggagcagatcttgggggagccccttcagccccctcttccattccctcagggacc

SEQ ID NO: 121 - protein, 219 aa, artificial sequence
  Interleukin-12 subunit alpha
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 122 - protein, 328 aa, artificial sequence
  Interleukin-12 subunit beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 123 - DNA, 1000 bp, artificial sequence
  lck right homology
ggctgtggctgcagctcacacccggaagatgactggatggaaaacatcgatgtgtgtgagaactgccattatcccatagtcccactggatggcaagggcacggtaagaggcgagacaggggccttggtgagggagttgggtagagaatgcaacccaggagaaagaaatgaccagcactacaggcccttgaaagaatagagtggccctctcccctgaaatacagaaaggaaaagaggcccagagaggggaagggaatctcctaagatcacacagaaagtagttggtaaactcagggataacatctaaccaggctggagaggctgagagcagagcaggggggaagggggccagggtctgacccaatcttctgctttctgaccccaccctcatcccccactccacagctgctcatccgaaatggctctgaggtgcgggacccactggttacctacgaaggctccaatccgccggcttccccactgcaaggtgaccccaggcagcagggcctgaaagacaaggcctgcggatccctggctgttggcttccacctctcccccacctactttctccccggtcttgccttccttgtcccccaccctgtaactccaggcttcctgccgatcccagctcggttctccctgatgccccttgtctttacagacaacctggttatcgctctgcacagctatgagccctctcacgacggagatctgggctttgagaagggggaacagctccgcatcctggagcagtgagtccctctccaccttgctctggcggagtccgtgagggagcggcgatctccgcgacccgcagccctcctgcggcccttgaccagctcggggtggccgcccttgggacaaaattcgaggctcagtattgctgagccagggttgggggaggctggcttaaggggtggaggggtctttgagggagggtctcaggtcgacggctgagcgagccacactgacccacctccgtggcgcaggagcggcgagtg

SEQ ID NO: 124 - DNA, 1992 bp, artificial sequence; identical to SEQ ID NO: 111
  apoptosis CAR

SEQ ID NO: 125 - DNA, 1000 bp, artificial sequence
  Lck left homology
ctcataacaattctatgaggtaggaacagttatttactctattttccaaataaggaaactgggctcgcccaaggttccacaactaacatgtgtgtattattgagcatttaatttacaccagggaagcaggttgtggtggtgtgcacctgttgtccagctatttaggaggctgaggtgaaaggatcacttgaacggaggagttcaaatttgcaatgtgctatgattgtgcctgtgaacagctgctgcactccagcctgggcaacatagtgagatcccttatctaaaacattttttttaagtaaataatcaggtgggcacggtggctcacgcctgtaatccagcactttgggaggctgaggcgggcggatcacctgaggtcaggagttcaagaccagcctgaccaacatggagaaacccgtctctactaaaaatacaaaattagcttggcgtggtggtgcatgcctgtaatcccagctactcgagaagctgaggcaggagaattgtttgaacctgggaggtggaggttgcggtgagccgagatcgcaccattgcactccagcctgggcaacaagagtgaaattgcatctcaaaaaaaaagaaaaggaaataatctataccaggcactccaagtggtgtgactgatattcaacaagtacctctagtgtgaccttaccattgatgaagaccaagattcttttggattggtgctcacactgtgccagttaaatattccgaacattacccttgcctgtgggcttccagtgcctgaccttgatgtcctttcacccatcaacccgtagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagcct

SEQ ID NO: 126 - DNA, 1000 bp, artificial sequence
  lck right homology
gaggccttgagaggccctggggttctccccctttctctccagcctgacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtacatgtgtagcctgtgcatgtatgtcttggacactgtacaaggtacccctttctggctctcccatttcctgagaccacagagagaggggagaagcctgggattgacagaagcttctgcccacctacttttctttcctcagatcatccagaagttcctcaagggccaggactttatctaatacctctgtgtgctcctccttggtgcctggcctggcacacatcaggagttcaataaatgtctgttgatgactgttgtacatctctttgctgtccactctttgtgggtgggcagtgggggttaagaaaatggtaattaggtcaccctgagttggggtgaaagatgggatgagtggatgtctggaggctctgcagaccccttcaaatgggacagtgctcctcacccctccccaaaggattcagggtgactcctacctggaatcccttagggaatgggtgcgtcaaaggaccttcctccccattataaaagggcaacagcattttttactgattcaagggctatatttgacctcagattttgtttttttaaggctagtcaaatgaagcggcgggaatggaggaggaacaaataaatctgtaactatcctcagatttttttttttttttgagactgggtctcactttttcatccaggctggagtgcagtcgcatgatcacggctcactgtagcctcaacctctccagctcaaatgctcctcctgtctcagcctcccgagtacctgggactactttcttgaggccaggaattcaagaacagagtaagatcctggtctccaaaaaaagttttaaa